(19) United States
(12) Patent Application Publication
(10) Pub. No.: US 2012/0077814 A1
(43) Pub. Date: Mar. 29, 2012

(54) SULFONAMIDE, SULFAMATE, AND SULFAMOTHIOATE DERIVATIVES

(76) Inventors: Zhong Wang, Cupertino, CA (US); Emilie D. Smith, Apex, NC (US); James M. Veal, Apex, NC (US); Kenneth H. Huang, Chapel Hill, NC (US); Robert N. Atkinson, Raleigh, NC (US); Rong Jiang, Fuquay Varina, NC (US)

(21) Appl. No.: 13/230,443

(22) Filed: Sep. 12, 2011

Related U.S. Application Data
(60) Provisional application No. 61/381,660, filed on Sep. 10, 2010.

(51) Int. Cl. A61K 31/53; A61K 31/519; A61P 25/28; A61P 35/00; A61P 29/00; C07D 487/04; C07D 495/04

(52) U.S. Cl. 514/243; 544/183; 544/262; 514/262.1; 544/278; 514/260.1; 544/212; 514/245

(57) ABSTRACT

The disclosure provides biologically active compounds of formula (I):

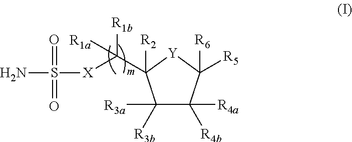

and pharmaceutically acceptable salts thereof, compositions containing these compounds, and methods of using these compounds in a variety applications, such as treatment of diseases or disorders associated with E1 type activating enzymes, and with Nedd8 activating enzyme (NAE) in particular.

### CROSS-REFERENCE TO RELATED APPLICATIONS

This application claims benefit of U.S. Provisional Application Ser. No. 61/381,660, filed Sep. 10, 2010, which is incorporated by reference herein in its entirety.

### BACKGROUND OF THE INVENTION

1. Field of the Invention

This disclosure provides compounds, compositions and methods for the treatment of various disorders. In particular, the disclosure provides sulfonamide, sulfamate, and sulfamothioate derivatives which inhibit the activity of E1 type activating enzymes, such as Nedd8 activating enzyme (NAE).

2. Description of Related Art

An important type of post-translational modification is the covalent attachment of ubiquitin (Ub) or ubiquitin-like proteins (Ubl's), such as Nedd8 and SUMO, to a particular protein thereby affecting its activities. Ubiquitin and ubiquitin-like proteins alter the molecular surface of target proteins and impact properties such as protein-protein interactions, enzymatic activity, stability and cellular localization of the target. As a result, they are involved in cellular processes such as the cell cycle, differentiation, stress response, inflammation, immune response, and apoptosis. These processes are critical to the proper functioning of a cell and, when they are defective, give rise to a range of disorders including cancer, inflammatory diseases, neurodegenerative diseases, and infections.

The process by which Ub and Ubl's are attached to a target protein is complex. Three families of protein ligases, generally termed E1, E2, or E3 ligases or enzymes, are involved and act in cascade fashion to accomplish the covalent modification. Ubiquitin, Nedd8, and SUMO as well as other Ubl's each have their own dedicated E1 ligase which initiates the transfer process. More specifically, the E1 enzyme catalyzes, via hydrolysis of ATP and release of pyrophosphate, the formation of an acyladenylate intermediate with the C-terminal glycine of the ubiquitin or ubiquitin-like protein. The Ub or Ubl is next transferred to a catalytic cysteine residue within the E1 to yield a thioester bond between the terminal glycine carboxyl group and the cysteine. Then the E1 transfers the Ub/Ubl to its partner E2 in an exchange reaction with the Ub/Ubl glycine forming a thioester coupling with a cysteine of the E2. Finally, one of a range of E3 ligases enables transfer of the Ub or Ubl to the target protein of interest, typically forming a covalent isopeptide bond with the epsilon amino group of a lysine on the protein. Given the above described mechanism, an inhibitor that effectively competes for the binding site region occupied by the nucleotide related species (ATP, acyladenylate, AMP) on the E1 enzyme, will disrupt the entire Ub or Ubl transfer process.

Targeting E1 activating enzymes thus provides an opportunity to affect numerous biochemical pathways that are involved in maintaining the integrity of cell division and cell signaling. As described, E1 activating enzymes function at the first step of ubiquitin and ubiquitin-like molecule conjugation pathways. As a result, inhibition of an E1 activating enzyme will prevent attachment of its corresponding Ub or Ubl to its target proteins, and thereby modulate the normal downstream biological consequences of the ubiquitin and ubiquitin-like molecule modification. E1 enzymes, as regulators of diverse cellular functions, are important therapeutic targets for the identification of novel approaches to treatment of diseases and disorders.

Nedd8, an ubiquitin-like protein, is activated by an E1 enzyme known as Nedd8-activating enzyme (APPBP1-Uba3) (NAE). The activated Nedd8 is transferred to a primary E2 associate (Ubcl2). The most well characterized substrate proteins for neddylation are the cullin family members. These proteins act as core scaffolds for the SCF ubiquitin E3 ligase complex which, in turn, are involved in critical regulation of proteins involved in the cell cycle (p27 and cyclin E), NFκB pathway based transcriptional regulation and signal transduction (IκBα), and oxygen regulation (HIF-1α). More recently, the well established tumor suppressor p53 and its regulatory E3 ligase, MDM2, have both been found to be conjugated with NEDD8 and thereby regulated as well. Thus, given the importance of neddylation in controlling critical cellular processes, the ability to modulate and inhibit the degree of neddylation of target proteins will be therapeutically useful. Moreover, inhibition of the neddylation process can be accomplished via inhibition of the Nedd8 activating enzyme. Therefore, inhibitors of Nedd8 activating enzyme (NAE) are important therapeutic targets; there exists a need for such compounds.

### SUMMARY OF THE INVENTION

In a broad aspect, the disclosure encompasses the compounds of formula (I), shown below, pharmaceutical compositions containing those compounds and methods employing such compounds or compositions in the treatment of diseases and/or disorders, such as cancer, inflammatory disorders, neurodegenerative disorders, inflammation associated with infection, cachexia, or the like.

Thus, one aspect of the disclosure provides compounds of formula (I):

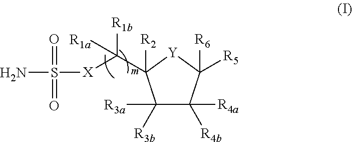

or pharmaceutically acceptable salts thereof, wherein
m is 1, 2, or 3;
X is —CH₂—, —CH(halogen)-, —C(halogen)₂-, —O—, —NH—, or —N(C₁-C₆ alkyl)-;
Y is —O—, —S—, or —CR₁₀R₁₁—;
R₁ₐ and R_{1b} are each independently hydrogen, halogen, C₁-C₄ aliphatic, or C₁-C₄ haloaliphatic, or R₁ₐ and R_{1b}, taken together with the carbon to which they are attached, form a C₃-C₈ cycloalkyl ring, or one of R₁ₐ and R_{1b}, taken together with R₂ and the intervening carbon atoms, forms a C₃-C₈ cycloalkyl ring, or R₁ₐ and R_{1b} together form ═O;
R₂ is hydrogen, or C₁-C₄ aliphatic, or R₂ together with one R₁ₐ or R_{1b} and the carbon atoms to which they are attached forms C₃-C₈ cycloalkyl;
R₃ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —C(O)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄), or R₃ₐ and R_{3b} together form ═O, or R₃ₐ and R₄ₐ together form a bond;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CONH₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄); or R₄ₐ and R_{4b} together form ═O, ═CH₂, ═CHF, or ═CF₂, or R₄ₐ and R₃ₐ together form a bond;
R_{4b} is selected from the group consisting of hydrogen, halogen, C₁-C₄ aliphatic, and C₁-C₄ haloaliphatic, or R₄ₐ and R_{4b} together form ═O, ═CH₂, ═CHF, or ═CF₂;
R₅ is a ring selected from the group consisting of:

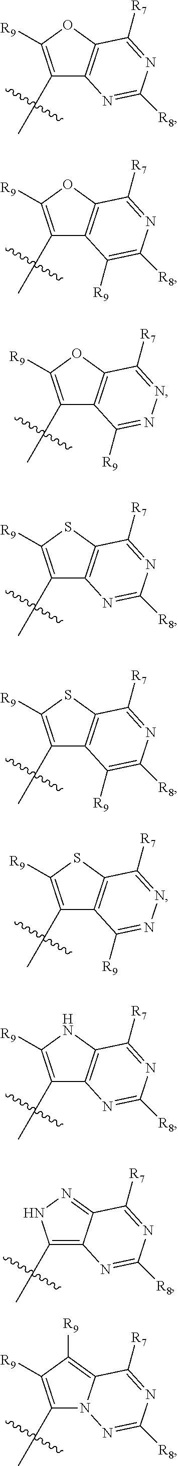

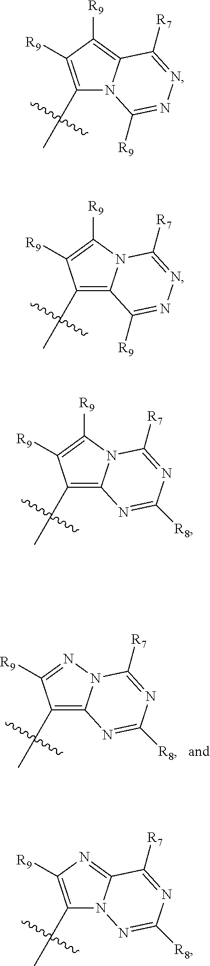

wherein one ring nitrogen atom is optionally oxidized;
R₆ is hydrogen, or C₁-C₄ aliphatic;
   wherein R₇ is independently selected from hydrogen, halogen, —CN, —OH, —OR₁₂, —SH, —SR₁₂, —NH₂, —NHR₁₂, —N(R₁₂)₂, and —R₁₅;
   each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
   each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
   each R₁₀ is are independently hydrogen, halogen, —OH, optionally substituted C₁-C₄ aliphatic, or C₁-C₄ haloaliphatic;
   each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
   each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
   each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
   each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
   R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
   wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
   Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
   R₁₆ₐ is optionally substituted aryl, optionally substituted heteroaryl, optionally substituted heterocycyl, or optionally substituted cycloaliphatic group;
   R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂, —C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
   R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
   R₁₇ is independently hydrogen, optionally substituted C₁-C₆ aliphatic, optionally substituted aryl, optionally substituted heteroaryl, or optionally substituted heterocyclyl;
   R₁₈ is independently hydrogen, optionally substituted C₁-C₆ aliphatic, optionally substituted aryl, optionally substituted heteroaryl, or optionally substituted heterocyclyl;
   R₁₉ is independently optionally substituted C₁-C₆ aliphatic, optionally substituted aryl, or optionally substituted heteroaryl; and
   R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

The disclosure also provides synthetic intermediates that are useful in making the compounds of formula (I).

The disclosure also provides methods of preparing compounds of the disclosure and the intermediates used in those methods.

The disclosure also provides pharmaceutical compositions comprising a compound of formula (I) or a pharmaceutically acceptable salt thereof and at least one pharmaceutically acceptable carrier, solvent, adjuvant or diluent.

The disclosure also provides methods for inhibiting E1 enzyme activity in vitro and in vivo comprising administering compounds of formula (I).

The disclosure also provides a method of treating a disease or disorder comprising administering compounds of formula (I). Examples of diseases or disorders include cancer, an inflammatory disorder, a neurodegenerative disorder, inflammation associated with infection, and cachexia.

The disclosure further provides a compound or pharmaceutical composition thereof in a kit with instructions for using the compound or composition.

The disclosure further provides compounds that may be administered alone or in combination with other drugs or therapies known to be effective to treat the disease to enhance overall effectiveness of therapy.

### DETAILED DESCRIPTION OF THE INVENTION

In one embodiment, the disclosure provides compounds of formula (I) wherein
m is 1, or 2;
X is —CH₂—, —CH(halogen)-, —O—, —NH—, or —N(C₁-C₆ alkyl)-;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₃ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CONH₂, —CON(H)R₁₂, —OC(O)N(H)R₁₂, —C(O)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄), or R₃ₐ and R_{3b} together form ═O, or R₃ₐ and R₄ₐ together form a bond;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄); or R₄ₐ and R_{4b} together form ═O, ═CH₂, ═CHF, or ═CF₂, or R₄ₐ and R₃ₐ together form a bond;
R_{4b} is selected from the group consisting of hydrogen, halogen, C₁-C₄ aliphatic, and C₁-C₄ haloaliphatic, or R₄ₐ and R_{4b} together form ═O, ═CH₂, ═CHF, or ═CF₂;
R₅ is a ring selected from the group consisting of:

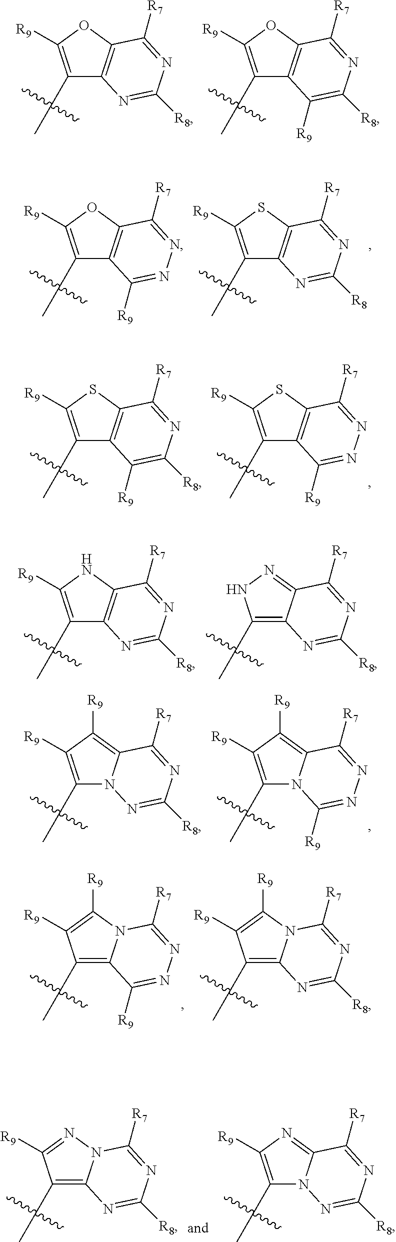

wherein one ring nitrogen atom is optionally oxidized;
R₆ is hydrogen, or C₁-C₄ aliphatic;
   wherein R₇ is independently selected from hydrogen, halogen, —CN, —OH, —OR₁₂, —SH, —SR₁₂, —NH₂, —NHR₁₂, —N(R₁₂)₂, and —R₁₅;
   each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
   each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
   each R₁₀ is are independently hydrogen, halogen, —OH, optionally substituted C₁-C₄ aliphatic, or C₁-C₄ haloaliphatic;
   each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
   each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
   each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
   each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
   R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
   wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
   Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
   R₁₆ₐ is optionally substituted aryl, optionally substituted heteroaryl, optionally substituted heterocycyl, or optionally substituted cycloaliphatic group;
   R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
   R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
   R₁₇ is independently hydrogen, optionally substituted C₁-C₆ aliphatic, optionally substituted aryl, optionally substituted heteroaryl, or optionally substituted heterocyclyl;
   R₁₈ is independently hydrogen, optionally substituted C₁-C₆ aliphatic, optionally substituted aryl, optionally substituted heteroaryl, or optionally substituted heterocyclyl;
   R₁₉ is independently optionally substituted C₁-C₆ aliphatic, optionally substituted aryl, or optionally substituted heteroaryl; and
   R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

In another embodiment, the disclosure provides compounds of formula (I) wherein
m is 1, or 2;
X is —CH₂—, —CH(halogen)-, —O—, —NH—, or —N(C₁-C₆ alkyl)-;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₁ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄), or R₃ₐ and R_{3b} together form ═O;
R_{3b} is selected from the group consisting of hydrogen, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄);
R_{4b} is selected from the group consisting of hydrogen, halogen, C₁-C₄ aliphatic, and C₁-C₄ haloaliphatic;
R₅ is a ring selected from the group consisting of:

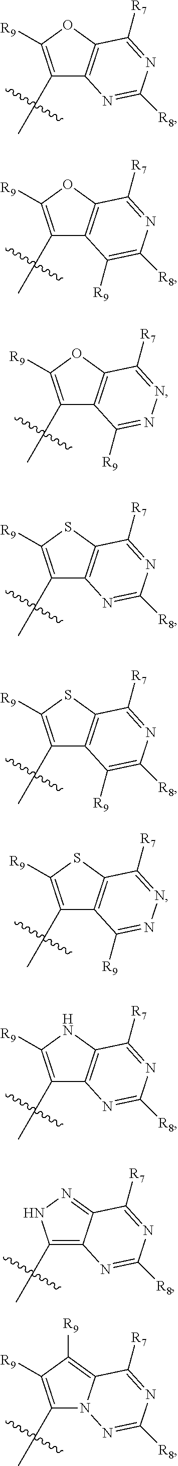

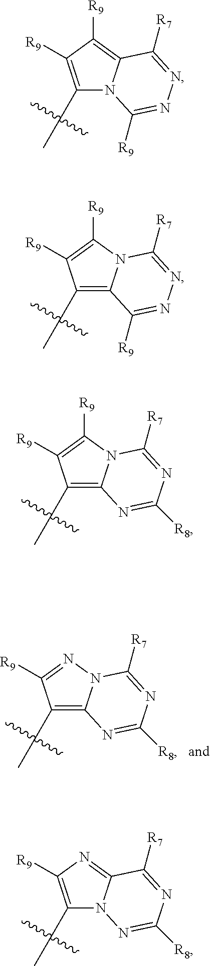

wherein one ring nitrogen atom is optionally oxidized;
R₆ is hydrogen, or C₁-C₄ aliphatic;
   wherein R₇ is independently selected from hydrogen, halogen, —CN, —OH, —OR₁₂, —SH, —SR₁₂, —NH₂, —NHR₁₂, —N(R₁₂)₂, and —R₁₅;
   each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
   each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
   each R₁₀ is are independently hydrogen, halogen, —OH, optionally substituted C₁-C₄ aliphatic, or C₁-C₄ haloaliphatic;
   each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
   each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
   each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
   each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
   R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
   wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
   Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
   R₁₆ₐ is optionally substituted aryl, optionally substituted heteroaryl, optionally substituted heterocycyl, or optionally substituted cycloaliphatic group;
   R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
   R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
   R₁₇ is independently hydrogen, optionally substituted C₁-C₆ aliphatic, optionally substituted aryl, optionally substituted heteroaryl, or optionally substituted heterocyclyl;
   R₁₈ is independently hydrogen, optionally substituted C₁-C₆ aliphatic, optionally substituted aryl, optionally substituted heteroaryl, or optionally substituted heterocyclyl;
   R₁₉ is independently optionally substituted C₁-C₆ aliphatic, optionally substituted aryl, or optionally substituted heteroaryl; and
   R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

In certain embodiments, the disclosure as described above provides compounds of formula (I) wherein
m is 1, or 2;
X is —CH₂—, —O—, or —NH—;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₃ₐ is selected from the group consisting of hydrogen, halogen, —CN, —OH, —OR₁₂, —NH₂, —NH(R₁₂), —N(H)CO₂R₁₂, —N(H)C(O)R₁₂, —OC(O)N(H)R₁₂, —OC(O)R₁₂, —OC(O)OR₁₂, —OS(O)₂NH₂, optionally substituted C₁-C₄ haloaliphatic, and C₁-C₄ aliphatic;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —OH, —OR₁₂, —NH₂, —NH(R₁₂), —N(H)CO₂R₁₂, —N(H)C(O)R₁₂, —OC(O)N(H)R₁₂, —OC(O)R₁₂, —OC(O)OR₁₂, optionally substituted C₁-C₄ haloaliphatic, and C₁-C₄ aliphatic;
R_{4b} is selected from the group consisting of hydrogen, halogen, C₁-C₄ aliphatic, and C₁-C₄ haloaliphatic, or R₄ₐ and R_{4b} together form ═O or ═CH₂;
R₅ is a ring selected from the group consisting of:

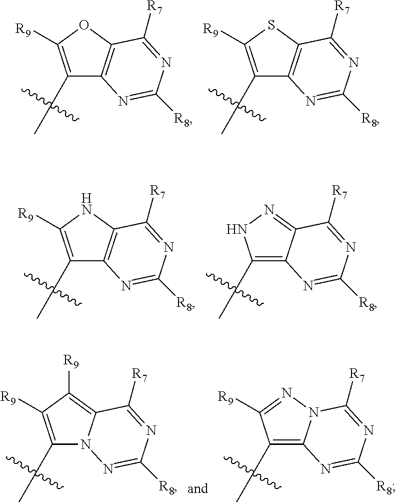

R₆ is hydrogen, or C₁-C₄ aliphatic;
   wherein R₇ is independently selected from halogen, —OH, —OR₁₂, —SR₁₂, —NH₂, —NHR₁₂, —N(R₁₂)₂, and —R₁₅;
   each R₈ is independently hydrogen, halogen, —CN, —OH, —OR₁₂, —SR₁₀, —NH₂, —NHR₁₀, —N(R₁₀)₂, or optionally substituted C₁-C₄ aliphatic group;
   each R₉ is independently selected from hydrogen, halogen, —CN, —OR₁₀, —SR₁₀, —N(R₁₀)(R₁₁), optionally substituted C₁-C₆ aliphatic;
   each R₁₀ is are independently hydrogen, halogen, —OH, optionally substituted C₁-C₄ aliphatic, or C₁-C₄ haloaliphatic;
   each R₁₁ is independently hydrogen, halogen, —OH, optionally substituted C₁-C₄ aliphatic, or C₁-C₄ haloaliphatic;
   each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
   R₁₅ is -V-Z-R₁₆ₐ, -V-Z-R_{16b}, —R_{16c}, or an optionally substituted aliphatic, aryl, heteroaryl, or heterocyclyl group;
   wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—/—N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
   Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
   R₁₆ₐ is optionally substituted aryl, optionally substituted heteroaryl, optionally substituted heterocycyl, or optionally substituted cycloaliphatic group;
   R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—OC₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
   R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—OC₂—R₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
   R₁₇ is independently hydrogen, optionally substituted C₁-C₆ aliphatic, optionally substituted aryl, optionally substituted heteroaryl, or optionally substituted heterocyclyl;
   R₁₈ is independently hydrogen, optionally substituted C₁-C₆ aliphatic, optionally substituted aryl, optionally substituted heteroaryl, or optionally substituted heterocyclyl;
   R₁₉ is independently optionally substituted C₁-C₆ aliphatic, optionally substituted aryl, or optionally substituted heteroaryl; and
   R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

In certain embodiments, the disclosure as described above provides compounds of formula (I) wherein m is 1.

In other embodiments, the disclosure as described above provides compounds of formula (I) wherein X is —O—, —CH₂—, or —NH—.

In another embodiment, the disclosure as described above provides compounds of formula (I) wherein X is —O—.

In certain embodiments, the disclosure as described above provides compounds of formula (I) wherein Y is —O—, or —CR₁₀R₁₁—.

In other embodiments, the disclosure as described above provides compounds of formula (I) wherein Y is —O—.

In other embodiments, the disclosure as described above provides compounds of formula (I) wherein Y is —CR₁₀R₁₁—. In some embodiments, R₁₀ and R₁₁ are independently hydrogen, halogen, —OH, or optionally substituted C₁-C₄ aliphatic. In some embodiments, R₁₀ and R₁₁ are independently hydrogen.

In certain embodiments, the disclosure as described above provides compounds of formula (I) wherein R₂ and R₆ are independently hydrogen.

In other embodiments, the disclosure as described above provides compounds of formula (I) wherein R₃ₐ is selected from the group consisting of hydrogen, halogen, —OH, —OR₁₂, —OC(O)N(H)R₁₂, —OC(O)R₁₂, —OC(O)OR₁₂, and —OS(O)₂NH₂, or R₃ₐ and R_{3b} together form ═O.

In another embodiment, the disclosure as described above provides compounds of formula (I) wherein R₃ₐ is selected from the group consisting of hydrogen, halogen, —OH, —OR₁₂, and —OS(O)₂NH₂, or R₃ₐ and R_{3b} together form ═O.

In yet another embodiment, the disclosure as described above provides compounds of formula (I) wherein R₃ₐ is selected from the group consisting of hydrogen, —OH, and —OS(O)₂NH₂, or R₃ₐ and R_{3b} together form ═O.

In certain embodiments, the disclosure as described above provides compounds of formula (I) wherein R₃ₐ is —OH.

In certain embodiments, the disclosure as described above provides compounds of formula (I) wherein R₃ₐ is —OS(O)₂NH₂.

In certain embodiments, the disclosure as described above provides compounds of formula (I) wherein R₃ₐ is hydrogen.

In other embodiments, the disclosure as described above provides compounds of formula (I) wherein R_{3b} is hydrogen.

In certain embodiments, the disclosure as described above provides compounds of formula (I) wherein R₃ₐ and R_{3b} together form ═O.

In certain embodiments, the disclosure as described above provides compounds of formula (I) wherein R₄ₐ is selected from the group consisting of hydrogen, halogen, —OH, —OR₁₂, and optionally substituted C₁-C₄ aliphatic.

In certain embodiments, the disclosure as described above provides compounds of formula (I) wherein R₄ₐ is —OH.

In other embodiments, the disclosure as described above provides compounds of formula (I) wherein R₄ₐ is hydrogen.

In certain embodiments, the disclosure as described above provides compounds of formula (I) wherein R_{4b} is hydrogen.

In certain embodiments, the disclosure as described above provides compounds of formula (I) wherein R₃ₐ is —OH, and R₄ₐ is hydrogen. In some embodiments, R_{3b} is hydrogen, and R_{4b} is hydrogen.

In certain embodiments, the disclosure as described above provides compounds of formula (I) wherein R₃ₐ is —OH, and R₄ₐ is —OH. In some embodiments, R_{3b} is hydrogen, and R_{4b} is hydrogen.

In certain embodiments, the disclosure as described above provides compounds of formula (I) wherein R₅ is selected from the group consisting of:

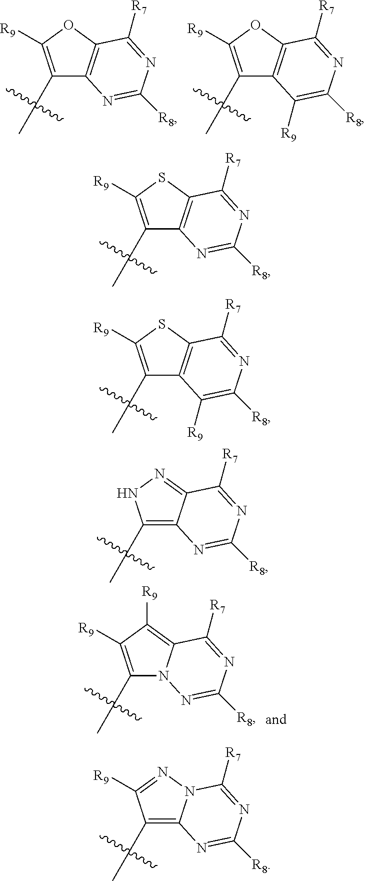

In certain embodiments, the disclosure as described above provides compounds of formula (I) wherein R₅ is selected from the group consisting of:

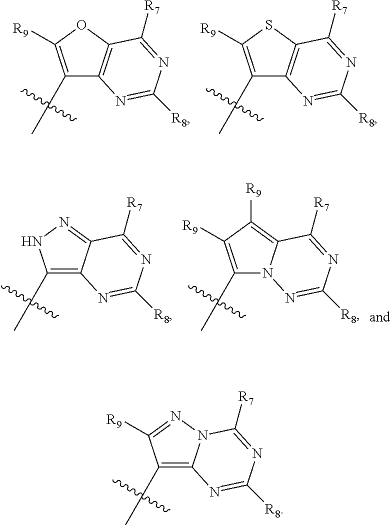

In certain embodiments, the disclosure as described above provides compounds of formula (I) wherein R₇ is —NH₂, —NHR₁₂, or —R₁₅;

each R₁₂ is independently an optionally substituted C₁-C₁₀ aliphatic, optionally substituted aryl, optionally substituted C₃-C₁₄ cycloaliphatic, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In other embodiments, the disclosure as described above provides compounds of formula (I) wherein R₇ is —NH₂, or —NHR₁₂; and

each R₁₂ is independently an optionally substituted C₁-C₁₀ aliphatic, optionally substituted aryl, optionally substituted C₃-C₁₄ cycloaliphatic, optionally substituted heteroaryl, or optionally substituted heterocyclyl group. In some embodiments, R₁₂ is optionally substituted aryl or optionally substituted C₃-C₁₄ cycloaliphatic. In some embodiments, R₁₂ is optionally substituted C₆-C₁₀ cycloaliphatic. In other embodiments, R₁₂ is optionally substituted C₉-C₁₀ cycloaliphatic. In some embodiments, R₁₂ is optionally substituted 2,3-dihydro-1H-inden-1-yl.

In certain embodiments, the disclosure as described above provides compounds of formula (I) wherein R₇ is —R₁₅; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In certain embodiments, the disclosure as described above provides compounds of formula (I) wherein R₇ is —R₁₅; and
R₁₅ is -V-Z-R_{16d};
   where V is —NH—,
   Z is an optionally substituted C₁-C₆ alkylene chain, and
   R_{16d} is optionally substituted aryl, optionally substituted heteroaryl, optionally substituted heterocyclyl, or optionally substituted cycloaliphatic group.

In other embodiments, the disclosure as described above provides compounds of formula (I) wherein R₇ is —NH—CH₂—R_{16d}, where R_{16d} is phenyl optionally substituted with one or more of halogen, —NO₂, —CN, —OH, —NH₂, —NH(C₁-C₆ alkyl), —N(C₁-C₆ alkyl)₂, C₁-C₆ alkyl, and C₁-C₆ haloalkyl.

In certain embodiments, the disclosure as described above provides compounds of formula (I) wherein R₅ is:

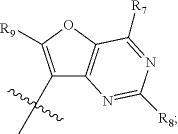

and
R₇ is —NH₂, —NHR₁₂, or —R₁₅;
   each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In certain embodiments, the disclosure as described above provides compounds of formula (I) wherein R₅ is:

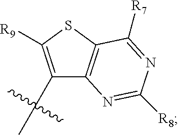

and
R₇ is —NH₂, —NHR₁₂, or —R₁₅;
   each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In certain embodiments, the disclosure as described above provides compounds of formula (I) wherein R₅ is:

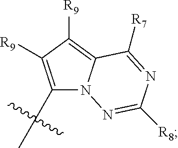

and
R₇ is —NH₂, —NHR₁₂, or —R₁₅;
   each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In certain embodiments, the disclosure as described above provides compounds of formula (I) wherein R₅ is:

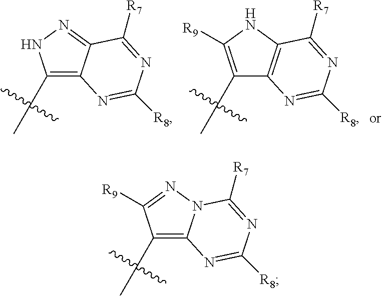

and
R₇ is —NH₂, —NHR₁₂, or —R₁₅;
   each R₁₂ is independently an optionally substituted C₁-C₁₀, aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In other embodiments, the disclosure as described above provides compounds of formula (I) wherein R₅ is:

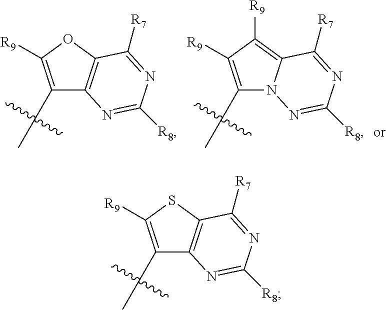

R₇ is —NH₂, —NHR₁₂, or —R₁₅;
   each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In other embodiments, the disclosure as described above provides compounds of formula (I) wherein R₅ is:

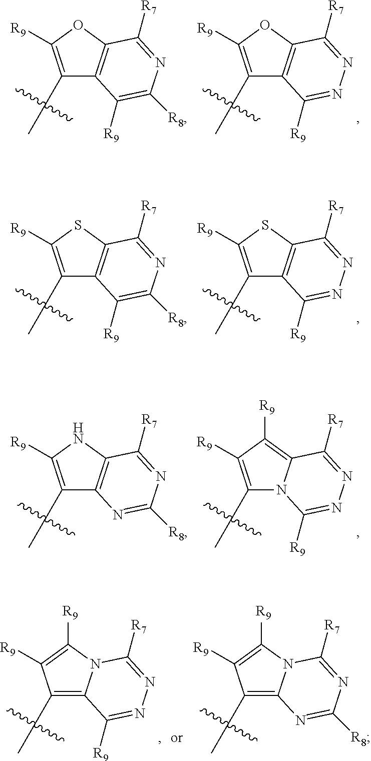

R₇ is —NH₂, —NHR₁₂, or —R₁₅;
   each R₁₂ is independently an optionally substituted C₁-C₄ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and

R_{16d} is halogen, —NO₂, —CN, —OR₁₈, —SR₁₉, —N(R₂₀)₂, optionally substituted aryl, optionally substituted heteroaryl, optionally substituted heterocycyl, or optionally substituted cycloaliphatic group.

In certain embodiments, the disclosure as described above provides compounds of formula (I) wherein R₇ is —NH₂, —NHR₁₂, or —R₁₅;

each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In other embodiments, the disclosure as described above provides compounds of formula (I) wherein R₇ is —NH₂, or —NHR₁₂; and

each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group. In some embodiments, R₁₂ is optionally substituted aryl or optionally substituted C₃-C₁₄ cycloaliphatic. In some embodiments, R₁₂ is optionally substituted C₆-C₁₀ cycloaliphatic. In other embodiments, R₁₂ is optionally substituted C₉-C₁₀ cycloaliphatic. In some embodiments, R₁₂ is optionally substituted 2,3-dihydro-1H-inden-1-yl.

In certain embodiments, the disclosure as described above provides compounds of formula (I) wherein R₇ is —R₁₅; and
R₁₅ is -V-Z-R_{16d};
   where V is —N(R₁₇)—,
   Z is an optionally substituted C₁-C₆ alkylene chain, and
   R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

In certain embodiments, the disclosure as described above provides compounds of formula (I) wherein R₇ is —R₁₅; and
R₁₅ is -V-Z-R_{16d};
   where V is —NH—,
   Z is an optionally substituted C₁-C₆ alkylene chain, and
   R_{16d} is optionally substituted aryl, optionally substituted heteroaryl, optionally substituted heterocycyl, or optionally substituted cycloaliphatic group.

In other embodiments, the disclosure as described above provides compounds of formula (I) wherein R₇ is —NH—CH₂—R_{16d}, where R_{16d} is phenyl optionally substituted with one or more of halogen, —NO₂, —CN, —OH, —NH₂, —NH(C₁-C₆ alkyl), —N(C₁-C₆ alkyl)₂, C₁-C₆ alkyl, and C₁-C₆ haloalkyl.

In certain embodiments, the disclosure as described above provides compounds of formula (I) wherein the substituents R₂, R₃ₐ, R_{3b}, R₄ₐ, R₄ₐ, and R₅ have the following stereochemical configuration when Y is —O—:

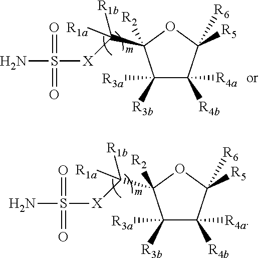

In other embodiments, the stereochemical configuration is:

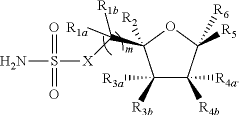

In certain embodiments, the disclosure as described above provides compounds of formula (I) wherein the substituents R₂, R₃ₐ, R_{3b}, R₄ₐ, R₄ₐ, and R₅ have the following stereochemical configuration when Y is —CH₂—:

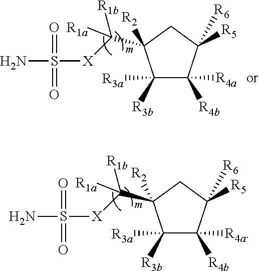

In other embodiments, the stereochemical configuration is:

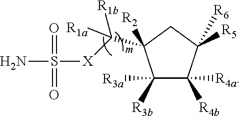

Representative compounds of the invention include:
(5-(4-(2,3-dihydro-1H-inden-1-ylamino)pyrrolo[1,2-f][1,2,4]triazin-7-yl)-3-oxotetrahydrofuran-2-yl)methyl sulfamate;
(5-(4-(2,3-dihydro-1H-inden-1-ylamino)pyrrolo[1,2-f][1,2,4]triazin-7-yl)-3-hydroxytetrahydrofuran-2-yl)methyl sulfamate;
(5-(7-amino-2H-pyrazolo[4,3-d]pyrimidin-3-yl)-3,4-dihydroxytetrahydrofuran-2-yl)methyl sulfamate;
(5-(4-aminothieno[3,2-d]pyrimidin-7-yl)-3,4-dihydroxytetrahydrofuran-2-yl)methyl sulfamate;
(5-(4-(2,3-dihydro-1H-inden-1-ylamino)thieno[3,2-d]pyrimidin-7-yl)-3,4-dihydroxytetrahydrofuran-2-yl)methyl sulfamate;
(5-(4-(4-chlorobenzylamino)thieno[3,2-d]pyrimidin-7-yl)-3,4-dihydroxytetrahydrofuran-2-yl)methyl sulfamate;
(4-(4-(2,3-dihydro-1H-inden-1-ylamino)pyrrolo[1,2-f][1,2,4]triazin-7-yl)-2-hydroxycyclopentyl)methyl sulfamate;
(4-(4-(2,3-dihydro-1H-inden-1-ylamino)pyrrolo[1,2-f][1,2,4]triazin-7-yl)-2-hydroxycyclopentyl)methyl sulfamate;
(4-(4-(2,3-dihydro-1H-inden-1-ylamino)thieno[3,2-d]pyrimidin-7-yl)-2-hydroxycyclopentyl)methyl sulfamate;
(5-(4-(2,3-dihydro-1H-inden-1-ylamino)thieno[3,2-d]pyrimidin-7-yl)-3-hydroxytetrahydrofuran-2-yl)methyl sulfamate;
(5-(4-aminofuro[3,2-d]pyrimidin-7-yl)-3,4-dihydroxytetrahydrofuran-2-yl)methyl sulfamate;
(5-(4-(2,3-dihydro-1H-inden-1-ylamino)furo[3,2-d]pyrimidin-7-yl)-3,4-dihydroxytetrahydrofuran-2-yl)methyl sulfamate;
(5-(4-aminopyrazolo[1,5-a][1,3,5]triazin-8-yl)-3,4-dihydroxytetrahydrofuran-2-yl)methyl sulfamate;
(5-(4-(2,3-dihydro-1H-inden-1-ylamino)pyrazolo[1,5-a][1,3,5]triazin-8-yl)-3,4-dihydroxytetrahydrofuran-2-yl)methyl sulfamate;
(4-(4-(2,3-dihydro-1H-inden-1-ylamino)pyrazolo[1,5-a][1,3,5]triazin-8-yl)-2-hydroxycyclopentyl)methyl sulfamate;
(5-(4-(2,3-dihydro-1H-inden-1-ylamino)-5H-pyrrolo[3,2-d]pyrimidin-7-yl)-3,4-dihydroxytetrahydrofuran-2-yl)methyl sulfamate;
(4-(4-(2,3-dihydro-1H-inden-1-ylamino)furo[3,2-d]pyrimidin-7-yl)-2-hydroxycyclopentyl)methyl sulfamate;
(4-(4-(cyclohexylmethylamino)thieno[3,2-d]pyrimidin-7-yl)-2-hydroxycyclopentyl)methyl sulfamate;
[2-[(aminosulfonyl)oxy]-4-{4-[2,3-dihydro-1H-inden-1-ylamino]pyrrolo[2,1-f][1,2,4]triazin-7-yl}cyclopentyl]methyl sulfamate;
(4-(4-(2,3-dihydro-1H-inden-1-ylamino)pyrrolo[1,2-f][1,2,4]triazin-7-yl)-2-hydroxycyclopentyl)methyl sulfamate;
[2-[(aminosulfonyl)oxy]-4-{4-[2,3-dihydro-1H-inden-1-ylamino]pyrazolo[1,5-a][1,3,5]triazin-8-yl}cyclopentyl]methyl sulfamate;
(4-(4-(2,3-dihydro-1H-inden-1-ylamino)thieno[3,2-d]pyrimidin-7-yl)-2-hydroxycyclopentyl)methyl sulfamate;
(4-(4-(2,3-dihydro-1H-inden-1-ylamino)thieno[3,2-d]pyrimidin-7-yl)-2-hydroxycyclopentyl)methyl sulfamate;
(5-(4-(2,3-dihydro-1H-inden-1-ylamino)thieno[3,2-d]pyrimidin-7-yl)-3,4-dihydroxytetrahydrofuran-2-yl)methyl sulfamate;
(5-(4-(benzylamino)thieno[3,2-d]pyrimidin-7-yl)-3,4-dihydroxytetrahydrofuran-2-yl)methyl sulfamate;
(5-(4-(cyclopropylmethylamino)thieno[3,2-d]pyrimidin-7-yl)-3,4-dihydroxytetrahydrofuran-2-yl)methyl sulfamate;
(5-(4-(cyclopropylamino)thieno[3,2-d]pyrimidin-7-yl)-3,4-dihydroxytetrahydrofuran-2-yl)methyl sulfamate;
(3,4-dihydroxy-5-(4-(2-methoxyethylamino)thieno[3,2-d]pyrimidin-7-yl)tetrahydrofuran-2-yl)methyl sulfamate;
(5-(4-(2,3-dihydro-1H-inden-1-ylamino)thieno[3,2-d]pyrimidin-7-yl)-3,4-dihydroxytetrahydrofuran-2-yl)methyl sulfamate;
(5-(4-(2-chlorobenzylamino)thieno[3,2-d]pyrimidin-7-yl)-3,4-dihydroxytetrahydrofuran-2-yl)methyl sulfamate;
(5-(4-(4-chlorobenzylamino)pyrazolo[1,5-a][1,3,5]triazin-8-yl)-3,4-dihydroxytetrahydrofuran-2-yl)methyl sulfamate;
(5-(4-(2,3-dihydro-1H-inden-1-ylamino)pyrazolo[1,5-a][1,3,5]triazin-8-yl)-3,4-dihydroxytetrahydrofuran-2-yl)methyl sulfamate;
(5-(4-(2,3-dihydro-1H-inden-1-ylamino)furo[3,2-d]pyrimidin-7-yl)-3,4-dihydroxytetrahydrofuran-2-yl)methyl sulfamate;
(5-(4-(2,4-dichlorobenzylamino)furo[3,2-d]pyrimidin-7-yl)-3,4-dihydroxytetrahydrofuran-2-yl)methyl sulfamate;
(5-(4-(4-chloro-3-(trifluoromethyl)benzylamino)furo[3,2-d]pyrimidin-7-yl)-3,4-dihydroxytetrahydrofuran-2-yl)methyl sulfamate;
(3,4-dihydroxy-5-(4-(2-hydroxy-2,3-dihydro-1H-inden-1-ylamino)furo[3,2-d]pyrimidin-7-yl)tetrahydrofuran-2-yl)methyl sulfamate;
(5-(4-(3-chlorobenzylamino)thieno[3,2-d]pyrimidin-7-yl)-3,4-dihydroxytetrahydrofuran-2-yl)methyl sulfamate;
(4-(4-(2,3-dihydro-1H-inden-1-ylamino)furo[3,2-d]pyrimidin-7-yl)-2-hydroxycyclopentyl)methyl sulfamate;
(4-(4-(5-fluoro-2,3-dihydro-1H-inden-1-ylamino)thieno[3,2-d]pyrimidin-7-yl)-2-hydroxycyclopentyl)methyl sulfamate;
(2-hydroxy-4-(4-(5-methyl-2,3-dihydro-1H-inden-1-ylamino)thieno[3,2-d]pyrimidin-7-yl)cyclopentyl)methyl sulfamate;
(4-(4-(4-bromo-2,3-dihydro-1H-inden-1-ylamino)thieno[3,2-d]pyrimidin-7-yl)-2-hydroxycyclopentyl)methyl sulfamate;
(2-hydroxy-4-(4-(1,2,3,4-tetrahydronaphthalen-1-ylamino)thieno[3,2-d]pyrimidin-7-yl)cyclopentyl)methyl sulfamate;
(2-hydroxy-4-(4-(1,2,3,4-tetrahydronaphthalen-1-ylamino)thieno[3,2-d]pyrimidin-7-yl)cyclopentyl)methyl sulfamate;
(2-hydroxy-4-(4-(4-methyl-2,3-dihydro-1H-inden-1-ylamino)thieno[3,2-d]pyrimidin-7-yl)cyclopentyl)methyl sulfamate;
(2-hydroxy-4-(4-(6-methoxy-2,3-dihydro-1H-inden-1-ylamino)thieno[3,2-d]pyrimidin-7-yl)cyclopentyl)methyl sulfamate;
(4-(4-(6,7-dihydro-5H-indeno[5,6-d][1,3]dioxol-5-ylamino)thieno[3,2-d]pyrimidin-7-yl)-2-hydroxycyclopentyl)methyl sulfamate;
(2-hydroxy-4-(4-(4-methoxy-2,3-dihydro-1H-inden-1-ylamino)thieno[3,2-d]pyrimidin-7-yl)cyclopentyl)methyl sulfamate;
(4-(4-(2,3-dihydro-1H-inden-1-ylamino)thieno[3,2-d]pyrimidin-7-yl)-2-hydroxycyclopentyl)methyl sulfamate;
(4-(4-(4-chlorobenzylamino)thieno[3,2-d]pyrimidin-7-yl)-2-hydroxycyclopentyl)methyl sulfamate;
(4-(4-((2,3-dihydro-1H-inden-1-yl)(sulfamoyl)amino)thieno[3,2-d]pyrimidin-7-yl)-2-hydroxycyclopentyl)methyl sulfamate;
(4-(4-(cyclohexylmethylamino)thieno[3,2-d]pyrimidin-7-yl)-2-hydroxycyclopentyl)methyl sulfamate;
(4-(4-(4-bromo-2,3-dihydro-1H-inden-1-ylamino)thieno[3,2-d]pyrimidin-7-yl)-2-hydroxycyclopentyl)methyl sulfamate;
(2-hydroxy-4-(4-(4-phenyl-2,3-dihydro-1H-inden-1-ylamino)thieno[3,2-d]pyrimidin-7-yl)cyclopentyl)methyl sulfamate;
(4-(4-(1-cyclohexylethylamino)thieno[3,2-d]pyrimidin-7-yl)-2-hydroxycyclopentyl)methyl sulfamate;
(2-hydroxy-4-(4-((5-methylfuran-2-yl)methylamino)thieno[3,2-d]pyrimidin-7-yl)cyclopentyl)methyl sulfamate;
(4-(4-(cyclopentylmethylamino)thieno[3,2-d]pyrimidin-7-yl)-2-hydroxycyclopentyl)methyl sulfamate;
(4-(4-(hexylamino)thieno[3,2-d]pyrimidin-7-yl)-2-hydroxycyclopentyl)methyl sulfamate; and
(4-(4-aminothieno[3,2-d]pyrimidin-7-yl)-2-hydroxycyclopentyl)methyl sulfamate.

### Therapeutics Applications

One aspect of the disclosure relates to inhibiting or decreasing E1 enzyme activity in a sample, either in vitro or in vivo, utilizing a compound or pharmaceutical composition described in this disclosure. The sample may be in one of many forms. Examples of the sample, as used herein, include, without limitation E1 enzyme in a purified sample, in a partially purified sample, in cultured cells, in cellular extracts, in biopsied cells and extracts thereof, in bodily fluids (e.g. blood, serum, urine, feces, saliva, semen, tears) and extracts thereof.

Another embodiment of the disclosure provides for treating a patient by administering to the patient a compound or pharmaceutical composition described in the disclosure. The treated patient may have a disorder, show symptoms of a disorder, or be at risk of developing a disorder or recurrence of a disorder. Treatment of the patient can cure, remedy, or heal the patient of the disorder. Alternatively, treatment of the patient can prevent, alleviate, diminish, palliate, or improve the disorder. Alternatively, treatment of the patient can affect or alter the symptoms of the disorder or predisposition toward the disorder. The disorders that can be treated are those disorders in which inhibition of E1 enzyme activity inhibits progression of the disorder. For example, diseased cells or tissues can be directly killed or inhibited as a result of E1 enzyme inhibition. Alternatively, E1 enzyme inhibition can lead to stabilization of proteins that in turn kill or inhibit diseased cells or tissues. Alternatively, E1 enzyme inhibition can inhibit the ability of other proteins to activate diseased cells or tissues.

Relevant, non-limiting, disorders that can be treated by administering to the patient a compound or pharmaceutical composition described in the disclosure include: proliferative diseases, especially cancers and inflammatory disorders (e.g. rheumatoid arthritis, inflammatory bowel disease, asthma, chronic obstructive pulmonary disease, osteoarthritis, dermatosis such as atopic dermatitis and psoriasis, vascular proliferative disorders such as atherosclerosis and restenosis); autoimmune diseases e.g. multiple sclerosis, tissue and organ rejection; inflammation associated with infection (e.g. immune responses); neurodegenerative disorders (e.g. Alzheimer's disease, Parkinson's disease, motor neurone disease, neuropathic pain, triplet repeat disorders, astrocytoma, and neurodegeneration as result of alcoholic liver disease); and ischemic injury (e.g. stroke). Additionally, applicable disorders also include any disorders that are dependent on functional cullin and/or ubiquitination activity (e.g. the ubiquitin proteasome pathway) and whose activity can be regulated by inhibiting E1 enzyme activity (e.g. NAE, UAE, SAE activity). Applicable disorders also include those in which the existence, maintenance, or progression of the disorder is mediated via p53 activation, NFκB activation, p27Kip activation, or p21 WAF/CIP1 activation.

The compounds and pharmaceutical compositions described in the disclosure are particularly useful for the treatment of cancer. Cancer, as defined herein, describes a disorder characterized by uncontrolled or deregulated cellular proliferation, abnormal cellular differentiation, an abnormal ability to invade surrounding tissue, and an inappropriate ability to establish new growth at ectopic sites. Cancer, as defined herein, refers to both primary and metastatic cancers. Treatable cancers include both solid tumors and hematologic (blood) tumor types, and the tumors may occur anywhere in the body including skin, tissues, organs, bone, cartilage, blood, and vessels. Treatable cancers can be in either adults or children.

Non-limiting examples of cancers that can be treated by the compounds and pharmaceutical compositions described in the disclosure include acute lymphoblastic leukemia, acute myeloid leukemia, adrenocortical carcinoma, AIDS-related cancers, anal cancer, appendix cancer, astrocytomas, atypical teratoid/rhabdoid tumor, basal cell carcinoma, bile duct cancer, bladder cancer, breast cancer, bronchial tumors, Burkitt lymphoma, carcinoid tumors, cervical cancer, chordoma, chronic lymphocytic leukemia, chronic myelogenous leukemia, chronic myeloproliferative disorders, colon cancer, colorectal cancer, craniopharyngioma, cutaneous t-cell lymphoma, endometrial cancer, ependymoblastoma, ependymoma, esophageal cancer, esthesioneuroblastoma, Ewing sarcoma family of tumors, extracranial germ cell tumor, extragonadal germ cell tumor, gallbladder cancer, gastric (stomach) cancer, gastrointestinal stromal tumor (GIST), gestational trophoblastic tumor, glioma (adult), glioma (childhood brain stem), hairy cell leukemia, head and neck cancer, heart cancer, hepatocellular (liver) cancer, Hodgkin lymphoma, hypopharyngeal cancer, intraocular melanoma, islet cell tumors, Kaposi sarcoma, kidney (renal cell) cancer, Langerhans cell histiocytosis, laryngeal cancer, lip and oral cavity cancer, liver cancer, medulloblastoma, medulloepithelioma, melanoma, Merkel cell carcinoma, mesothelioma, metastatic squamous neck cancer with occult primary, mouth cancer, multiple endocrine neoplasia syndromes, multiple myeloma, mycosis fungoides, myelodysplastic/myeloproliferative neoplasms, nasal cavity and paranasal sinus cancer, nasopharyngeal cancer, neuroblastoma, non-Hodgkin lymphoma, non-small cell lung cancer, oral cancer, oropharyngeal cancer, osteosarcoma and malignant fibrous histiocytoma of bone, ovarian cancer, ovarian epithelial cancer, ovarian germ cell tumor, pancreatic cancer, papillomatosis, parathyroid cancer, penile cancer, pharyngeal cancer, pineal parenchymal tumors, pineoblastoma, pituitary tumor, pleuropulmonary blastoma, primary central nervous system (CNS) lymphoma, prostate cancer, rectal cancer, renal cell (kidney) cancer, retinoblastoma, rhabdomyosarcoma, salivary gland cancer, Sézary syndrome, small cell lung cancer, small intestine cancer, soft tissue sarcoma, squamous cell carcinoma, stomach (gastric) cancer, supratentorial primitive neuroectodermal tumors, testicular cancer, throat cancer, thymoma and thymic carcinoma, thyroid cancer, transitional cell cancer of the renal pelvis and ureter, urethral cancer, uterine sarcoma, vaginal cancer, vulvar cancer, Waldenström macroglobulinemia, and Wilms tumor.

In some embodiments, the compound or pharmaceutical composition described in the disclosure is used to treat a patient either experiencing or being at risk of developing a recurrence in a cancer selected from the group consisting of breast cancer, colorectal cancer, gastric cancer, a hematologic cancer, lung cancer, ovarian cancer, pancreatic cancer, and prostate cancer. In certain preferred embodiments, the cancer is selected from the group consisting of colorectal cancer, a hematologic cancer, lung cancer, and ovarian cancer.

In some embodiments and for certain disorders, the E1 enzyme inhibitor described in the disclosure is used to treat the disorder in combination with another therapeutic agent already approved or recognized by appropriate governing authorities as suitable for treatment of the disorder. The E1 inhibitor of the disclosure may be administered in dosage form either separately or in a single combined dosage with the other therapeutic. When the E1 enzyme inhibitor of the disclosure and other agent are administered separately, they may be administered simultaneously or the E1 inhibitor may be administered first or the other therapeutic agent may be administered first.

In some embodiments, the disorder is a proliferative disease or cancer, and the E1 enzyme inhibitor of the disclosure is administered in a non-limiting combination with either radiotherapy or another therapeutic agent (e.g. cytoxic agent, chemotherapy agent, or immunotherapy) already approved or recognized by appropriate governing authorities as suitable for treatment of the disorder. Non-limiting examples of these agents include alkylating agents, antibiotics, antibodies, antimetabolites, kinase inhibitors, topoisomerase inhibitors, proteosome inhibitors taxanes, and vaccines. Non-limiting examples of such agents are aldesleukin, alemtuzumab, aminolevulinic acid, anastrozole, aprepitant, arsenic trioxide, azacitidine, bendamustine, bevacizumab, bexarotene, bortezomib, bleomycin, cabazitaxel, capecitabine, carboplatin, cetuximab, cisplatin, clofarabine, cyclophosphamide, cytarabine, dacarbazine, dasatinib, daunorubicin, decitabine, degarelix, denileukin diftitox, dexrazoxane, docetaxel, doxorubicin, eltrombopag olamine, epirubicin, erlotinib, etoposide, everolimus, exemestane, filgrastim, fludarabine, fluorouracil, fulvestrant, gefitinib, gemcitabine, gemtuzumab, ibritumomab, imatinib, imiquimod, irinotecan, ixabepilone, lapatinib, lenalidomide, letrozole, leucovorin, leuprolide, methotrexate, nelarabine, nilotinib, ofatumumab, oxaliplatin, paclitaxel, palifermin, palonosetron, panitumumab, pazopanib, pegaspargase, pemetrexed, plerixafor, pralatrexate, prednisone, procarbazine, paloxifene, rasburicase, recombinant HPV vaccine, rituximab, romidepsin, romiplostim, sipuleucel-T, sorafenib, sunitinib, tamoxifen, temozolomide, temsirolimus, thalidomide, topotecan, toremifene, tositumomab, trastuzumab, vincristine, vorinostat, and zoledronic acid.

In other embodiments, the E1 enzyme inhibitor of the disclosure may be combined with anti-inflammatory agents. Non-limiting examples of these agents include NSAID's, corticosteroids, methotrexate, hydroxychloroquine, sulfasalazine, leflunomide, TNF Inhibitors (e.g. etanercept and infliximab), T-cell costimulatory blocking agents (e.g. abatacept), B-cell depleting agents (e.g. rituximab), IL-1 receptor antagonists (e.g. anakinra), and intramuscular gold. The E1 enzyme inhibitor of the disclosure may also be combined with immunomodulatory and suppressive agents including azathioprine, cyclophosphamide, and cyclosporine A, siroliumus, and tacrolimus. The E1 enzyme inhibitor of the disclosure may also be combined with anti-bacterial and anti-viral agents.

### Pharmaceutical Compositions

In another aspect, the present disclosure provides compositions comprising one or more of compounds as described above with respect to formula (I) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For buccal administration, the compositions may take the form of tablets or lozenges formulated in conventional manner.

For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective local dosages without undue experimentation.

### DEFINITIONS

The following terms and expressions used herein have the indicated meanings.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “aliphatic”, as used herein, means straight-chain, branched or cyclic C₁-C₁₂ hydrocarbon which is completely saturated or which contains one or more units of unsaturation, but which is not aromatic. For example, suitable aliphatic groups include substituted or unsubstituted linear, branched or cyclic alkyl, alkenyl, alkynyl groups and hybrids thereof, such as cycloalkyl, (cylcoalkyl)alkyl, (cycloalkenyl)alkyl or (cycloalkyl)alkenyl. In various embodiments, the aliphatic group has one to ten carbon atoms. In one embodiment, the aliphatic group has one to eight carbon atoms. In another embodiment, the aliphatic group has one to six carbon atoms. In another embodiment, the aliphatic group has one to four carbon atoms.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

An “aralkyl” or “arylalkyl” group comprises an aryl group covalently attached to an alkyl group, either of which independently is optionally substituted. Preferably, the aralkyl group is aryl(C₁-C₆)alkyl, including, without limitation, benzyl, phenethyl, and naphthyl methyl.

The terms “cyano” and “nitrile” as used herein, mean a —CN group.

The term “cycloaliphatic”, used alone or as part of a larger moiety, refers to a saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Non-limiting examples include cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8 members.

In some embodiments, two adjacent substituents on a cycloaliphatic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the term “cycloaliphatic” includes aliphatic rings that are fused to one or more aryl, heteroaryl, or heterocyclyl rings. Nonlimiting examples include indanyl, 2,3-dihydro-1H-inden-1-yl, 5,6,7,8-tetrahydroquinoxalinyl, decahydronaphthyl, or tetrahydronaphthyl, where the radical or point of attachment is on the aliphatic ring.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The term “heterocyclyl” as used herein, means a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl(thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “nitro” as used herein, means a —NO₂ group.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

An aryl (including the aryl moiety in aralkyl, aralkoxy, aryloxyalkyl and the like) or heteroaryl (including the heteroaryl moiety in heteroaralkyl and heteroaralkoxy and the like) group may contain one or more substituents. Examples of suitable substituents on the unsaturated carbon atom of an aryl or heteroaryl group include -halo, —NO₂, —CN, —R′, —C(R′)═C(R′)₂, —C≡C—R′, —OR′, —SR″, —S(O)R′″, —SO₂R″, —SO₂N(R″)₂, —N(R″)₂, —NR″C(O)R′, —NR″C(O)N(R″)₂, —NR″CO₂R″, —O—CO₂R″, —OC(O)N(R″)₂, —C—C(O)R′, —CO₂R′, —C(O)—C(O)R′, —C(O)R′, —C(O)N(R″)₂, —C(═NR″)—N(R″)₂, —C(═NR″)—OR′, —N(R″)—N(R″)₂, —N(R″)C(—NR″)—N(R″)₂, —NR″SO₂R″, —NR″SO₂N(R″), —P(O)(R′)₂, —P(O)(R′)₂, —O—P(O)—OR′, and —P(O)(NR″)—N(R″)₂, wherein R″ is an optionally substituted aliphatic or aryl group, and R′ and R″ are as defined above, or two adjacent substituents, taken together with their intervening atoms, form a 5- to 6-membered unsaturated or partially unsaturated ring having 0-3 ring atoms selected from the group consisting of N, O, and S.

An aliphatic group or a non-aromatic heterocyclic ring may be substituted with one or more substituents. Examples of suitable substituents on the saturated carbon of an aliphatic group or of a non-aromatic heterocyclic ring include, without limitation, those listed above for the unsaturated carbon of an aryl or heteroaryl group and the following: ═O, ═S, ═C(R)₂, ═N—N(R″)₂, ═N—OR′, ═N—NHC(O)R′, ═N—NHCO₂R′″, ═N—NHCO₂R′″, or ═N—R′ where each R′, R″, and R′″ is as defined above. For the purposes of clarity, the term “substituted aliphatic” refers to an aliphatic group having at least one non-aliphatic substituent.

Suitable substituents on a substitutable nitrogen atom of a heteroaryl or heterocyclic ring include —R′, —N(R′)₂, —C(O)R′, —CO₂R′, —C(O)—C(O)R′, —C(O)CH₂C(O)R′, —SO₂R′, —SO₂N(R)₂, —C(═S)N(R′)₂, —C(═NH)—N(R′)₂, and —NR′SO₂R′; wherein each R′ is as defined above.

The term “thia” as used herein means a ═S group.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. By way of example, the compounds of formula (I) wherein R^{3a} is hydroxy can have an R or S configuration at the carbon atom bearing R^{3a}. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Modulating” or “modulate” refers to the treating, prevention, suppression, enhancement or induction of a function, condition or disorder. For example, it is believed that the compounds of the present disclosure can modulate atherosclerosis by stimulating the removal of cholesterol from atherosclerotic lesions in a human.

“Treating” or “treatment” as used herein covers the treatment of a disease or disorder described herein, in a subject, preferably a human, and includes:

i. inhibiting a disease or disorder, i.e., arresting its development;

ii. relieving a disease or disorder, i.e., causing regression of the disorder;

iii. slowing progression of the disorder; and/or

iv. inhibiting, relieving, ameliorating, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

“EC₅₀,” refers to a dosage, concentration or amount of a particular test compound that elicits a dose-dependent response at 50% of maximal expression of a particular response that is induced, provoked or potentiated by the particular test compound.

“IC₅₀” refers to an amount, concentration or dosage of a particular test compound that achieves a 50% inhibition of a maximal response in an assay that measures such response.

As used herein, the term “E1,” “E1 enzyme,” or “E1 activating enzyme” refers to any one of a family of related ATP-dependent activating enzymes involved in activating or promoting ubiquitin or ubiquitin-like (collectively “ubl”) conjugation to target molecules. E1 activating enzymes function through an adenylation/thioester intermediate formation to transfer the appropriate ubl to the respective E2 conjugating enzyme through a transthiolation reaction. The resulting activated ubl-E2 promotes ultimate conjugation of the ubl to a target protein. A variety of cellular proteins that play a role in cell signaling, cell cycle, and protein turnover are substrates for ubl conjugation which is regulated through E1 activating enzymes (e.g., NAE, UAE, SAE). Unless otherwise indicated by context, the term “E1 enzyme” is meant to refer to any E1 activating enzyme protein, including, without limitation, nedd8 activating enzyme (NAE (APPBPI/Uba3)), ubiquitin activating enzyme (UAE (Uba1)), sumo activating enzyme (SAE (Aosl/Uba2)), or ISG15 activating enzyme (Ube1L), preferably human NAE, SAE or UAE, and more preferably NAE.

The term “E1 enzyme inhibitor” or “inhibitor of E1 enzyme” is used to signify a compound having a structure as defined herein, which is capable of interacting with an E1 enzyme and inhibiting its enzymatic activity. Inhibiting E1 enzymatic activity means reducing the ability of an E1 enzyme to activate ubiquitin like (ubl) conjugation to a substrate peptide or protein (e.g., ubiquitination, neddylation, sumoylation). In various embodiments, such reduction of E1 enzyme activity is at least about 50%, at least about 75%, at least about 90%, at least about 95%, or at least about 99%. In various embodiments, the concentration of E1 enzyme inhibitor required to reduce an E1 enzymatic activity is less than about 1 μM, less than about 500 nM, less than about 100 nM, less than about 50 nM, or less than about 10 nM.

### Methods of Preparation

The compounds of the present disclosure may be prepared by use of known chemical reactions and procedures. Representative methods for synthesizing compounds of the disclosure are presented below. It is understood that the nature of the substituents required for the desired target compound often determines the preferred method of synthesis. All variable groups of these methods are as described in the generic description if they are not specifically defined below.

### General Procedure

Representative synthetic procedures for the preparation of compounds of the disclosure are outlined below in following schemes. Unless otherwise indicated, R₁ₐ, R_{1b}, R₂, R₃ₐ, R_{3b}, R₄ₐ, R_{4b}, R₅-R₁₂, X, and Y, and carry the definitions given in connection with formula (I).

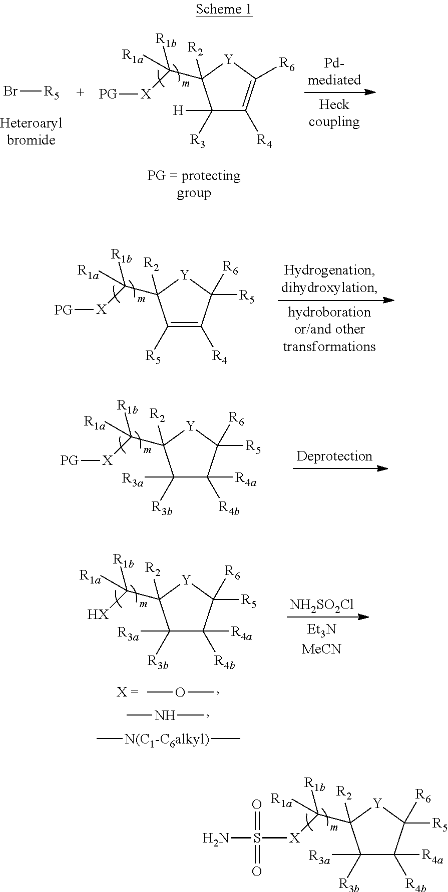

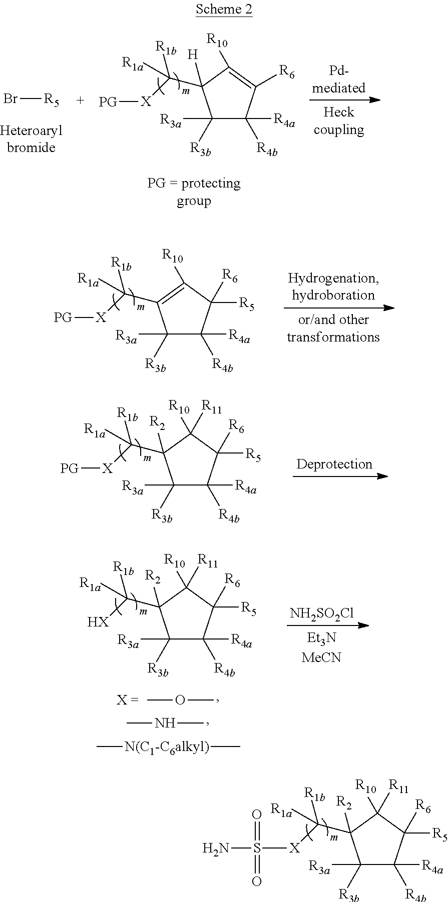

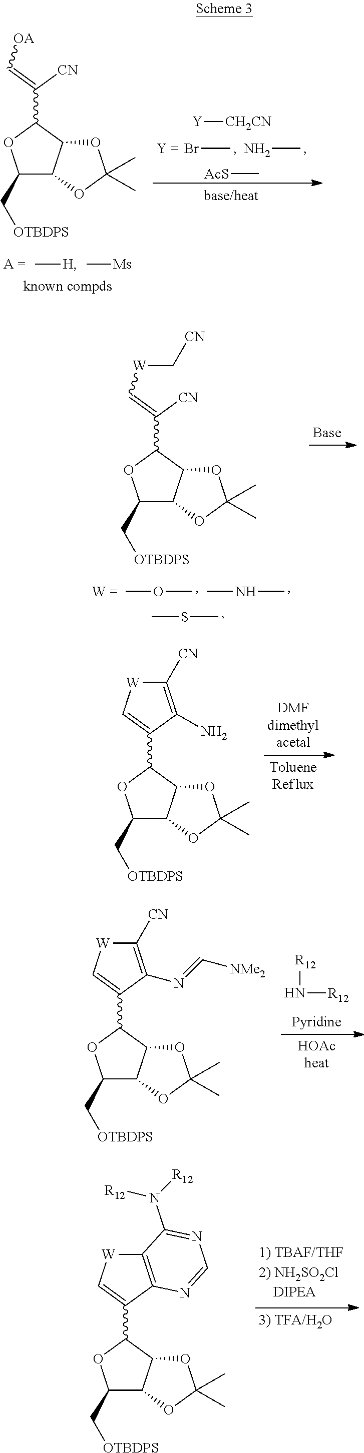

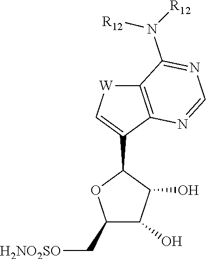

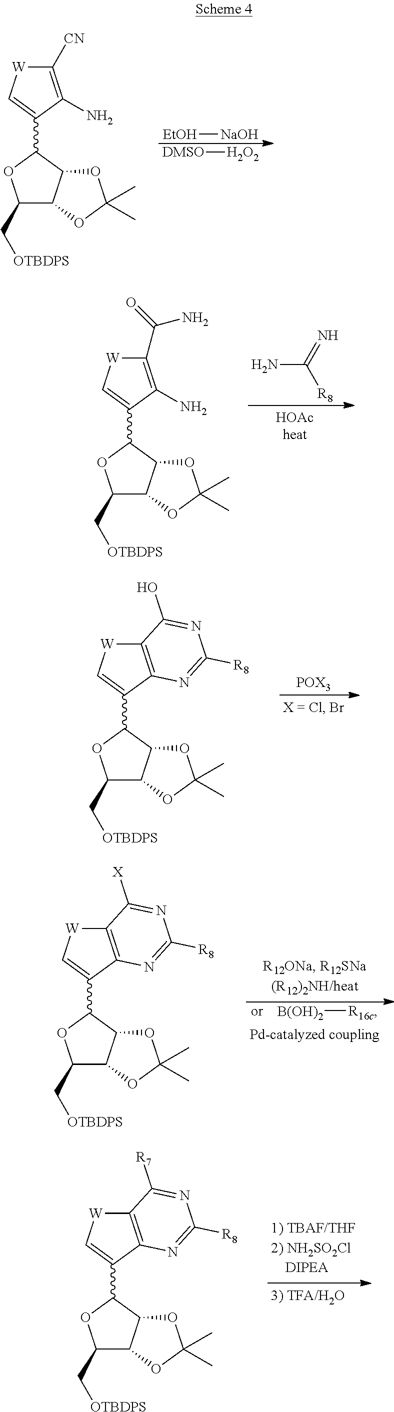

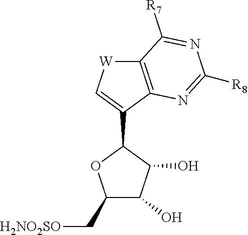

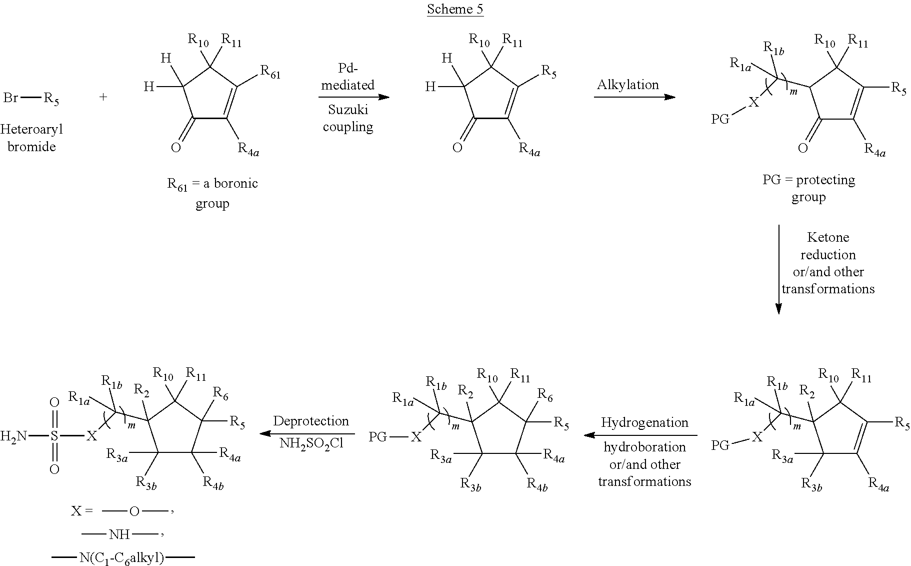

Those having skill in the art will recognize that the starting materials and reaction conditions may be varied, the sequence of the reactions altered, and additional steps employed to produce compounds encompassed by the present disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Starting materials can be obtained from commercial sources or prepared by well-established literature methods known to those of ordinary skill in the art. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/l, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The disclosures of all articles and references mentioned in this application, including patents, are incorporated herein by reference in their entirety.

### EXAMPLES

The preparation of the compounds of the disclosure is illustrated further by the following examples, which are not to be construed as limiting the disclosure in scope or spirit to the specific procedures and compounds described in them. In all cases, unless otherwise specified, the column chromatography is performed using a silica gel solid phase.

Compound identity and purity confirmations are performed by LC/UV/MS using a Waters Micromass ZQ™ Detector and Waters 2695 Separations Module and Waters 2487 Dual λ Absorbance Detector (Waters Corporation, Milford, Mass.). The diode array detector wavelength is 254 nm, and the MS is operated in positive electrospray ionization mode. The samples are maintained at room temperature in the autosampler, and an aliquot (5 μL) is injected onto an Ascentis Express C18 column, 30 mm×3 mm, 2.7 μm (Supelco Analytical, Bellefonte, Pa.) maintained at 40° C. The samples are eluted at a flow rate of 1 mL/min with a mobile phase system composed of solvent A (water containing 0.1% formic acid) and B (acetonitrile containing 0.1% formic acid) with an isocratic gradient 90% A for 0.3 min, then with a linear gradient 10% B to 90% B in 3.6 min, and then isocratic for 0.4 min with 90% B. The column was equilibrated back to the initial conditions for 0.4 min before the next run. In a few instances which are indicated in the examples, a long method is used utilizing 10-minute as total run time. Compound polarized mass and retention time (t_{R}), relative UV absorption area are used to assess purity and identity. Further, NMR spectra is utilized to characterize key intermediates and compounds. Optionally, compound R_{f} values on silica TLC plates are measured.

### Example 1

### Preparation of 7-Bromo-4-chloro-pyrrolo[2,1-f][1,2,4]triazine

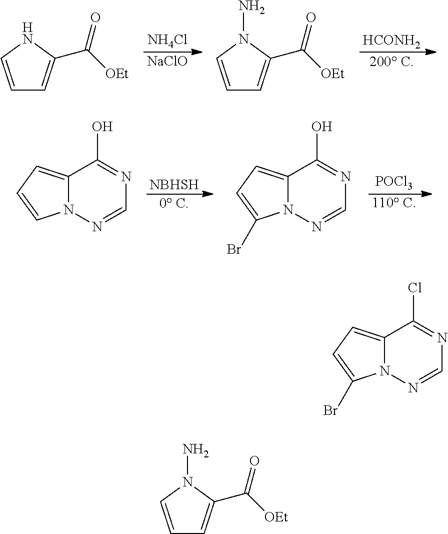

### 1-amino-1H-pyrrole-2-carboxylate

To a mixture of NH₄Cl (41.6 g), Aliquat-336 (1.4 g), 28% aqueous solution of NaOH (360 mL), and 28% aqueous solution of NH₄OH (118 mL) is added at 0° C. a solution of ethyl 1H-pyrrole-2-carboxylate (13.9 g) in methyl tert-butyl ether (372 mL) in one portion. Then a solution of 10% aqueous NaOCl (725 mL) is added slowly at 0° C. over 30 min. The resulting reaction mixture is stirred at room temperature for 4 hours. The organic layer is separated, washed with saturated aqueous Na₂S₂O₃ (150 mL), dried over Na₂SO₄, filtered, and concentrated to give the product ethyl 1-amino-1H-pyrrole-2-carboxylate (11.2 g, 73% yield) as a brown oil.

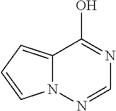

### Pyrrolo[1,2-f][1,2,4]triazin-4-ol

A solution of ethyl 1-amino-1H-pyrrole-2-carboxylate (11.2 g) in formamide (20 mL) is heated under microwave at 200° C. for 1 hour, then poured into saturated aqueous NH₄Cl (300 mL) and the mixture is extracted with ethyl acetate (3×100 mL). The combined organic layer is dried over Na₂SO₄, filtered, concentrated, and purified by silica chromatography eluted by 0-60% of ethyl acetate in hexane to give pyrrolo[1,2-f][1,2,4]triazin-4-ol (5.6 g, 52% yield) as a yellow solid. LCMS (m/z) M+H=136.1; t_{R}=0.62 min.

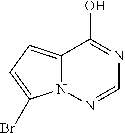

### 7-bromopyrrolo[1,2-f][1,2,4]triazin-4-ol

A stirred solution of pyrrolo[2,1-f][1,2,4]triazin-4-ol (3.7 g) in dichloromethane (60 mL) at 0° C. is added trifluoroacetic acid (30 mL), then N-Bromosuccinimide (3.9 g) in portions over 15 min. The reaction mixture is stirred at 0° C. for 1 additional hour. Then dichloromethane solvent is evaporated. The residue is poured into saturated aq. NaHCO₃ solution (500 mL), stirred for 30 min, filtered to give a solid. The crude product is washed with cold water and hexane, and dried in vacuum to give 7-bromopyrrolo[1,2-f][1,2,4]triazin-4-ol (4.4 g, 75% yield) as a yellow solid. LCMS (m/z) M+H=214.1 and 216.1; t_{R}=2.23 min.

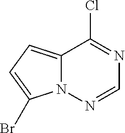

### 7-bromo-4-chloro-pyrrolo[2,1-f][1,2,4]triazine

A solution of 7-bromopyrrolo[2,1-f][1,2,4]triazin-4-ol (4.4 g) in POCl₃ is stirred at 110° C. for 2 hr, cooled to room temperature and poured into ice (500 mL). The mixture is stirred and warmed to room temperature, filtered to give a solid. The crude product is washed with cold water and hexane, and dried in vacuum to give 7-bromo-4-chloro-pyrrolo[2,1-f][1,2,4]triazine (4 g, 84% yield) as a brown solid. LCMS (m/z) M+H=232.0, 234.1, 236.0; t_{R}=4.52 min.

### Example 2

### Preparation of 7-bromo-4-methylsulfanyl-pyrrolo[2,1-f][1,2,4]triazine

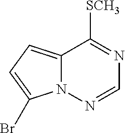

A 40 mL vial is charged with THF (28 mL), 7-bromo-4-chloro-pyrrolo[2,1-f][1,2,4]triazine (4.103 g, 0.017 mol) and methylsulfanylsodium (1.84 g, 0.026 mol). This is stirred for 16 hours at room temperature. The mixture is quenched with saturated aqueous NH₄Cl and extracted twice with EtOAc. The combined organic layers are dried over Na₂SO₄, filtered, concentrated, and purified by silica chromatography eluted by 0-60% ethyl acetate in hexane to give 7-bromo-4-methylsulfanyl-pyrrolo[2,1-f][1,2,4]triazine (3.56 g, 83% yield) as a tan solid. LCMS (m/z) M+H=244.1 and 246.1; t_{R}=3.25 min.

### Example 3

### Preparation of [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methanol

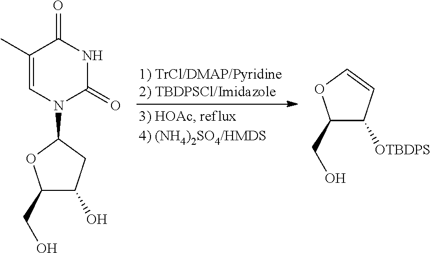

((2R,3S)-3-(tert-butyldiphenylsilyloxy)-2,3-dihydrofuran-2-yl)methanol is prepared in 1 to 10 g scale from thymidine according to a literature procedure (Cameron, M. A.; Cush, S. B.; Hammer, R. P. “Facile Preparation of Protected Furanoid Glycals from Thymidine” J. Org. Chem., 1997, 62(26): 9065-9069).

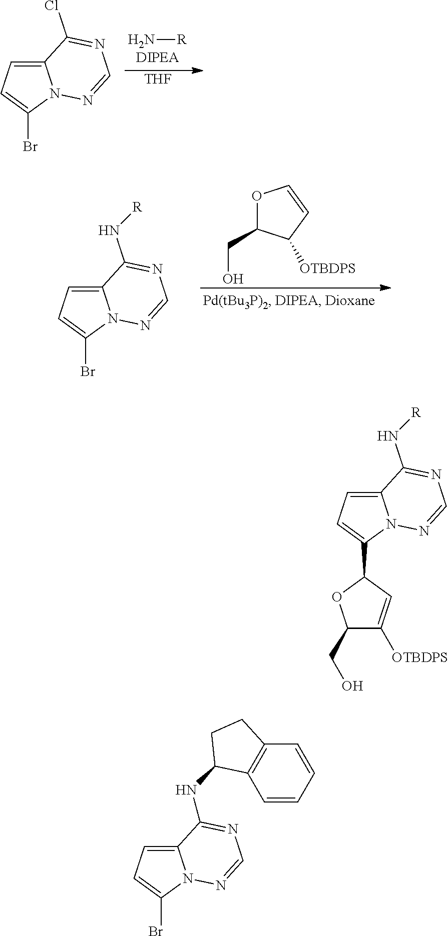

### 7-bromo-N-[(1S)-indan-1-yl]pyrrolo[2,1-f][1,2,4]triazin-4-amine

An 5 mL microwave vial is charged with 7-bromo-4-chloro-pyrrolo[2,1-f][1,2,4]triazine (0.302 g), (1S)-indan-1-amine (0.2078 g), N,N-Diisopropylethylamine (0.543 mL), and THF (3.16 mL). The reaction mixture is heated to 100° C. for 30 min in microwave. The reaction mixture is concentrated onto silica and purified by silica chromatography eluted by 0-100% ethyl acetate in hexane to give 7-bromo-N-[(1S)-indan-1-yl]pyrrolo[2,1-f][1,2,4]triazin-4-amine (0.340 g, 79% yield) as a tan solid. LCMS (m/z) M+H=329.2 and 331.3; t_{R}=2.8 min.

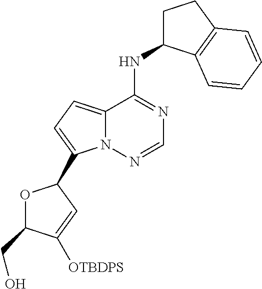

### [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methanol

A 20 mL vial is charged with Dioxane (3 mL) and N,N-Diisopropylethylamine (0.35 mL), 7-bromo-N-[(1S)-indan-1-yl]pyrrolo[2,1-f][1,2,4]triazin-4-amine (0.330 g), [(2R,3S)-3-(tert-butyl(diphenyl)silyl)oxy-2,3-dihydrofuran-2-yl]methanol (0.5318 g), and Pd(tBu₃P)₂ (0.051 g). The reaction vessel is purged with N₂ and heated at 60° C. for 12 hours. The reaction mixture is concentrated onto silica and purified by silica chromatography eluted by 0-50% ethyl acetate in hexane to give [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methanol (0.506 g, 84% yield) as a tan solid. LCMS (m/z) M+H=603.5; t_{R}=3.7 min.

### Example 4

### Preparation of [(2R,5R)-5-[4-[[(1S)-indan-1-yl]amino]pyrrolo[2,1-f][1,2,4]triazin-7-yl]-3-oxo-tetrahydrofuran-2-yl]methyl sulfamate (Compound 1)

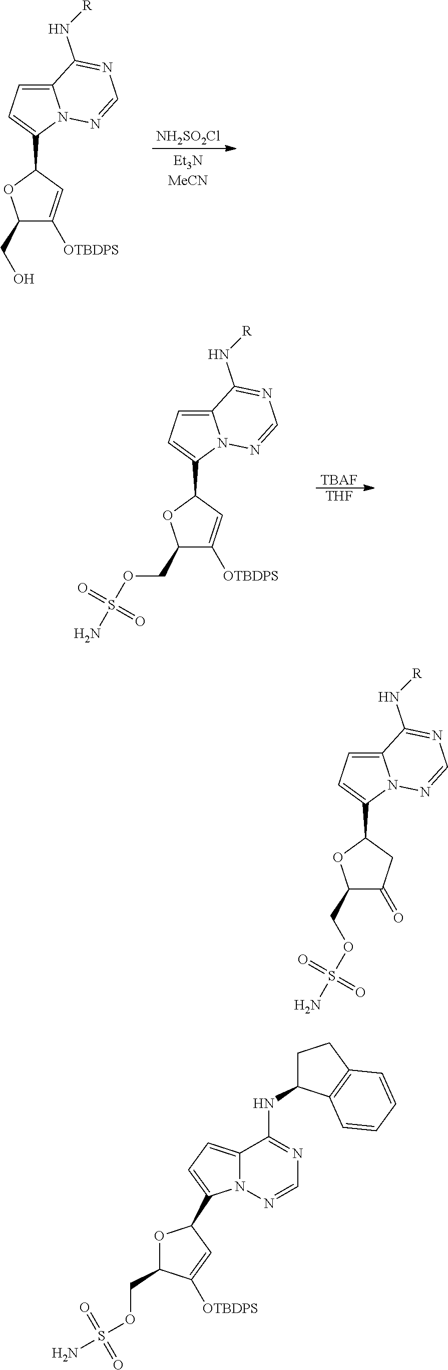

### [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methyl sulfamate

A 20 mL vial is charged with acetonitrile (1 mL), [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methanol (0.120 g) and triethylamine (0.084 mL). The reaction mixture is cooled to 0° C. and added sulfamoyl chloride (0.42 mL, 1.91 M in acetonitrile) and stirred for 10 min then allowed to warm to room temperature for 1 hr. The reaction mixture is concentrated onto celite and purified by silica chromatography eluted by 0-75% ethyl acetate in hexane to give [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methyl sulfamate (0.090 g, 66%) as a tan solid. LCMS (m/z) M+H=682.5; t_{R}=3.5 min.

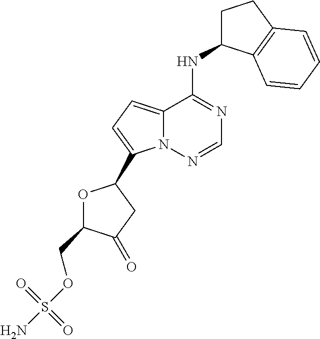

### [(2R,5R)-5-[4-[[(1S)-indan-1-yl]amino]pyrrolo[2,1-f][1,2,4]triazin-7-yl]-3-oxo-tetrahydrofuran-2-yl]methyl sulfamate (Compound 1)

An 8 mL vial is charged with that compound (0.034 g), THF (0.2 mL), cooled to −78° C., added tetrabutylammonium fluoride (0.08 mL, 1 M in THF), and the mixture is stirred for 30 min, monitored by TLC while warming to room temperature. The crude reaction mixture is purified by preparative silica TLC plate eluted by 1:1:0.1 mixture of CH₂Cl₂/EtOAc/MeOH to give [(2R,5R)-5-[4-[[(1S)-indan-1-yl]amino]pyrrolo[2,1-f][1,2,4]triazin-7-yl]-3-oxo-tetrahydrofuran-2-yl]methyl sulfamate (0.02 g, 90%) as a white solid. LCMS (m/z) M+H=444.4; t_{R}=2.1 min.

### Example 5

### Preparation of [(2R,3S,5R)-3-hydroxy-5-[4-[[(1S)-indan-1-yl]amino]pyrrolo[2,1-f][1,2,4]triazin-7-yl]tetrahydrofuran-2-yl]methyl sulfamate (Compound 2)

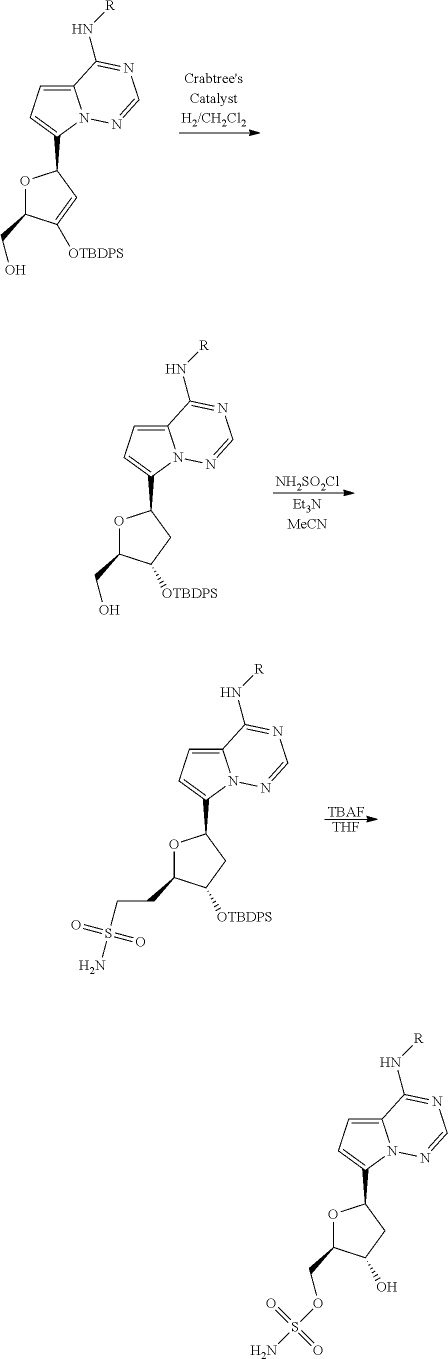

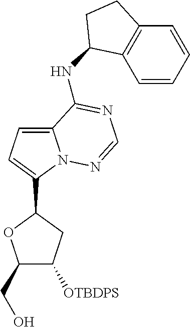

### [(2R,3S,5R)-3-(tert-butyl(diphenyl)silyl)oxy-5-[4-[[(1S)-indan-1-yl]amino]pyrrolo[2,1-f][1,2,4]triazin-7-yl]tetrahydrofuran-2-yl]methanol

A 40 mL vial is charged with [(2R,5R)-3-(tert-butyl(diphenyl)silyl)oxy-5-[4-[[(1S)-indan-1-yl]amino]pyrrolo[2,1-f][1,2,4]triazin-7-yl]-2,5-dihydrofuran-2-yl]methanol (0.506 g) and CH₂Cl₂ (11 mL). The solution is degassed with N₂ for 5 min and added Crabtree's catalyst (68 mg), and solution is again degassed with N₂ for 1 min. The N₂ is removed on high vacuum briefly and backfilled with H₂ via a balloon. Reaction mixture is stirred for 18 hr, added more the catalyst (68 mg), purged with N₂ and backfilled with H₂, stirred for 24 more hr. The mixture is concentrated onto celite and purified by silica chromatography eluted by 0-50% ethyl acetate in hexane to give [(2R,3S,5R)-3-(tert-butyl(diphenyl)silyl)oxy-5-[4-[[(1S)-indan-1-yl]amino]pyrrolo[2,1-f][1,2,4]triazin-7-yl]tetrahydrofuran-2-yl]methanol (0.105 g, 21%) as a tan solid. LCMS (m/z) M+H=605.5; t_{R}=3.4 min.

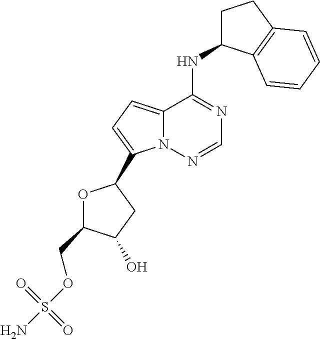

### [(2R,3S,5R)-3-hydroxy-5-[4-[[(1S)-indan-1-yl]amino]pyrrolo[2,1-f][1,2,4]triazin-7-yl]tetrahydrofuran-2-yl]methyl sulfamate (Compound 2)

A 20 mL vial is charged with acetonitrile (1 mL), [(2R,3S,5R)-3-(tert-butyl(diphenyl)silyl)oxy-5-[4-[[(1S)-indan-1-yl]amino]pyrrolo[2,1-f][1,2,4]triazin-7-yl]tetrahydrofuran-2-yl]methanol (0.105 g) and triethylamine (0.084 mL). The mixture is cooled to 0° C., added sulfamoyl chloride (0.42 mL, 1.91 M in MeCN) and stirred for 10 min then allowed to warm to room temperature for 1 hr. The reaction mixture is concentrated onto celite and purified by silica chromatography eluted by 0-75% ethyl acetate in hexane to give [(2R,3S,5R)-3-(tert-butyl(diphenyl)silyl)oxy-5-[4-[[(1 Sy indan-1-yl]amino]pyrrolo[2,1-f][1,2,4]triazin-7-yl]tetrahydrofuran-2-yl]methyl sulfamate (0.020 g, 15%) as a tan solid. LCMS (m/z) M+H=684.5; t_{R}=3.4 min.

An 8 mL vial is charged with THF (0.2 mL) and above compound (0.034 g), and cooled to −78° C. Tetrabutylammonium fluoride (0.08 mL, 1 M in THF) is added and the mixture is stirred for 30 min while warming to room temperature and the reaction is monitored by TLC. The crude reaction mixture is purified by preparative silica TLC plate eluted by 1:1:0.1 mixture of CH₂Cl₂/EtOAc/MeOH to give [(2R,3S,5R)-3-hydroxy-5-[4-[[(1S)-indan-1-yl]amino]pyrrolo[2,1-f][1,2,4]triazin-7-yl]tetrahydrofuran-2-yl]methyl sulfamate (0.0012 g, 6%) as a white solid. LCMS (m/z) M+H=446.4; t_{R}=2.1 min.

### Example 6

### Preparation of [(2R,3S,4R,5S)-5-(7-amino-1H-pyrazolo[4,3-d]pyrimidin-3-yl)-3,4-dihydroxy-tetrahydrofuran-2-yl]methyl sulfamate (Compound 3)

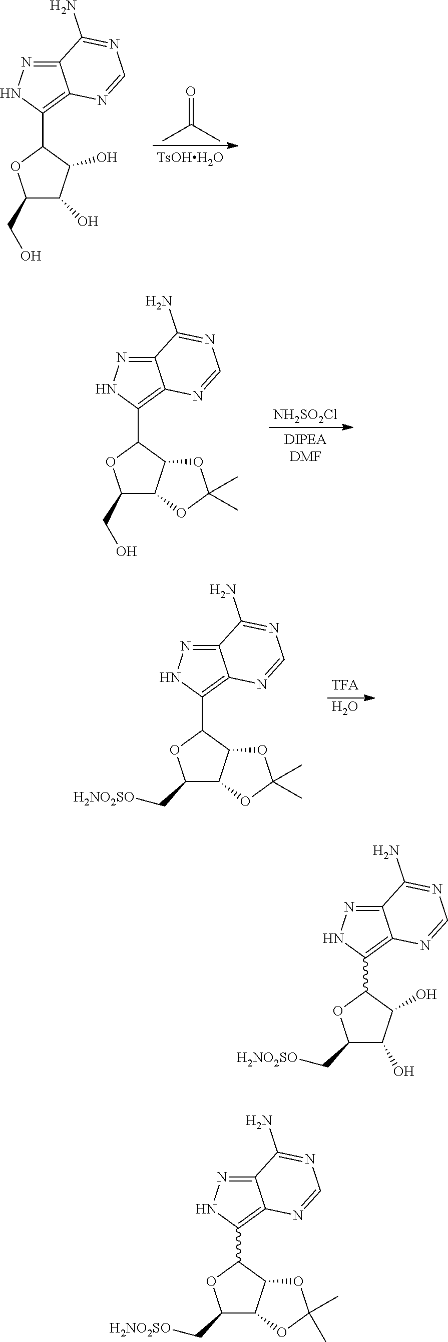

### [(3aR,4R,6S,6aS)-6-(7-amino-1H-pyrazolo[4,3-d]pyrimidin-3-yl)-2,2-dimethyl-3a,4,6,6a-tetrahydrofuro[3,4-d][1,3]dioxol-4-yl]methyl sulfamate

(2S,3R,4S,5R)-2-(7-amino-2H-pyrazolo[4,3-c]pyrimidin-3-yl)-5-(hydroxymethyl)tetrahydrofuran-3,4-diol (10 mg, 0.035 mmol) is dissolved in acetone (0.16 mL). To the solution is added 2,2-dimethoxypropane (8 mg, 0.077 mmol) and p-toluenesulfonic acid (5 mg, 0.026 mmol). The resulting mixture is stirred at room temperature overnight. The reaction mixture is concentrated to give the crude [(3aS,4S,6R,6aR)-4-(7-amino-1H-pyrazolo[4,3-d]pyrimidin-3-yl)-2,2-dimethyl-3a,4,6,6a-tetrahydrofuro[3,4-d][1,3]dioxol-6-yl]methanol, which is used directly in the next step. LCMS (m/z) M+H=308.2; t_{R}=0.54 min.

That crude product is dissolved in DMF (0.5 mL). To the mixture is added N,N-Diisopropylethylamine (0.1 mL). The reaction mixture is cooled to 0° C., and a solution of NH₂SO₂Cl (1.1 eq., 2 M in acetonitrile) is added dropwise. The resulting solution is stirred at 0° C. for 1 hour. The solvents are removed in vacuum, and the residue is purified by preparative silica TLC to give [(3aR,4R,6S,6aS)-6-(7-amino-1H-pyrazolo[4,3-d]pyrimidin-3-yl)-2,2-dimethyl-3a,4,6,6a-tetrahydrofuro[3,4-d][1,3]dioxol-4-yl]methyl sulfamate (4.9 mg, 36% for two steps). LCMS (m/z) M+H=387.2; t_{R}=0.34 min.

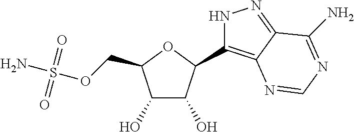

### [(2R,3S,4R,5S)-5-(7-amino-1H-pyrazolo[4,3-d]pyrimidin-3-yl)-3,4-dihydroxy-tetrahydrofuran-2-yl]methyl sulfamate (Compound 3)

[(3aR,4R,6S,6aS)-6-(7-amino-1H-pyrazolo[4,3-d]pyrimidin-3-yl)-2,2-dimethyl-3a,4,6,6a-tetrahydrofuro[3,4-d][1,3]dioxol-4-yl]methyl sulfamate is dissolved in a mixture of trifluoroacetic acid (0.9 mL) and water (0.1 mL). The reaction mixture is stirred at room temperature for 1 hour until TLC showed the deprotection reaction is complete. The solvents are evaporated and the residue is further dried in vacuum to give [(2R,3S,4R,5S)-5-(7-amino-1H-pyrazolo[4,3-d]pyrimidin-3-yl)-3,4-dihydroxy-tetrahydrofuran-2-yl]methyl sulfamate (3.9 mg, 89%) as a solid. LCMS (m/z) M+H=347.2; t_{R}=0.28 min.

### Example 7

### Preparation of [(2R,3S,4R,5S)-5-(4-aminothieno[3,2-d]pyrimidin-7-yl)-3,4-dihydroxy-tetrahydrofuran-2-yl]methyl sulfamate (Compound 4)

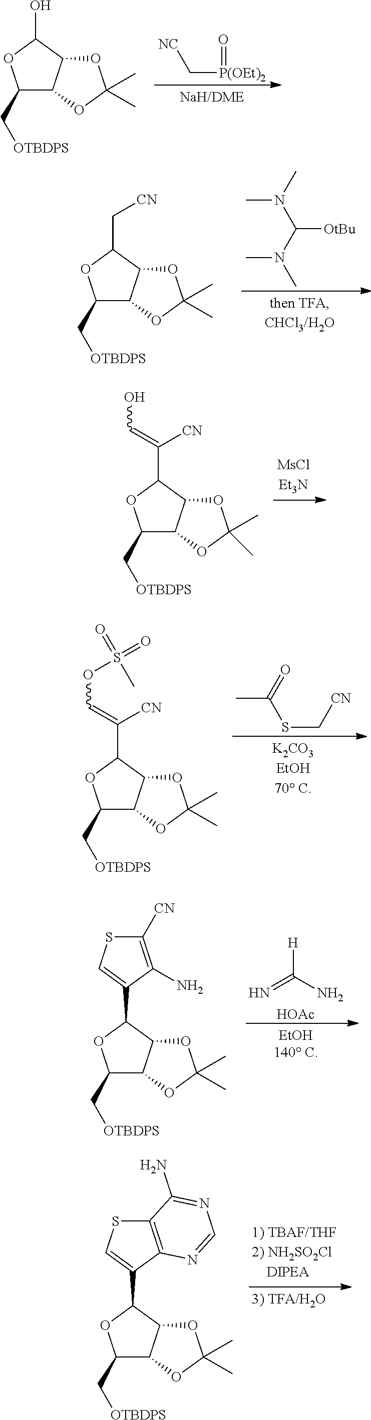

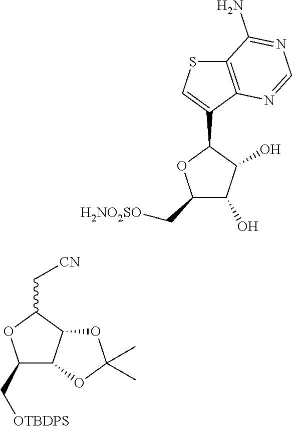

### 2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]acetonitrile

A suspension of NaH (85 mg, 2.1 mmol, 60% in mineral oil) in 1,2-dimethoxyethane (DME, 6.5 mL) is cooled to 0° C. and added diethyl cyanomethylphosphonate (350 mg, 2.10 mmol) dropwise. After stirring for 10 min at 0° C., to the mixture is added a solution of (3aR,6R,6aR)-6-((tert-butyldiphenylsilyloxy)methyl)-2,2-dimethyltetrahydrofuro[3,4-d][1,3]dioxol-4-ol (472 mg, 1.10 mmol) in DME (2.5 mL). The resulting mixture is stirred at room temperature for 2 hrs, added tert-butyl methyl ether (TBME, 50 mL) and water (25 mL), and the organic layer is separated. The aqueous phase is further extracted with TBME and the combined organic solutions are washed with brine (25 mL×2), dried over Na₂SO₄, filtered, the solution is concentrated. The residue is purified by silica gel column chromatography eluted by 1:10 EtOAc/hexane to give 2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]acetonitrile (330 mg, 66% yield) as a colorless oil. LCMS (m/z) M+H=452.2; t_{R}=4.10 min with the long method; ¹H NMR (500 MHz, DMSO-d₆) δ 7.68-7.63 (m, 4H), 7.50-7.42 (m, 6H), 4.81 (d, J=6.1 Hz, 0.5H), 4.72-4.68 (m, 1H), 4.50-4.46 (m, 0.5H), 4.32-4.26 (m, 0.5H), 4.12-4.08 (m, 1H), 4.03 (q, J=4.1 Hz, 0.5H), 3.76-3.64 (m, 2H), 2.93 (dd, J=16.8, 5.2 Hz, 0.5H), 2.85 (dd, J=16.8, 5.1 Hz, 0.5H), 2.78 (dd, J=16.8, 7.3 Hz, 0.5H), 2.69 (dd, J=16.8, 7.8 Hz, 0.5H), 1.46 (s, 1.5H), 1.42 (s, 1.5H), 1.28 (s, 3H), 1.00 (s, 9H).

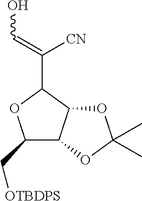

### (E/Z)-2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]-3-hydroxy-prop-2-enenitrile

A solution of 2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]acetonitrile (330 mg, 0.73 mmol) in DMF (4 mL) is added tBuOCH(NMe₂)₂ (990 mg, 5.69 mmol) and the resulting mixture is stirred at room temperature overnight. The reaction mixture is diluted with toluene (80 mL), washed with water (30 mL×2), brine (30 mL), and dried over Na₂SO₄. After filtration, the solution is concentrated to give 2-((3aS,6R,6aR)-6-((tert-butyldiphenylsilyloxy)methyl)-2,2-dimethyltetrahydrofuro[3,4-d][1,3]dioxol-4-yl)-3-(dimethylamino)acrylonitrile as a yellow oil. LCMS (m/z) M+H=507.6; t_{R}=3.61, 3.76 min; ¹H NMR (500 MHz, DMSO-d₆) δ 7.70-7.60 (m, 4H), 7.50-7.41 (m, 6H), 6.97 (s, 0.5H), 6.60 (s, 0.5H), 4.70 (d, J=6.1 Hz, 0.5H), 4.58 (dd, J=6.7, 3.9 Hz, 0.5H), 4.53 (dd, J=5.7, 4.0 Hz, 0.5H), 4.50-4.46 (m, 0.5H), 4.33 (d, J=4.0 Hz, 0.5H), 4.15 (d, J=5.5 Hz, 0.5H), 4.01 (t, J=5.1 Hz, 0.5H), 3.89-3.85 (m, 0.5H), 3.73 (d, J=5.1 Hz, 1H), 3.68 (dd, J=11.0, 5.3 Hz, 0.5H), 3.63 (dd, J=11.0, 5.3 Hz, 0.5H), 3.04 (s, 3H), 3.02 (s, 3H), 1.46 (s, 1.5H), 1.43 (s, 1.5H), 1.27 (s, 1.5H), 1.25 (s, 1.5H), 1.01 (s, 9H).

This is directly dissolved in chloroform (11 mL) and added a solution of TFA (0.17 mL) in water (8 mL). The reaction mixture is stirred vigorously at room temperature overnight. The organic layer is separated, washed with water (5 mL×2), dried over Na₂SO₄, filtered and the solution is concentrated to give crude (E/Z)-2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]-3-hydroxy-prop-2-enenitrile. LCMS (m/z) M+H=480.2; t_{R}=3.18, 3.32 min.

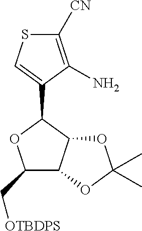

### 4-[(3aS,4S,6R,6aR)-6-[(tert-butyl(diphenyl)silyl)oxymethyl]-2,2-dimethyl-3a,4,6,6a-tetrahydrofuro[3,4-d][1,3]dioxol-4-yl]-3-amino-thiophene-2-carbonitrile

A solution of crude (E/Z)-2-[(3aR,4R,6aS)-4-[(tert-butyl(diphenyl)silyl)oxymethyl]-2,2-dimethyl-3a,4,6,6a-tetrahydrofuro[3,4-d][1,3]dioxol-6-yl]-3-hydroxy-prop-2-enenitrile (355 mg, 0.73 mmol) in dichloromethane (3 mL) is cooled to 0° C. To this is added triethylamine (89 mg, 0.88 mmol) followed by methanesulfonyl chloride (101 mg, 0.88 mmol) in dichloromethane (1.8 mL) over a period of 10 min. The reaction mixture is stirred at 0° C. for 1 h, then diluted with chloroform (15 mL) and washed with water (5 mL×3). The organic layer is separated, dried over Na₂SO₄, filtered and concentrated to give crude [(E/Z)-2-[(3aS,6R,6aR)-6-[(tert-butyl(diphenyl)silyl)oxymethyl]-2,2-dimethyl-3a,4,6,6a-tetrahydrofuro[3,4-d][1,3]dioxol-4-yl]-2-cyano-vinyl]methanesulfonate, which is used as such in the next step. LCMS (m/z) M+H=558.2; t_{R}=3.67 min.

The crude product from last step is dissolved in EtOH (10 mL), added S-(cyanomethyl)ethanethioate (168 mg, 1.46 mmol) and Na₂CO₃ (155 mg, 1.46 mmol). The mixture is refluxed under N₂ for 6 hours until TLC showed the reaction is complete. The mixture is filtered and filtrate is concentrated to dryness. The residue is partitioned between CHCl₃ (15 mL) and water (10 mL). The organic layer is separated, washed with water (10 mL×2), dried over Na₂SO₄, filtered and the solvent is evaporated. The residue is purified by silica gel column chromatography to give 4-[(3aS,4S,6R,6aR)-6-[(tert-butyl(diphenyl)silyl)oxymethyl]-2,2-dimethyl-3a,4,6,6a-tetrahydrofuro[3,4-d][1,3]dioxol-4-yl]-3-amino-thiophene-2-carbonitrile as a colorless oil (146 mg, 37% yield for 4 steps). LCMS (m/z) M+H=535.4; t_{R}=3.57 min.

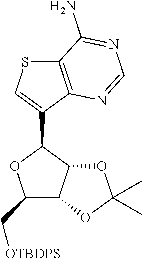

### 7-[(3aS,4S,6R,6aR)-6-[(tert-butyl(diphenyl)silypoxymethyl]-2,2-dimethyl-3a,4,6,6a-tetrahydrofuro[3,4-d][1,3]dioxol-4-yl]thieno[3,2-d]pyrimidin-4-amine

4-[(3aS,4S,6R,6aR)-6-[(tert-butyl(diphenyl)silyl)oxymethyl]-2,2-dimethyl-3a,4,6,6a-tetrahydrofuro[3,4-d][1,3]dioxol-4-yl]-3-amino-thiophene-2-carbonitrile (74 mg, 0.14 mmol), formamidine acetate (230 mg), diethylisopropylamine (358 mg, 2.78 mmol) are dissolved in 1-butanol (3 mL). The mixture is placed in a 35 mL microwave vessel, and heated at 150° C. with microwave for 1.5 hours. The reaction mixture is diluted with EtOAc, and washed with water (10 mL×3). The organic layer is dried over Na₂SO₄, and concentrated to dryness. The residue is purified by column chromatography on silica gel to give recovered starting material (40 mg) and the product 7-[(3aS,4S,6R,6aR)-6-[(tert-butyl(diphenyl)silyl)oxymethyl]-2,2-dimethyl-3a,4,6,6a-tetrahydrofuro[3,4-d][1,3]dioxol-4-yl]thieno[3,2-d]pyrimidin-4-amine (14 mg, 39% based on recovered starting material). LCMS (m/z) M+H=562.3; t_{R}=5.81 min with the long method.

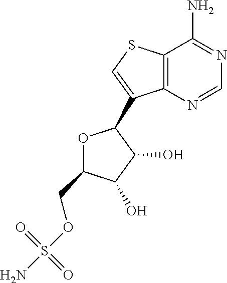

### [(2R,3S,4R,5S)-5-(4-aminothieno[3,2-d]pyrimidin-7-yl)-3,4-dihydroxy-tetrahydrofuran-2-yl]methyl sulfamate (Compound 4)

7-[(3aS,4S,6R,6aR)-6-[(tert-butyl(diphenyl)silyl)oxymethyl]-2,2-dimethyl-3a,4,6,6a-tetrahydrofuro[3,4-d][1,3]dioxol-4-yl]thieno[3,2-d]pyrimidin-4-amine (7.0 mg, 0.012 mmol) in THF (0.5 mL) is added tetrabutylammonium fluoride (0.5 mL, 1 M solution in THF) and the mixture is stirred at room temperature. After LCMS and TLC showed the reaction is complete, the mixture is concentrated to dryness to give crude [(3aS,4S,6R,6aR)-4-(4-aminothieno[3,2-d]pyrimidin-7-yl)-2,2-dimethyl-3a,4,6,6a-tetrahydrofuro[3,4-d][1,3]dioxol-6-yl]methanol. LCMS (m/z) M+H=324.1; t_{R}=0.56 min.

The crude product is dissolved in DMF (0.5 mL), the resulting solution is added N,N-Diisopropylethylamine (0.1 mL) and aminosulfonyl chloride (0.1 mL, 2 M solution in MeCN) at 0° C., and the mixture is stirred at 0° C. for 1 hour. The mixture is concentrated in vacuum to remove the solvents, and the residue is re-dissolved in EtOAc (4 mL) and washed with brine (2 mL). The organic layer is dried over Na₂SO₄, and concentrated to dryness. The residue is purified by preparative TLC to give [(3aS,4S,6R,6aR)-4-(4-aminothieno[3,2-d]pyrimidin-7-yl)-2,2-dimethyl-3a,4,6,6a-tetrahydrofuro[3,4-d][1,3]dioxol-6-yl]methyl sulfamate (4.0 mg, 80% two steps). LCMS (m/z) M+H=403.1; t_{R}=1.07 min.

This compound (4.0 mg, 9.9 mmol) is added a solution of trifluoroacetice acid and water (90% TFA, prepared from 0.45 mL trifluoroacetic acid and 0.05 mL water). The mixture is stirred at room temperature for 30 min, concentrated to dryness, the residue is taken up in EtOAc (5 mL) and washed with water (1 mL). The EtOAc layer is separated, dried over Na₂SO₄, filtered and concentrated to give [(2R,3S,4R,5S)-5-(4-aminothieno[3,2-d]pyrimidin-7-yl)-3,4-dihydroxy-tetrahydrofuran-2-yl]methyl sulfamate (2.2 mg, 61%). LCMS (m/z) M+H=363.1; t_{R}=0.60 min.

### Example 8

### Preparation of ((3aR,4R,6S,6aS)-6-(4-((S)-2,3-dihydro-1H-inden-1-ylamino)thieno[3,2-d]pyrimidin-7-yl)-2,2-dimethyltetrahydrofuro[3,4-d][1,3]-dioxol-4-yl)methanol

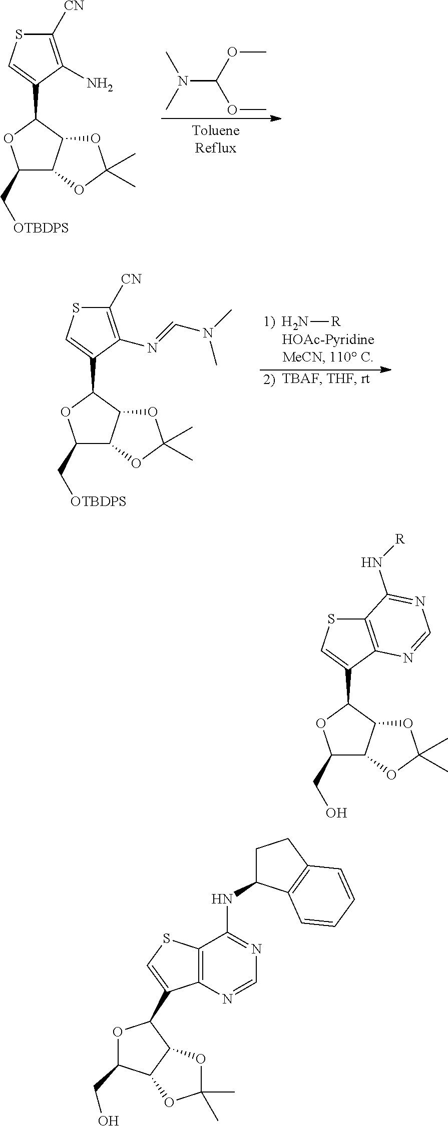

### ((3aR,4R,6S,6aS)-6-(4-((S)-2,3-dihydro-1H-inden-1-ylamino)thieno[3,2-d]pyrimidin-7-yl)-2,2-dimethyltetrahydrofuro[3,4-d][1,3]dioxol-4-yl)methanol

3-Amino-4-((3aS,4S,6R,6aR)-6-((tert-butyldiphenylsilyloxy)methyl)-2,2-dimethyltetrahydrofuro[3,4-d][1,3]dioxol-4-yl)thiophene-2-carbonitrile (107 mg, 0.20 mmol) is dissolved in anhydrous toluene (3 mL). To the solution is added N,N-dimethylformamide dimethyl acetal (36 mg, 0.30 mmol), and the mixture is then heated at 110° C. for 4 hours until TLC and LC-MS showed the reaction is complete. The mixture is concentrated and dried under vacuum to give crude (4-((3aS,4S,6R,6aR)-6-((tert-butyldiphenylsilyloxy)methyl)-2,2-dimethyltetrahydrofuro[3,4-d][1,3]dioxol-4-yl)-2-cyanothiophen-3-yl)-N,N-dimethylformimidamide, which is used directly in the next step. LCMS (m/z) M+H=590.2; t_{R}=3.97 min.

This crude product (39 mg, 0.066 mmol) is placed in a 10 mL microwave reaction vessel with a magnetic stir bar, anhydrous acetonitrile (0.5 mL), (S)-1-aminoindane (27 mg, 0.20 mmol), pyridine (79 mg) and HOAc (60 mg). The resulting mixture is heated with microwave to 110° C. at 30-minute intervals. The reaction is checked by LC-MS after each 30-minute interval until the reaction is complete. The mixture is concentrated, extracted with EtOAc/H₂O, the EtOAc layer is separated, dried, and concentrated. The residue is purified by preparative TLC to give 7-((3aS,4S,6R,6aR)-6-((tert-butyldiphenylsilyloxy)methyl)-2,2-dimethyltetrahydrofuro[3,4-d][1,3]dioxol-4-yl)-N—((S)-2,3-dihydro-1H-inden-1-yl)thieno[3,2-d]pyrimidin-4-amine (22 mg, 49%). LCMS (m/z) M+H=678.3; t_{R}=3.61 min.

This amino compound (22 mg, 0.032 mmol) is dissolved in THF (0.2 mL) and added tetrabutylammonium fluoride (0.1 mL, 1 M in THF). The reaction mixture is stirred at room temperature for 2 hours. The solvent is evaporated and the residue is purified by preparative TLC to give the title product (10 mg, 71%). LCMS (m/z) M+H=440.2; t_{R}=2.12 min.

### Example 9

### Preparation of ((2R,3S,4R,5S)-5-(4-((S)-2,3-dihydro-1H-inden-1-ylamino)thieno[3,2-d]pyrimidin-7-yl)-3,4-dihydroxytetrahydrofuran-2-yl)methyl sulfamate (Compound 5)

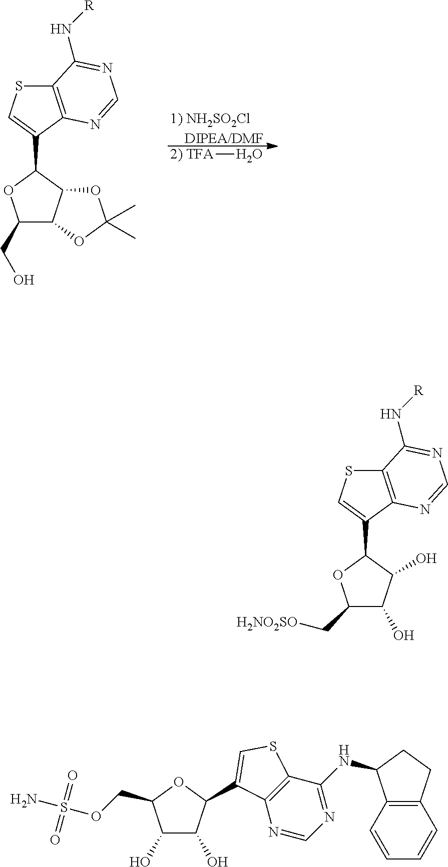

### ((2R,3S,4R,5S)-5-(4-((S)-2,3-dihydro-1H-inden-1-ylamino)thieno[3,2-d]pyrimidin-7-yl)-3,4-dihydroxytetrahydrofuran-2-yl)methyl sulfamate (Compound 5)

((3aR,4R,6S,6aS)-6-(4-((S)-2,3-dihydro-1H-inden-1-ylamino)thieno[3,2-d]pyrimidin-7-yl)-2,2-dimethyltetrahydrofuro[3,4-d][1,3]dioxol-4-yl)methanol (10 mg, 0.023 mmol), N,N-Diisopropylethylamine (150 μL) are dissolved in anhydrous DMF (1 mL). The mixture is cooled to 0° C., slowly added a solution of NH₂SO₂Cl (200 μL, 2 M in acetonitrile). The reaction mixture is stirred at 0° C. for 15 min then graduated warmed to room temperature over 1 hr. The mixture is then worked up by EtOAc—H₂O extraction. The EtOAc layer is separated, concentrated, the residue purified by preparative TLC to give the product ((3aR,4R,6S,6aS)-6-(4-((S)-2,3-dihydro-1H-inden-1-ylamino)thieno[3,2-d]pyrimidin-7-yl)-2,2-dimethyltetrahydrofuro[3,4-d][1,3]dioxol-4-yl)methyl sulfamate (6.2 mg, 52%). LCMS (m/z) M+H=519.3; t_{R}=2.24 min.

This sulfamate product (6.2 mg, 0.012 mmol) is dissolved in a TFA-H₂O solution (1 mL, 7:3), and the mixture is stirred at room temperature for 2 hours until LC-MS showed the deprotection is complete. The reaction mixture is concentrated and purified by preparative TLC to give title compound (4.0 mg, 70%). LCMS (m/z) M+H=479.2; t_{R}=1.89 min.

### Example 10

### Preparation of ((2R,3S,4R,5S)-5-(4-(4-chlorobenzylamino)thieno[3,2-d]pyrimidin-7-yl)-3,4-dihydroxytetrahydrofuran-2-yl)methyl sulfamate (Compound 6)

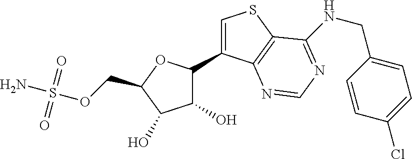

### ((2R,3S,4R,5S)-5-(4-(4-chlorobenzylamino)thieno[3,2-d]pyrimidin-7-yl)-3,4-dihydroxytetrahydrofuran-2-yl)methyl sulfamate (Compound 6)

The title compound is similarly prepared from (4-(3aS,4S,6R,6aR)-6-((tert-butyldiphenylsilyloxy)methyl)-2,2-dimethyltetrahydrofuro[3,4-d][1,3]dioxol-4-yl)-2-cyanothiophen-3-yl)-N,N-dimethylformimidamide (59 mg, 0.10 mmol) in an overall yield of 21% (10 mg). LCMS (m/z) M+H=487.1, 489.1; t_{R}=1.92 min.

### Example 11

### Preparation of (1R,2S)-2-(benzyloxymethyl)cyclopent-3-en-1-ol

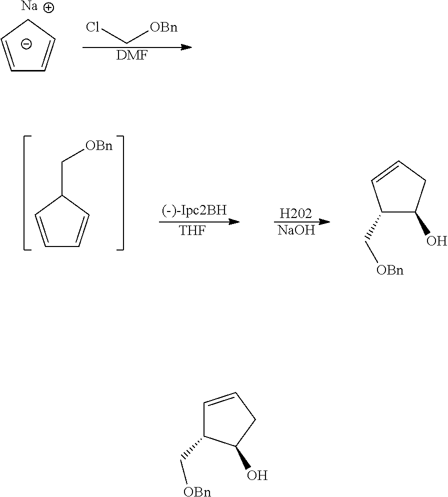

### (1R,2S)-2-(benzyloxymethyl)cyclopent-3-en-1-ol

Borane dimethylsulfide complex (9.48 mL) is added dropwise to a 0° C. solution of (−)-α-pinene (36.64 mL) in THF (205 mL) according to Woll, Matthew G.; Fisk, John D.; LePlae, Paul R.; Gellman, Samuel H. J. Am. Chem. Soc., 2002, 124, 12447-12452. The reaction mixture is stirred at 0° C. for 15 min and then warmed to room temperature to give a fresh suspension of (−)-Ipc₂BH. As the mixture stirred at room temperature, a solution of benzyl chloromethyl ether (15.3 mL, 90% pure) in DMF (232 mL) is cooled to −40° C. and a solution of sodium cyclopentadienylide (0.1 mol) is added dropwise. The resulting mixture is stirred for 30 min and quenched by pouring into hexane (600 mL) and ice water (300 mL). The hexane layer is washed 2 more times with ice water (300 mL), dried over Na₂SO₄ while keeping the solution at 0° C. to prevent isomerization. Organic layer is filtered and concentrated while maintained at 0° C. throughout. The resulting oil is taken up in THF (200 mL) and cooled to −78° C. and the mixture added to the suspension of (−)-Ipc₂BH prepared earlier. The reaction is stirred at −78° C. for 30 min then warmed to 4° C., and stirred for 24 hours. The reaction is then quenched by addition of MeOH (40 mL), aqueous NaOH (40 mL, 3 M) and H₂O₂ (40 mL, 30% in water), and the mixture is stirred for 4 hr. The solvent is removed under reduced pressure, taken up in EtOAc (500 mL) and saturated NaCl aqueous solution (300 mL). The organic layer is dried with Na₂SO₄, filtered, concentrated and purified by silica chromatography eluted by 0-40% ethyl acetate in hexane to give (1R,2S)-2-(benzyloxymethyl)cyclopent-3-en-1-ol (8.2 g, 40% yield) as a clear oil. LCMS (m/z) M+H=205.4; t_{R}=1.96 min.

### Example 12

### Preparation of (1R,4S)-2-(benzyloxymethyl)-4-(4-methylsulfanylpyrrolo[2,1-f][1,2,4]triazin-7-yl)cyclopent-2-en-1-ol

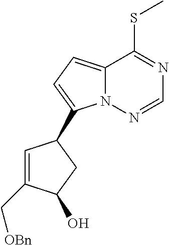

### (1R,4S)-2-(benzyloxymethyl)-4-(4-methylsulfanylpyrrolo[2,1-f][1,2,4]triazin-7-yl)cyclopent-2-en-1-ol

An 8 mL vial is charged with dioxane (3 mL), dicyclohexylmethylamine (0.428 mL), 7-bromo-4-methylsulfanyl-pyrrolo[2,1-f][1,2,4]triazine (0.244 g), (1R,2S)-2-(benzyloxymethyl)cyclopent-3-en-1-ol (0.306 g), and Pd(tBu₃P)₂ (0.051 g). The reaction vessel is purged with N₂ and heated at 60° C. for 12 hours. The mixture is concentrated onto silica and purified by silica chromatography eluted by 0-40% ethyl acetate in hexane to give (1R,4S)-2-(benzyloxymethyl)-4-(4-methylsulfanylpyrrolo[2,1-f][1,2,4]triazin-7-yl)cyclopent-2-en-1-ol (0.256 g, 70% yield) as a tan solid. LCMS (m/z) M+H=368.2; t_{R}=3.5 min.

### Example 13

### Preparation of (1R,4S)-2-(benzyloxymethyl)-4-(4-methylsulfanylpyrrolo[2,1-f][1,2,4]triazin-7-yl)cyclopent-2-en-1-ol

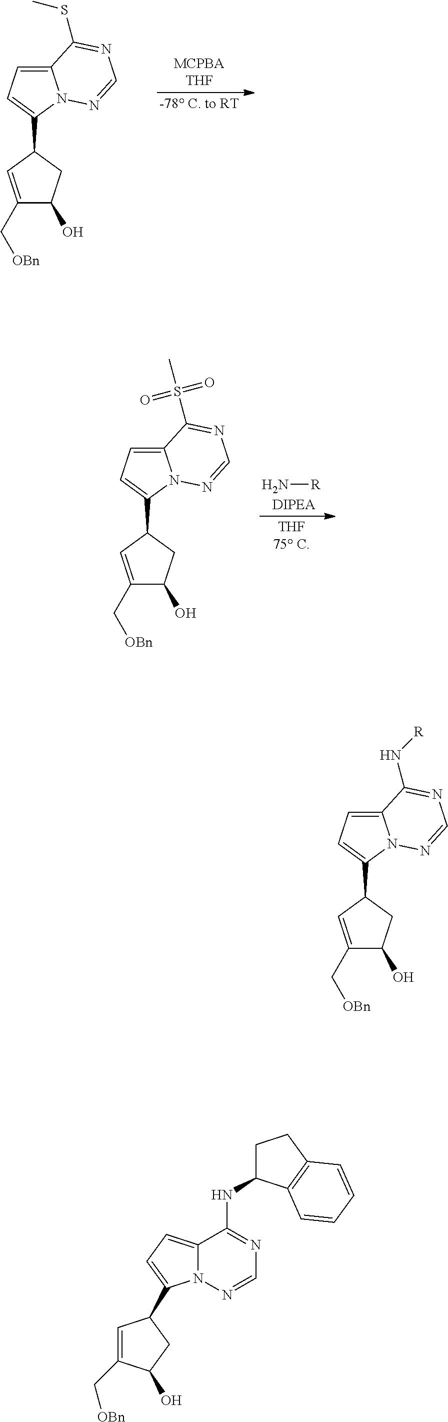

### (1R,4S)-2-(benzyloxymethyl)-4-(4-methylsulfanylpyrrolo[2,1-f][1,2,4]triazin-7-yl)cyclopent-2-en-1-ol

To a solution of (1R,4S)-2-(benzyloxymethyl)-4-(4-methylsulfanylpyrrolo[2,1-f][1,2,4]triazin-7-yl)cyclopent-2-en-1-ol (0.551 g) in THF (4.9 mL) at −78° C. is added m-chloroperbenzoic acid (0.5177 g). The reaction mixture is warmed to room temperature over 30 min and quenched with 1:1 saturated Na₂CO₃ in water and extracted with EtOAc. Organic layers are combined, dried over Na₂SO₄ and concentrated. This crude mixture is used without further purification by adding THF (4.9 mL), N,N-diisopropylethylamine (0.783 mL) and (1S)-indan-1-amine (0.377 mL), then heated at 75° C. for 4 hr. The reaction mixture is quenched with saturated aqueous NH₄Cl and extracted 3 times with EtOAc. The combined organic layers are dried over Na₂SO₄, filtered, concentrated, and purified by silica chromatography eluted by 0-100% ethyl acetate in hexane to give (1R,4S)-2-(benzyloxymethyl)-4-[4-[[(1S)-indan-1-yl]amino]pyrrolo[2,1-f][1,2,4]triazin-7-yl]cyclopent-2-en-1-ol (0.255 g, 38% yield) as a tan solid. LCMS (m/z) M+H=453.5; t_{R}=3.2 min.

### Example 14

### Preparation of [(1R,2S,4R)-2-hydroxy-4-[4-[[(1S)-indan-1-yl]amino]pyrrolo[2,1-f][1,2,4]triazin-7-yl]cyclopentyl]methyl sulfamate (Compound 7) and [(1S,2S,4R)-2-hydroxy-4-[4-[[(1S)-indan-1-yl]amino]pyrrolo[2,1-f][1,2,4]triazin-7-yl]cyclopentyl]methyl sulfamate (Compound 8)

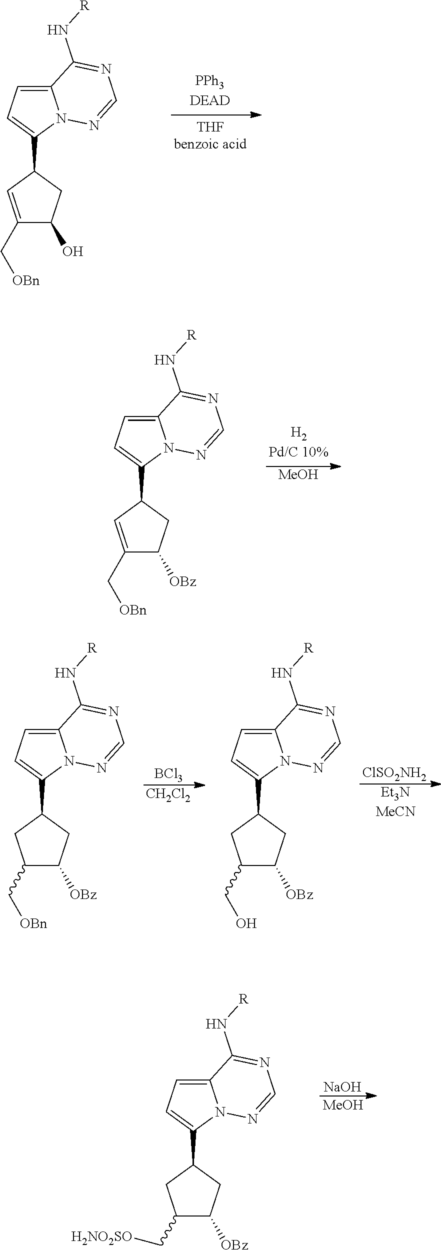

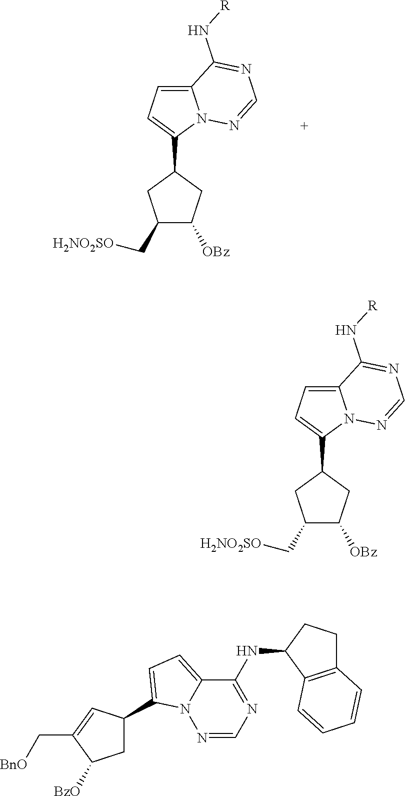

### [(1S,4S)-2-(benzyloxymethyl)-4-[4-[[(1S)-indan-1-yl]amino]pyrrolo[2,1-f][1,2,4]triazin-7-yl]cyclopent-2-en-1-yl]benzoate

A 20 mL vial is charged with THF (2.23 mL), (1R,4S)-2-(benzyloxymethyl)-4-[4-[[(1S)-indan-1-yl]amino]pyrrolo[2,1-f][1,2,4]triazin-7-yl]cyclopent-2-en-1-ol (0.250 g), diethyl azodicarboxylate solution (0.3 mL, 40 wt. % in toluene), triphenylphosphine (0.173 g), and benzoic acid (0.0806 g). The mixture is stirred for 1 hour at room temperature then concentrated onto celite and purified by silica chromatography eluted by 0-50% ethyl acetate in hexane to give [(1S,4S)-2-(benzyloxymethyl)-4-[4-[[(1S)-indan-1-yl]amino]pyrrolo[2,1-f][1,2,4]triazin-7-yl]cyclopent-2-en-1-yl]benzoate (0.177 g, 58% yield) as a white solid. LCMS (m/z) M+H=557.5; t_{R}=3.5 min.

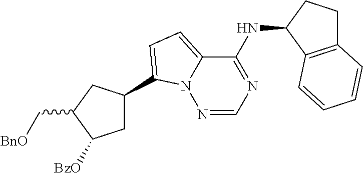

### [(1S,4R)-2-(benzyloxymethyl)-4-[4-[[(1S)-indan-1-yl]amino]pyrrolo[2,1-f][1,2,4]triazin-7-yl]cyclopentyl]benzoate, mixture of two diastereomers

A Parr bottle is charged with MeOH (20 mL), [(1S,4S)-2-(benzyloxymethyl)-4-[4-[[(1S)-indan-1-yl]amino]pyrrolo[2,1-f][1,2,4]triazin-7-yl]cyclopent-2-en-1-yl]benzoate (0.167 g), and 10% Pd(OAc)₂ on carbon (0.800 g). The mxiture is placed under 30 PSI H₂ and shaken for 1 hr. The reaction mixture is filtered and then concentrated onto celite and purified by silica chromatography eluted by 0-75% ethyl acetate in hexane to give a mixture of [(1S,2R,4R)-2-(benzyloxymethyl)-4-[4-[[(1S)-indan-1-yl]amino]pyrrolo[2,1-f][1,2,4]triazin-7-yl]cyclopentyl]benzoate and [(1S,2S,4R)-2-(benzyloxymethyl)-4-[4-[[(1S)-indan-1-yl]amino]pyrrolo[2,1-f][1,2,4]triazin-7-yl]cyclopentyl]benzoate (0.100 g, 60% yield) as a white solid. LCMS (m/z) M+H=559.5; t_{R}=3.4 min; ¹H NMR (500 MHz, DMSO-d₆) δ 8.47 (m, 1H), 7.97 (m, 2H), 7.67 (m, 1H), 7.53 (m, 2H), 7.28 (m, 4H), 7.25 (m, 3H), 7.16 (m, 1H), 6.91 (d, J=5.6 Hz, 1H), 6.44 (d, J=5.2 Hz, 1H), 6.25 (s, 1H), 6.06 (m, 1H), 5.93 (dd, J=7.6, 9.3 Hz, 1H), 4.66 (m, 1H), 4.52 (dd, J=17.3, 12.2 Hz, 2H), 4.21 (s, 2H), 3.03 (m, 2H), 2.90 (m, 2H), 2.57-2.48 (m, 4H), 2.02 (m, 1H), 1.23 (s, 1H).

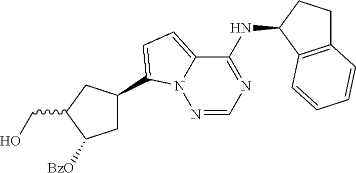

### [(1S,4R)-2-(hydroxymethyl)-4-[4-[[(1S)-indan-1-yl]amino]pyrrolo[2,1-f][1,2,4]triazin-7-yl]cyclopentyl]benzoate, mixture of two diastereomers

A solution of mixture of two diastereomers [(1S,4R)-2-(benzyloxymethyl)-4-[4-[[(1S)-indan-1-yl]amino]pyrrolo[2,1-f][1,2,4]triazin-7-yl]cyclopentyl]benzoate (0.100 g) in CH₂Cl₂ (10 mL) is cooled to −78° C. To this is added BCl₃ (0.72 mL, 1 M in CH₂Cl₂) and the reaction mixture is stirred for 90 min at −40° C. The reaction is quenched at −40° C. by adding MeOH (1.5 mL) and N,N-diisopropylethylamine (1.5 mL), and the mixture is warmed to room temperature then concentrated to dryness. The crude product is taken up in EtOAc, filtered, and purified by silica chromatography eluted by 0-100% ethyl acetate in hexane to give a mixture of [(1S,2R,4R)-2-(hydroxymethyl)-4-[4-[[(1S)-indan-1-yl]amino]pyrrolo[2,1-f][1,2,4]triazin-7-yl]cyclopentyl]benzoate and [(1S,2S,4R)-2-(hydroxymethyl)-4-[4-[[(1S)-indan-1-yl]amino]pyrrolo[2,1-f][1,2,4]triazin-7-yl]cyclopentyl]benzoate (0.24 g, 29% yield) as a white solid. LCMS (m/z) M+H=469.5; t_{R}=2.8 min; ¹H NMR (500 MHz, DMSO-d₆) δ 8.42 (d, J=8.4 Hz, 1H), 8.03 (m, 2H), 7.68 (m, 1H), 7.57 (m, 2H), 7.30 (d, J=7.6 Hz, 1H), 7.24 (m, 2H), 7.19 (m, 1H), 6.94 (d, J=4.5 Hz, 1H), 6.55 (d, J=4.5 Hz, 1H), 5.93 (dd, J=7.7, 8.5 Hz, 1H), 5.57 (m, 1H), 4.50 (t, J=5.05 Hz, 1H), 3.64 (m, 1H), 3.36 (m, 1H), 3.02 (m, 1H), 2.90 (m, 1H), 2.61 (m, 1H), 2.54 (m, 1H), 2.30 (m, 2H), 2.01 (m, 1H), 1.89 (m, 1H), 1.80 (m, 1H), 1.23 (s, 1H).

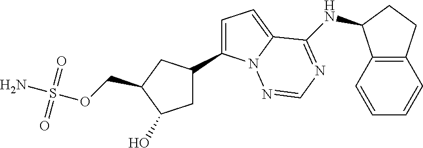

### [(1R,2S,4R)-2-hydroxy-4-[4-[[(1S)-indan-1-yl]amino]pyrrolo[2,1-f][1,2,4]triazin-7-yl]cyclopentyl]methyl sulfamate (Compound 7)

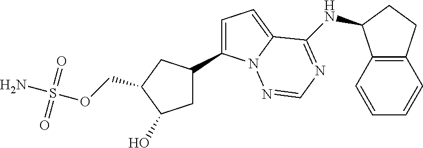

### [(1S,2S,4R)-2-hydroxy-4-[4-[[(1S)-indan-1-yl]amino]pyrrolo[2,1-f][1,2,4]triazin-7-yl]cyclopentyl]methyl sulfamate (Compound 8)

An 8 mL vial is charged with acetonitrile (0.1 mL), mixture of two diastereomers [(1S,4R)-2-(hydroxymethyl)-4-[4-[[(1S)-indan-1-yl]amino]pyrrolo[2,1-f][1,2,4]triazin-7-yl]cyclopentyl]benzoate (0.024 g), and triethylamine (0.015 mL). The reaction mixture is cooled to 0° C., added sulfamoyl chloride (0.037 mL, 1.91 M in MeCN) and stirred for 10 min then allowed to warm to room temperature over 1 hr. An additional amount of sulfamoyl chloride (0.037 mL, 1.91 M in MeCN) is added and the mixture stirred for 1 more hour. The mixture is concentrated, taken up in saturated aqueous NH₄Cl and extracted 3 times with EtOAc. Organic layers are combined, dried over Na₂SO₄, concentrated to give a crude diastereomeric mixture of [(1S,4R)-4-[4-[[(1S)-indan-1-yl]amino]pyrrolo[2,1-f][1,2,4]triazin-7-yl]-2-(sulfamoyloxymethyl)cyclopentyl]benzoate (0.02 g, 100% yield) as a white solid. LCMS (m/z) M+H=548.4; t_{R}=2.8 min.

Without further purification, this crude mixture is charged to an 8 mL vial along with MeOH (0.28 mL) and NaOH (0.021 g). The mixture is stirred at room temperature for 30 min, then concentrated, added acetic acid (0.1 mL) and again concentrated. This is taken up in MeOH and purified by preparative silica TLC plate eluted by 1:1:0.1 CH₂Cl₂/EtOAc/MeOH to give [(1R,2S,4R)-2-hydroxy-4-[4-[[(1S)-indan-1-yl]amino]pyrrolo[2,1-f][1,2,4]triazin-7-yl]cyclopentyl]methyl sulfamate (Compound 7, 0.003 g, 40% yield, t_{R}=2.2 min), and [(1S,2S,4R)-2-hydroxy-4-[4-[[(1S)-indan-1-yl]amino]pyrrolo[2,1-f][1,2,4]triazin-7-yl]cyclopentyl]methyl sulfamate (Compound 8, 0.003 g, 40% yield, t_{R}=2.0 min). Both are white solids and have shown LCMS (m/z) M+H at 444.3.

An alternative diastereoselective synthesis of single [(1S,2S,4R)-2-hydroxy-4-[4-[[(1S)-indan-1-yl]amino]pyrrolo[2,1-f][1,2,4]triazin-7-yl]cyclopentyl]methyl sulfamate (Compound 8) is done by Crabtree's catalyst mediated hydrogenation of [(1S,4S)-2-(benzyloxymethyl)-4-[4-[[(1S)-indan-1-yl]amino]pyrrolo[2,1-f][1,2,4]triazin-7-yl]cyclopent-2-en-1-yl]benzoate, followed by the same subsequent steps as described above.

### Example 15

### Preparation of [(1S,2S,4R)-2-hydroxy-4-[4-[[(1S)-indan-1-yl]amino]thieno[3,2-d]pyrimidin-7-yl]cyclopentyl]methyl sulfamate (Compound 9)

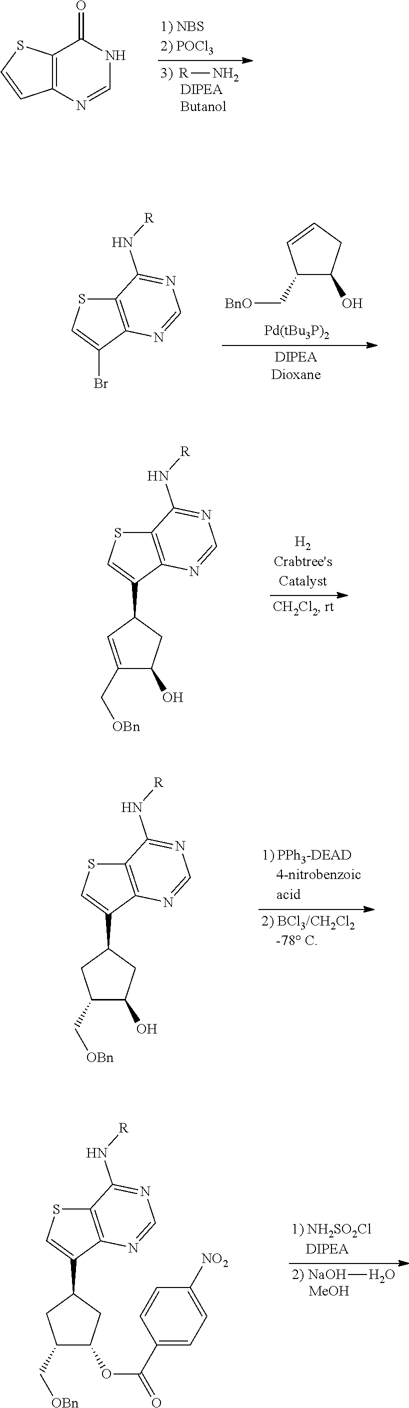

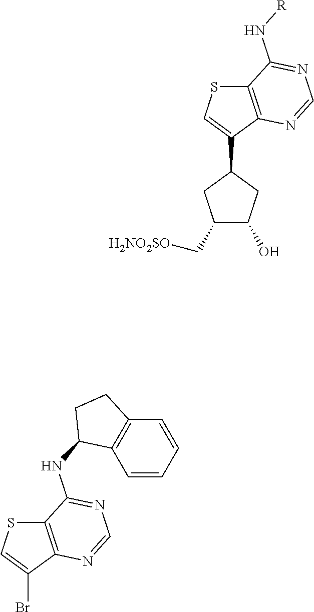

### 7-bromo-N-[(1S)-indan-1-yl]thieno[3,2-d]pyrimidin-4-amine

7-Bromo-4-chloro-thieno[3,2-d]pyrimidine is prepared according to a literature procedure (Thrash, Thomas; Cabell, Larry A.; Lohse, Daniel; Budde, Raymond J. A. US Patent US2006/4002 A1, 2006). A solution of that compound (850 mg, 3.40 mmol), DIPEA (659 mg, 5.10 mmol), and (S)-1-aminoindane (680 mg, 5.10 mmol) in THF (7 mL) is heated at 100° C. under microwave for 3 hours. The solvents are removed under vacuum, and residue is purified by silica gel column chromatography to give 7-bromo-N-[(1S)-indan-1-yl]thieno[3,2-d]pyrimidin-4-amine as a white solid (810 mg, 69%). LCMS: (m/z) M+H=345.9, 347.9; t_{R}=3.10 min.

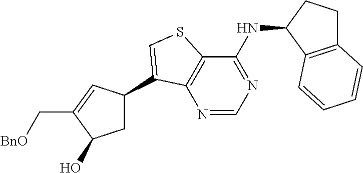

### (1R,4S)-2-(benzyloxymethyl)-4-[4-[[(1S)-indan-1-yl]amino]thieno[3,2-d]pyrimidin-7-yl]cyclopent-2-en-1-ol

In a 50 mL round-bottom flask are charged 7-bromo-N-[(1S)-indan-1-yl]thieno[3,2-d]pyrimidin-4-amine (810 mg, 2.34 mmol), (1R,2S)-2-(benzyloxymethyl)cyclopent-3-enol (956 mg, 4.68 mmol), DIPEA (604 mg, 4.68 mmol), Pd(tBu₃P)₂ (240 mg, 0.47 mmol), 1,4-dioxane (10 mL). The system is degassed and then protected under a N₂ atmosphere. The reaction mixture is heated at 65° C. for 24 hours. After cooling to room temperature, the solvents are evaporated, and the residue is purified by silica gel column chromatography to give (1R,4S)-2-(benzyloxymethyl)-4-[4-[[(1S)-indan-1-yl]amino]thieno[3,2-d]pyrimidin-7-yl]cyclopent-2-en-1-ol (505 mg, 46%) as a solid. LCMS: (m/z) M+H=470.2; t_{R}=3.34 min; ¹H NMR (500 MHz, DMSO-d₆) δ 8.51 (s, 1H), 8.17 (d, J=8.2 Hz, 1H), 7.61 (s, 1H), 7.37-7.33 (m, 4H), 7.32-7.27 (m, 2H), 7.25-7.20 (m, 2H), 7.15 (t, J=7.3 Hz, 1H), 5.97 (q, J=8.0 Hz, 1H), 5.93 (s, 1H), 4.92 (d, J=6.6 Hz, 1H), 4.79-4.74 (m, 1H), 4.54 (d, J=12.0 Hz, 1H), 4.51 (d, J=12.0 Hz, 1H), 4.45-4.40 (m, 1H), 4.18 (d, J=13.4 Hz, 1H), 4.13 (d, J=13.4 Hz, 1H), 3.10-3.00 (m, 2H), 2.91-2.82 (m, 1H), 2.25-2.15 (m, 2H), 2.10-2.00 (m, 1H).

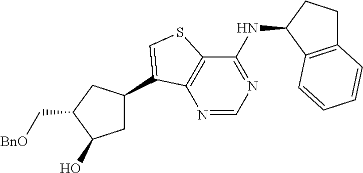

### (1R,2S,4R)-2-(benzyloxymethyl)-4-[4-[[(1S)-indan-1-yl]amino]thieno[3,2-d]pyrimidin-7-yl]cyclopentanol

(1R,4S)-2-(benzyloxymethyl)-4-[4-[[(1S)-indan-1-yl]amino]thieno[3,2-d]pyrimidin-7-yl]cyclopent-2-en-1-ol (35 mg, 0.075 mmol) and Crabtree's catalyst (2 mg) are dissolved in CH₂Cl₂ in a 40 mL scintillation vial equipped with a septum cap. The system is filled with H₂. The reaction mixture is stirred at room temperature for 24 hours. The solution is then concentrated, and the residue is purified by preparative TLC to give (1R,2S,4R)-2-(benzyloxymethyl)-4-[4-[[(1S)-indan-1-yl]amino]thieno[3,2-d]pyrimidin-7-yl]cyclopentanol (32 mg, 91%) as a solid. LCMS: (m/z) M+H=472.2; t_{R}=3.45 min; ¹H NMR (500 MHz, DMSO-d₆) δ 8.51 (s, 1H), 8.16 (d, J=7.6 Hz, 1H), 7.78 (s, 1H), 7.38-7.33 (m, 4H), 7.31-7.26 (m, 2H), 7.25-7.19 (m, 2H), 7.14 (t, J=7.3 Hz, 1H), 5.97 (q, J=7.9 Hz, 1H), 4.50 (s, 2H), 4.26 (q, J=6.3 Hz, 1H), 3.55-3.45 (m, 3H), 3.02 (ddd, J=15.8, 8.7, 2.9 Hz, 1H), 2.91-2.83 (m, 1H), 2.55-2.48 (m, 1H), 2.47-2.40 (m, 1H), 2.30-2.22 (m, 1H), 2.11-1.96 (m, 3H), 1.85-1.75 (m, 1H).

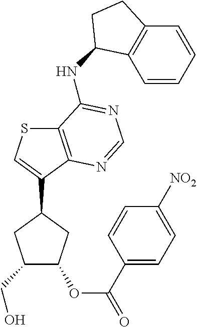

### [(1S,2S,4R)-2-(hydroxymethyl)-4-[4-[[(1S)-indan-1-yl]amino]thieno[3,2-d]pyrimidin-7-yl]cyclopentyl]-4-nitrobenzoate

(1R,2S,4R)-2-(benzyloxymethyl)-4-[4-[[(1S)-indan-1-yl]amino]thieno[3,2-d]pyrimidin-7-yl]cyclopentanol (32 mg, 0.074 mmol), Ph₃P (29 mg, 0.11 mmol), p-nitrobenzoic acid (19 mg, 0.11 mmol) are dissolved in THF (1 mL). The solution is cooled to 0° C., added diethyl azodicarboxylate (0.050 mL, 40% solution in toluene, 0.11 mmol) dropwise. The mixture is stirred at room temperature for 4 hours, concentrated, and the residue is purified by preparative TLC to give [(1S,2S,4R)-2-(benzyloxymethyl)-4-[4-[[(1S)-indan-1-yl]amino]thieno[3,2-d]pyrimidin-7-yl]cyclopentyl]-4-nitrobenzoate (32 mg, 70%) as a solid. LCMS: (m/z) M+H=621.2; t_{R}=8.51 min with the long method.

A solution of this compound (3.0 mg, 4.8 μmol) in dichloromethane (0.5 mL) is cooled to −78° C., added BCl₃ (25 μL in dichloromethane, 25 μmol). The mixture is stirred at −78° C. for 2 hr, concentrated and the residue purified by preparative TLC to give [(1S,2S,4R)-2-(hydroxymethyl)-4-[4-[[(1S)-indan-1-yl]amino]thieno[3,2-d]pyrimidin-7-yl]cyclopentyl]-4-nitrobenzoate (2.0 mg, 78%). LCMS: (m/z) M+H=531.2; t_{R}=6.86 min with the long method.

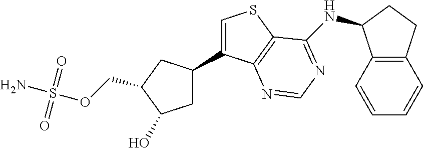

### [(1S,2S,4R)-2-hydroxy-4-[4-[[(1S)-indan-1-yl]amino]thieno[3,2-d]pyrimidin-7-yl]cyclopentyl]methyl sulfamate (Compound 9)

[(1S,2S,4R)-2-(hydroxymethyl)-4-[4-[[(1S)-indan-1-yl]amino]thieno[3,2-d]pyrimidin-7-yl]cyclopentyl]-4-nitrobenzoate is dissolved in DMF (0.5 mL). To this is sequentially added DIPEA (0.1 mL), aminosulfonyl chloride (0.1 mL, 2 M in MeCN) at 0° C., and the solution is stirred for 1 hour. The mixture is concentrated under vacuum to remove the solvents, the residue is re-dissolved in EtOAc (4 mL) and washed with brine (2 mL). The organic layer is dried over Na₂SO₄, filtered, concentrated, and the residue is purified by preparative TLC to give [(1S,2S,4R)-4-[4-[[(1S)-indan-1-yl]amino]thieno[3,2-d]pyrimidin-7-yl]-2-(sulfamoyloxymethyl)cyclopentyl]-4-nitrobenzoate (1.5 mg, 62%). LCMS (m/z) M+H=610.2; t_{R}=7.07 min with the long method. A solution of that product (1.5 mg) in MeOH (0.5 mL) is stirred with an ammonium solution (0.5 mL, 7 M in MeOH) at room temperature for 4 hours until LCMS showed the reaction is complete. The mixture is concentrated, and the residue is purified by preparative TLC to give [(1S,2S,4R)-2-hydroxy-4-[4-[[(1S)-indan-1-yl]amino]thieno[3,2-d]pyrimidin-7-yl]cyclopentyl]methyl sulfamate (1.0 mg, 88%). LCMS (m/z) M+H=461.2; t_{R}=5.54 min with the long method.

### Example 16

### Preparation of ((2R,3S,5R)-5-(4-((S)-2,3-dihydro-1H-inden-1-ylamino)thieno[3,2-d]pyrimidin-7-yl)-3-hydroxytetrahydrofuran-2-yl)methyl sulfamate (Compound 10)

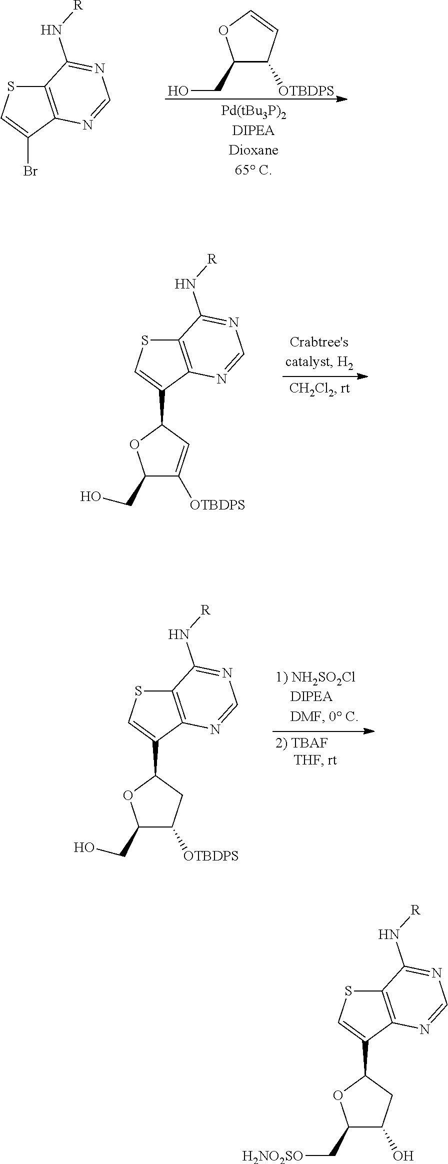

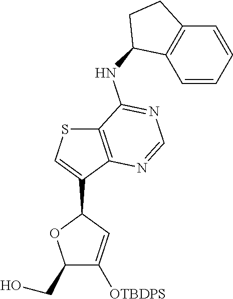

### ((2R,5R)-3-(tert-butyldiphenylsilyloxy)-5-(4-((S)-2,3-dihydro-1H-inden-1-ylamino)thieno[3,2-d]pyrimidin-7-yl)-2,5-dihydrofuran-2-yl)methanol

In a 40 mL scintillation vial equipped with a septum cap are placed (S)-7-bromo-N-(2,3-dihydro-1H-inden-1-yl)thieno[3,2-d]pyrimidin-4-amine (346 mg, 1.0 mmol), ((2R,3S)-3-(tert-butyldiphenylsilyloxy)-2,3-dihydrofuran-2-yl)methanol (710 mg, 2.0 mmol), N,N-Diisopropylethylamine (258 mg, 2.0 mmol), Pd(tBu₃P)₂ (102 mg, 0.20 mmol), and 1,4-dioxane (5 mL). The system is evacuated and then protected under a N₂ atmosphere. The reaction mixture is heated at 65° C. for 12 hours. After cooling to room temperature, the solvents are evaporated and the residue purified by silica gel column chromatography to yield ((2R,5R)-3-(tert-butyldiphenylsilyloxy)-5-(4-((S)-2,3-dihydro-1H-inden-1-ylamino)thieno[3,2-d]pyrimidin-7-yl)-2,5-dihydrofuran-2-yl)methanol (273 mg, 44%). LCMS: (m/z) M+H=620.3; t_{R}=2.92 min.

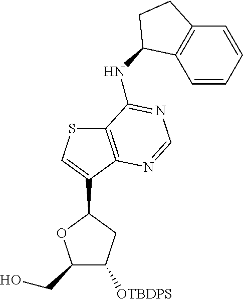

### ((2R,3S,5R)-3-(tert-butyldiphenylsilyloxy)-5-(4-((S)-2,3-dihydro-1H-inden-1-ylamino)thieno[3,2-c]pyrimidin-7-yl)tetrahydrofuran-2-yl)methanol

((2R,5R)-3-(tert-butyldiphenylsilyloxy)-5-(4-((S)-2,3-dihydro-1H-inden-1-ylamino)thieno[3,2-d]pyrimidin-7-yl)-2,5-dihydrofuran-2-yl)methanol (50 mg, 0.081 mmol) and Crabtree's catalyst (16 mg, 0.02 mmol) are dissolved in CH₂Cl₂ (2 mL) in a 40 mL scintillation vial equipped with a septum cap. The system is filled with H₂ and the mixture is stirred at room temperature for 24 hours. The mixture is then concentrated and the residue purified by preparative TLC to give the recovered starting material (22 mg) and ((2R,3S,5R)-3-(tert-butyldiphenylsilyloxy)-5-(4-((S)-2,3-dihydro-1H-inden-1-ylamino)thieno[3,2-d]pyrimidin-7-yl)tetrahydrofuran-2-yl)methanol (24 mg, 86% based on recovered starting material). LCMS: (m/z) M+H=622.3; t_{R}=3.21 min.

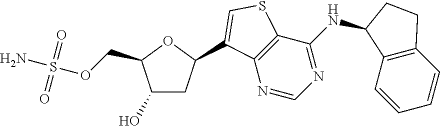

### ((2R,3S,5R)-5-(4-((S)-2,3-dihydro-1H-inden-1-ylamino)thieno[3,2-d]pyrimidin-7-yl)-3-hydroxytetrahydrofuran-2-yl)methyl sulfamate (Compound 10)

((2R,3S,5R)-3-(tert-butyldiphenylsilyloxy)-5-(4-((S)-2,3-dihydro-1H-inden-1-ylamino)thieno[3,2-d]pyrimidin-7-yl)tetrahydrofuran-2-yl)methanol (5.0 mg, 0.008 mmol) is dissolved in DMF (0.5 mL). To the solution are sequentially added N,N-Diisopropylethylamine (0.1 mL) and 2 M aminosulfonyl chloride solution in acetonitrile (0.1 mL) at 0° C. The solution is stirred at 0° C. for 1 hour. The mixture is concentrated in vacuum to remove the solvents, and the residue is re-dissolved in EtOAc (4 mL) and washed with brine (2 mL×2). The organic layer is separated, dried over Na₂SO₄, filtered and concentrated to dryness to give the crude sulfamate product. LCMS (m/z) M+H=701.3; t_{R}=3.36 min. The crude sulfamate from last step is dissolved in THF (0.2 mL) along with tetrabutylammonium fluoride (0.1 mL, 1 M solution in THF). The reaction mixture is stirred at room temperature for 2 hours. The solvent is evaporated and the residue is purified by preparative TLC to give the title compound (1.3 mg, 35% for 2 steps). LCMS (m/z) M+H=463.2; t_{R}=1.89 min.

### Example 17

### Preparation of ((2R,3S,4R,5S)-5-(4-aminofuro[3,2-d]pyrimidin-7-yl)-3,4-dihydroxytetrahydrofuran-2-yl)methyl sulfamate (Compound 11)

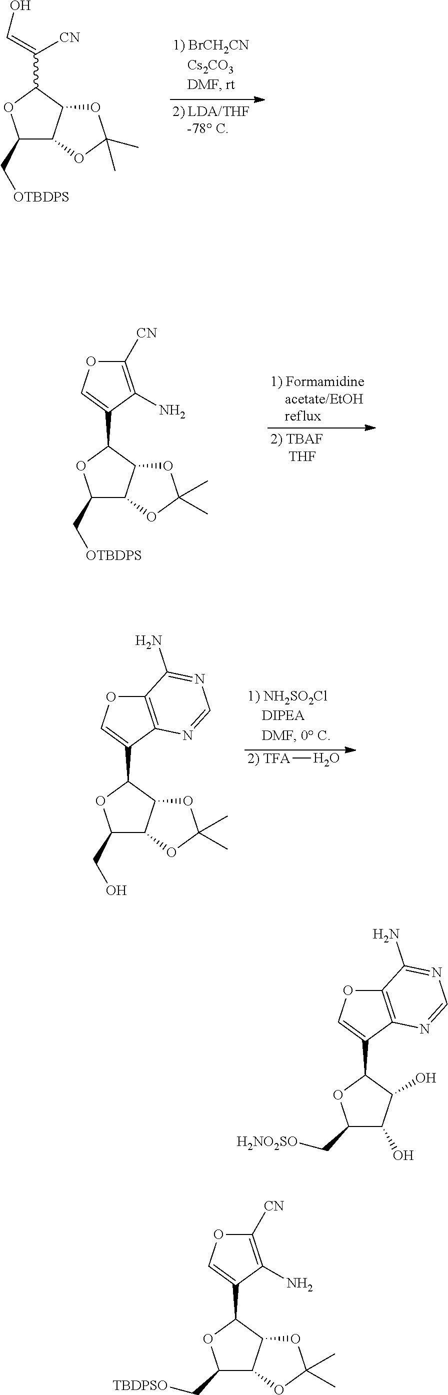

### 3-Amino-4-((3aS,4S,6R,6aR)-6-((tert-butyldiphenylsilyloxy)methyl)-2,2-dimethyltetrahydrofuro[3,4-d][1,3]dioxol-4-yl)furan-2-carbonitrile

2-((3aS,6R,6aR)-6-((tert-butyldiphenylsilyloxy)methyl)-2,2-dimethyltetrahydrofuro[3,4-d][1,3]dioxol-4-yl)-3-hydroxyacrylonitrile (500 mg, 1.04 mmol) is dissolved in anhydrous DMF (5 mL) along with bromoacetonitrile (149 mg, 1.25 mmol) and Cs₂CO₃ (407 mg, 1.25 mmol). The mixture is stirred at room temperature for 2 hours until TLC and LC-MS showed the reaction is complete. The mixture is diluted with EtOAc (80 mL), washed with water (30 mL×2) and brine (30 mL). The organic layer is separated, dried over Na₂SO₄ and filtered. After removal of the solvents, the residue is purified by column chromatography on ISCO system (eluted by 0-40% ethyl acetate in hexane) to give 2-((3aS,6R,6aR)-6-((tert-butyldiphenylsilyloxy)methyl)-2,2-dimethyltetrahydrofuro[3,4-d][1,3]dioxol-4-yl)-3-(cyanomethoxy)acrylonitrile (180 mg, 33%) as a mixture of isomers. LCMS (m/z) M+H=519.3; t_{R}=3.44, 3.59 min.

This product (170 mg, 0.33 mmol) and a magnetic stir bar are placed in a 25 mL round bottom flask. The flask is equipped with an adaptor and dried in vacuum for 2 hours. At −78° C., anhydrous THF (5 mL) is introduced with a syringe, and lithium diisopropylamide (0.33 mL, 2 M solution in THF, 0.66 mmol) is slowly added. After the mixture is stirred at −78° C. for another 30 minutes, the reaction is quenched by addition of de-ionized water (50 μL). Two isomers are found. The mixture is concentrated to dryness and the residue purified by silica gel column chromatography on ISCO system to give 3-Amino-4-((3aS,4S,6R,6aR)-6-((tert-butyldiphenylsilyloxy)methyl)-2,2-dimethyltetrahydrofuro[3,4-d][1,3]dioxol-4-yl)furan-2-carbonitrile (the less polar isomer, 34 mg, 20%). LCMS (m/z) M+H=519.3; t_{R}=3.74 min.

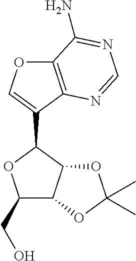

### ((3aR,4R,6S,6aS)-6-(4-aminofuro[3,2-d]pyrimidin-7-yl)-2,2-dimethyltetrahydrofuro[3,4-d][1,3]dioxol-4-yl)methanol

3-Amino-4-((3aS,4S,6R,6aR)-6-((tert-butyldiphenylsilyloxy)methyl)-2,2-dimethyltetrahydrofuro[3,4-d][1,3]dioxol-4-yl)furan-2-carbonitrile (15 mg, 0.029 mmol) and formamidine acetate (60 mg, 0.58 mmol) are dissolved in EtOH (2 mL). The solution is heated at reflux for 24 hrs. The reaction mixture is concentrated and the residue purified by preparative TLC to give 7-((3aS,4S,6R,6aR)-6-((tert-butyldiphenylsilyloxy)methyl)-2,2-dimethyltetrahydrofuro[3,4-d][1,3]dioxol-4-yl)furo[3,2-d]pyrimidin-4-amine (8.7 mg, 55%). LCMS (m/z) M+H=546.4; t_{R}=2.97 min.

This amino compound (7.0 mg, 0.013 mmol) in THF (0.5 mL) is treated with 1 M tetrabutylammonium fluoride solution in THF (0.5 mL) at room temperature until LCMS and TLC showed the reaction is complete. The mixture is concentrated and the residue purified by preparative TLC to give ((3aR,4R,6S,6aS)-6-(4-aminofuro[3,2-d]pyrimidin-7-yl)-2,2-dimethyltetrahydrofuro[3,4-d][1,3]dioxol-4-yl)methanol (3.0 mg, 76%). LCMS (m/z) M+H=308.2; t_{R}=0.24 min.

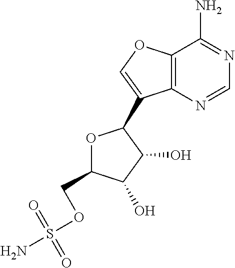

### ((2R,3S,4R,5S)-5-(4-aminofuro[3,2-d]pyrimidin-7-yl)-3,4-dihydroxytetrahydrofuran-2-yl)methyl sulfamate (Compound 11)

((3aR,4R,6S,6aS)-6-(4-aminofuro[3,2-d]pyrimidin-7-yl)-2,2-dimethyltetrahydrofuro[3,4-d][1,3]dioxol-4-yl)methanol (3.0 mg, 9.8 μmol) is dissolved in DMF (0.5 mL). To the solution is sequentially added N,N-diisopropylethylamine (0.1 mL) and 2 M aminosulfonyl chloride in acetonitrile (0.1 mL) at 0° C. then stirred for 1 hour. The mixture is concentrated to remove solvents, the residue is re-dissolved in EtOAc (4 mL) and washed with brine (2 mL). The organic layer is dried over Na₂SO₄, filtered and concentrated to afford a crude sulfamate product. LCMS (m/z) M+H=387.1; t_{R}=0.25 min.

The crude sulfamate from previous step is treated with a solution of 90% trifluoroacetic acid (0.45 mL trifluoroacetic acid and 0.05 mL water). The reaction mixture is stirred at room temperature for 30 min, concentrated, and the residue purified by preparative TLC to give the title compound (1.5 mg, 44% for 2 steps). LCMS (m/z) M+H=347.1; t_{R}=0.17 min.

### Example 18

### Preparation of ((2R,3S,4R,5S)-5-(4-((S)-2,3-dihydro-1H-inden-1-ylamino)furo[3,2-d]pyrimidin-7-yl)-3,4-dihydroxytetrahydrofuran-2-yl)methyl sulfamate (Compound 12)

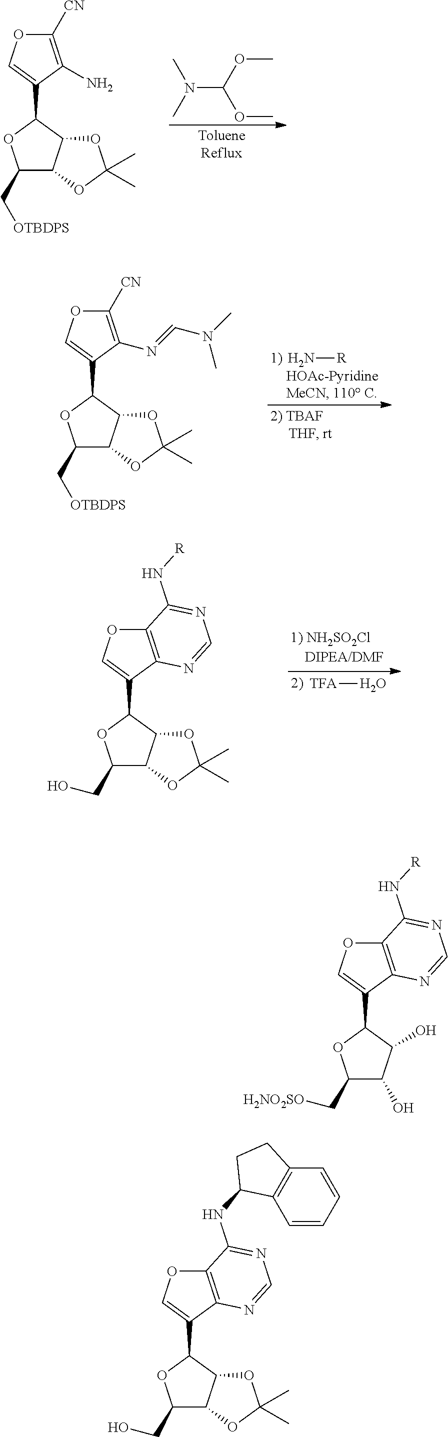

### ((3aR,4R,6S,6aS)-6-(4-((S)-2,3-dihydro-1H-inden-1-ylamino)furo[3,2-d]pyrimidin-7-yl)-2,2-dimethyltetrahydrofuro[3,4-d][1,3]dioxol-4-yl)methanol

3-Amino-4-((3aS,4S,6R,6aR)-6-((tert-butyldiphenylsilyloxy)methyl)-2,2-dimethyltetrahydrofuro[3,4-d][1,3]dioxol-4-yl)furan-2-carbonitrile (340 mg, 0.66 mmol) is dissolved in anhydrous toluene (10 mL). To this is added DMF dimethyl acetal (119 mg, 1.0 mmol). The mixture is then heated at 110° C. for 4 hours until TLC and LC-MS showed the reaction is complete. The mixture is concentrated and dried in vacuum to yield the crude (4-((3aS,4S,6R,6aR)-6-((tert-butyldiphenylsilyloxy)methyl)-2,2-dimethyltetrahydrofuro[3,4-d][1,3]dioxol-4-yl)-2-cyanofuran-3-yl)-N,N-dimethylformimidamide. LCMS (m/z) M+H=574.6; t_{R}=3.76 min.

In a microwave reaction vessel are placed the crude compound (376 mg, 0.66 mmol) from last step, a magnetic stir bar, and anhydrous acetonitrile (5 mL). The mixture is added (S)-1-aminoindane (439 mg, 3.3 mmol), pyridine (790 mg), HOAc (600 mg), and heated with microwave to 110° C. for 60 minutes. The reaction mixture is concentrated, diluted with EtOAc (40 mL), and washed with brine (15 mL×2). EtOAc layer is separated, dried, filtered and concentrated to dryness. The residue is purified by silica gel column chromatography to give 7-((3aS,4S,6R,6aR)-6-((tert-butyldiphenylsilyloxy)methyl)-2,2-dimethyltetrahydrofuro[3,4-d][1,3]dioxol-4-yl)-N—((S)-2,3-dihydro-1H-inden-1-yl)furo[3,2-d]pyrimidin-4-amine (249 mg, 57%). LCMS (m/z) M+H=662.6; t_{R}=3.81 min.

This compound (249 mg, 0.38 mmol) is dissolved in THF (1.0 mL). The mixture is added 1 M tetrabutylammonium fluoride solution in THF (0.5 mL), stirred at room temperature for 2 hours. The solvent is evaporated and the residue purified by preparative TLC to give ((3aR,4R,6S,6aS)-6-(4-((S)-2,3-dihydro-1H-inden-1-ylamino)furo[3,2-d]pyrimidin-7-yl)-2,2-dimethyl-tetrahydrofuro[3,4-d][1,3]dioxol-4-yl)methanol (121 mg, 75%). LCMS (m/z) M+H=424.5; t_{R}=1.80 min.

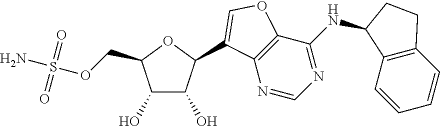

### ((2R,3S,4R,5S)-5-(4-((S)-2,3-dihydro-1H-inden-1-ylamino)furo[3,2-d]pyrimidin-7-yl)-3,4-dihydroxytetrahydrofuran-2-yl)methyl sulfamate (Compound 12)

((3aR,4R,6S,6aS)-6-(4-((S)-2,3-dihydro-1H-inden-1-ylamino)furo[3,2-d]pyrimidin-7-yl)-2,2-dimethyltetrahydrofuro[3,4-d][1,3]dioxol-4-yl)methanol (121 mg, 0.286 mmol), N,N-diisopropylethylamine (150 μL) are dissolved in anhydrous DMF (5 mL). The mixture is cooled to 0° C., slowly added a solution of 2 M NH₂SO₂Cl in acetonitrile (250 μL), stirred at 0° C. for 15 min, then graduated warmed to room temperature over 1 hr. The mixture is extracted by EtOAc—H₂O, the EtOAc layer is separated and concentrated to dryness to afford a crude sulfamate product. LCMS (m/z) M+H=503.4; t_{R}=1.92 min.

The crude product from previous step is dissolved in a 7:3 TFA-H₂O solution (5 mL). The mixture is stirred at room temperature for 2 hours, concentrated, dried in vacuum, and the residue purified by preparative TLC to give the final title compound (54 mg, 41% for 2 steps). LCMS (m/z) M+H=463.4; t_{R}=2.07 min; ¹H NMR (DMSO-d₆, 500 MHz) δ 8.38 (s, 1H), 8.29 (broad d, J=5.7 Hz, 1H), 8.17 (s, 1H), 7.57 (s, 2H), 7.28 (d, J=7.4 Hz, 1H), 7.25-7.18 (m, 2H), 7.14 (d, J=7.4 Hz, 1H), 5.88 (q, J=7.9 Hz, 1H), 5.33 (d, J=5.2 Hz, 1H), 5.16 (d, J=5.9 Hz, 1H), 4.92 (d, J=4.3 Hz, 1H), 4.34 (q, J=4.8 Hz, 1H), 4.28 (dd, J=10.7, 3.3 Hz, 1H), 4.16-4.08 (m, 3H), 4.05-4.01 (m, 1H), 3.05-2.98 (m, 1H), 2.90-2.82 (m, 1H), 2.54-2.46 (m, 1H), 2.11-2.02 (m, 1H).

### Example 19

### Preparation of ((2R,3S,4R,5S)-5-(4-aminopyrazolo[1,5-a][1,3,5]triazin-8-yl)-3,4-dihydroxytetrahydrofuran-2-yl)methyl sulfamate (Compound 13)

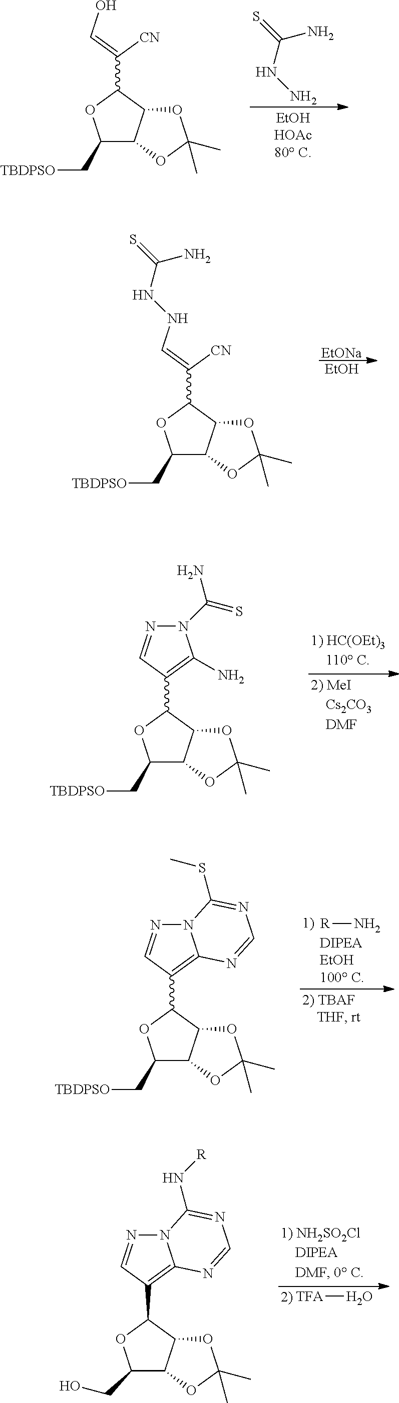

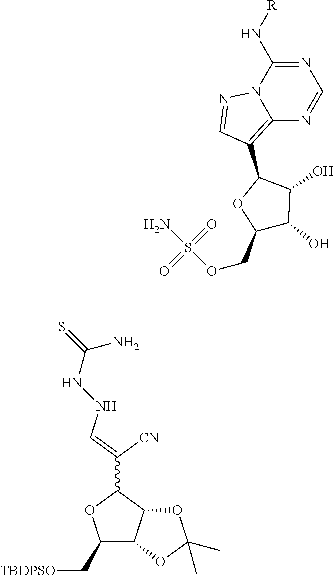

### 2-(2-((3aS,6R,6aR)-6-((tert-butyldiphenylsilyloxy)methyl)-2,2-dimethyltetrahydrofuro[3,4-d][1,3]dioxol-4-yl)-2-cyanovinyl)hydrazinecarbothioamide

A mixture of 2-((3aS,6R,6aR)-6-((tert-butyldiphenylsilyloxy)methyl)-2,2-dimethyl-tetrahydrofuro[3,4-d][1,3]dioxol-4-yl)-3-hydroxyacrylonitrile (480 mg, 1.00 mmol), thiosemicarbazide (138 mg, 1.50 mmol), AcOH (0.175 mL), and water (1 mL) in EtOH (3 mL) is heated at 80° C. for 2 hrs. The solvents are removed by rotary evaporation. The residue is purified by silica gel column chromatography to give 2-(2-((3aS,6R,6aR)-6-((tert-butyldiphenylsilyloxy)methyl)-2,2-dimethyltetrahydrofuro[3,4-d][1,3]dioxol-4-yl)-2-cyanovinyl)hydrazinecarbothioamide (337 mg, 61%). LCMS (m/z) M+H=553.5; t_{R}=3.39 min.

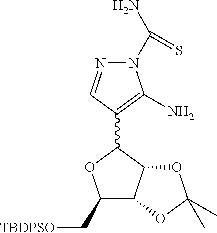

### 5-Amino-4-((3aS,6R,6aR)-6-((tert-butyldiphenylsilyloxy)methyl)-2,2-dimethyltetrahydrofuro[3,4-d][1,3]dioxol-4-yl)-1H-pyrazole-1-carbothioamide

2-(2-((3aS,6R,6aR)-6-((tert-butyldiphenylsilyloxy)methyl)-2,2-dimethyl-tetrahydrofuro[3,4-d][1,3]dioxol-4-yl)-2-cyanovinyl)hydrazinecarbothioamide (330 mg, 0.60 mmol) is dissolved in EtOH (2.5 mL). To this is added dropwise a solution of EtONa in EtOH (0.60 mL, 1 M). The resulting mixture is stirred at room temperature for 30 min, acidified by HOAc (120 mg), the solvents are evaporated under vacuum, and the residue purified by silica gel column chromatography to give 5-Amino-4-((3aS,6R,6aR)-6-((tert-butyldiphenylsilyloxy)methyl)-2,2-dimethyltetrahydrofuro[3,4-d][1,3]dioxol-4-yl)-1H-pyrazole-1-carbothioamide as a white solid (mixture of 2 isomers, 264 mg, 80%). LCMS (m/z) M+H=553.5; t_{R}=3.59, 3.61 min.

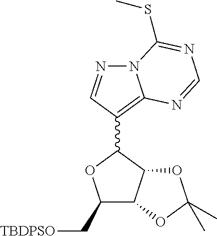

### 8-((3aS,6R,6aR)-6-((tert-butyldiphenylsilyloxy)methyl)-2,2-dimethyltetrahydrofuro[3,4-d][1,3]dioxol-4-yl)-4-(methylthio)pyrazolo[1,5-a][1,3,5]triazine

A mixture of 5-Amino-4-((3aS,6R,6aR)-6-((tert-butyldiphenylsilyloxy)methyl)-2,2-dimethyltetrahydrofuro[3,4-d][1,3]dioxol-4-yl)-1H-pyrazole-1-carbothioamide (210 mg, 0.38 mmol) and HC(OEt)₃ (2 mL) is heated at 110° C. for 2 hrs. After cooling to room temperature, the mixture is concentrated under high vacuum, and the residue purified by silica gel column chromatography to give 8-((3aS,4S,6R,6aR)-6-((tert-butyldiphenylsilyloxy)methyl)-2,2-dimethyltetrahydrofuro[3,4-d][1,3]dioxol-4-yl)pyrazolo[1,5-a][1,3,5]triazine-4(3H)-thione (containing two isomers, 96 mg, 45%). LCMS (m/z) M+H=563.5; t_{R}=3.65 min.

A mixture of this product (80 mg, 0.14 mmol), MeI (99 mg, 0.70 mmol) and Cs₂CO₃ (46 mg, 0.14 mmol) in anhydrous DMF (2 mL) is stirred at room temperature for 2 hrs. The reaction mixture is diluted with EtOAc (50 mL) and washed with water (25 mL×2) and brine (25 mL). The organic layer is separated, dried over Na₂SO₄, filtered, concentrated, and the residue purified by silica gel column chromatography to give 8-((3aS,6R,6aR)-6-((tert-butyldiphenylsilyloxy)methyl)-2,2-dimethyltetrahydrofuro[3,4-d][1,3]dioxol-4-yl)-4-(methylthio)pyrazolo[1,5-a][1,3,5]triazine (two isomers, 70 mg, 85%). LCMS (m/z) M+H=577.5; t_{R}=4.24 min.

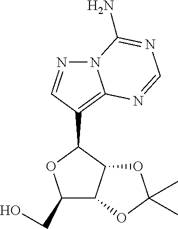

### ((3aR,4R,6S,6aS)-6-(4-aminopyrazolo[1,5-a][1,3,5]triazin-8-yl)-2,2-dimethyltetrahydrofuro[3,4-d][1,3]dioxol-4-yl)methanol

In a 10 mL microwave reaction vessel are placed 8-((3aS,6R,6aR)-6-((tert-butyldiphenylsilyloxy)methyl)-2,2-dimethyltetrahydrofuro[3,4-d][1,3]dioxol-4-yl)-4-(methylthio)pyrazolo[1,5-a][1,3,5]triazine (35 mg, 0.061 mmol), ammonium solution in MeOH (1.5 mL, 7 M), and a magnetic stir bar. The mixture is heated at 100° C. under microwave for 4 hours. The solvents are evaporated and the residue purified by preparative TLC to give the amino product 8-((3aS,6R,6aR)-6-((tert-butyldiphenylsilyloxy)methyl)-2,2-dimethyl-tetrahydrofuro[3,4-d][1,3]dioxol-4-yl)pyrazolo[1,5-a][1,3,5]triazin-4-amine (11 mg, 33%). LCMS (m/z) M+H=546.4; t_{R}=3.34 min.

A mixture of this amino product (9.0 mg, 0.016 mmol) in THF (0.5 mL) is treated with tetrabutylammonium fluoride solution in THF (0.5 mL, 1 M) at room temperature. After LCMS and TLC showed the reaction is complete, the mixture is concentrated, and the residue purified by preparative TLC to give ((3aR,4R,6S,6aS)-6-(4-aminopyrazolo[1,5-a][1,3,5]triazin-8-yl)-2,2-dimethyltetrahydrofuro[3,4-d][1,3]dioxol-4-yl)methanol (3.3 mg, 65%). LCMS (m/z) M+H=308.2; t_{R}=1.10 min.

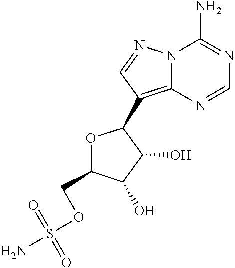

### ((2R,3S,4R,5S)-5-(4-aminopyrazolo[1,5-a][1,3,5]triazin-8-yl)-3,4-dihydroxytetrahydrofuran-2-yl)methyl sulfamate (Compound 13)

((3aR,4R,6S,6aS)-6-(4-aminopyrazolo[1,5-a][1,3,5]triazin-8-yl)-2,2-dimethyltetrahydrofuro[3,4-d][1,3]dioxol-4-yl)methanol (3.3 mg, 10.7 μmol) is dissolved in DMF (0.5 mL). The mixture is sequentially added N,N-diisopropylethylamine (0.1 mL), aminosulfonyl chloride (0.1 mL, 2 M solution in acetonitrile) at 0° C., then stirred at 0° C. for 1 hour until LCMS showed the reaction is complete. The mixture is concentrated in vacuum, re-dissolved in EtOAc (4 mL) and washed with brine (2 mL). The organic layer is dried over Na₂SO₄, filtered, concentrated to give the crude sulfamate LCMS (m/z) M+H=387.1; t_{R}=1.44 min.

The crude sulfamate product from previous step is added a 90% trifluoroacetic acid solution (prepared from 0.45 mL trifluoroacetic acid and 0.05 mL water), the resulting mixture is stirred at room temperature for 30 min until LCMS showed the reaction is complete. The mixture is concentrated, and the residue purified by preparative TLC to give the title compound (1.0 mg, 27% for 2 steps). LCMS (m/z) M+H=347.1; t_{R}=0.35 min.

### Example 20

### Preparation of ((2R,3S,4R,5S)-5-(4-((S)-2,3-dihydro-1H-inden-1-ylamino)pyrazolo[1,5-a][1,3,5]triazin-8-yl)-3,4-dihydroxytetrahydrofuran-2-yl)methyl sulfamate (Compound 14)

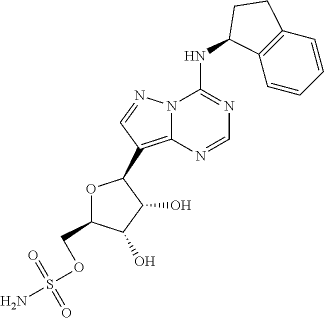

### ((2R,3S,4R,5S)-5-(4-((S)-2,3-dihydro-1H-inden-1-ylamino)pyrazolo[1,5-a][1,3,5]triazin-8-yl)-3,4-dihydroxytetrahydrofuran-2-yl)methyl sulfamate (Compound 14)

The title compound is prepared similarly from 8-((3aS,6R,6aR)-6-((tert-butyldiphenylsilyloxy)methyl)-2,2-dimethyltetrahydrofuro[3,4-d][1,3]dioxol-4-yl)-4-(methylthio)pyrazolo[1,5-a][1,3,5]triazine (35 mg, 0.061 mmol) and (S)-1-aminoindane (16 mg, 0.122 mmol) in 4 steps and a 13% overall yield, affording 2.6 mg of the product. LCMS (m/z) M+H=463.4; t_{R}=2.56 min.

### Example 21

### Preparation of (1R,4S)-2-(benzyloxymethyl)-4-(4-((S)-2,3-dihydro-1H-inden-1-ylamino)pyrazolo[1,5-a][1,3,5]triazin-8-yl)cyclopent-2-enol

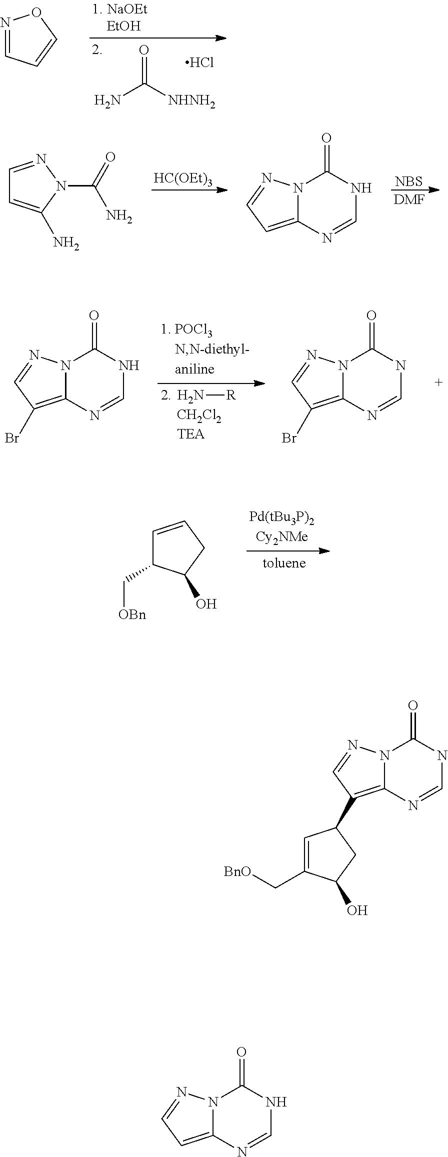

### Pyrazolo[1,5-a][1,3,5]triazin-4(3H)-one

To a stirring solution of isoxazole (35.0 g, 506.0 mmol) in ethanol (200 mL) cooled at 0° C. is slowly added sodium ethoxide (197 mL, 21% in ethanol, 532 mmol). The reaction mixture is stirred for an additional 30 minutes after the addition of sodium ethoxide. The solvent is removed in vacuo followed by the addition of water (200 mL) and neutralized with acetic acid to pH˜7. The mixture is added semicarbazide hydrochloride (56.43 g, 506 mmol), stirred at 25° C. for 16 hours, neutralized with NaOH (1 N) to pH˜7, and then extracted with chloroform (5×). The organic layers are dried over Na₂SO₄, filtered, concentrated, and purified via chromatography eluted by a gradient of hexanes-EtOAc to give 5-amino-1H-pyrazole-1-carboxamide as a white solid (13.8 g, 21.6%). TLC R_{f}=0.51 (1:1 EtOAc:hexanes).

A solution of 5-amino-1H-pyrazole-1-carboxamide and ethyl orthoformate is heated at 110° C. for 15 hours. The reaction mixture is concentrated to dryness and triturated with hexanes. The solid is then dried under vacuum to afford pyrazolo[1,5-a][1,3,5]triazin-4(3H)-one (8.23 g, 76.6%) as a tan solid. LCMS (m/z): M+H=137.1; t_{R}=0.22 min; TLC R_{f}=0.44 (9:1 DCM:MeOH).

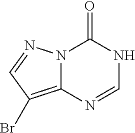

### 8-Bromopyrazolo[1,5-a][1,3,5]triazin-4(3H)-one

To a solution of pyrazolo[1,5-a][1,3,5]triazin-4(3H)-one (8.00 g, 58.7 mmol) in DMF (80 mL) is added N-bromosuccinimide (10.46 g, 58.7 mmol) and the mixture stirred at 40° C. for 5 hours. The reaction mixture is concentrated to dryness and purified via silica chromatography using a gradient of MeOH (up to 5%) in dichloromethane to give 8-bromopyrazolo[1,5-a][1,3,5]triazin-4(3H)-one (11.2 g, 88.7% yield) as a tan solid. LCMS (m/z): M+H=215.1, 217.1; t_{R}=2.36 min; TLC R_{f}=0.18 (19:1 DCM:MeOH).

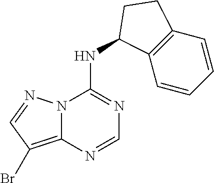

### (S)-8-Bromo-N-(2,3-dihydro-1H-inden-1-yl)pyrazolo[1,5-a][1,3,5]triazin-4-amine

A solution of 8-bromopyrazolo[1,5-a][1,3,5]triazin-4(3H)-one (5.00 g, 23.2 mmol) in phosphorous oxychloride (40 mL) and diethylaniline (15 mL) is heated at reflux for 2 hours. The solvent is removed under reduced pressure, and the residue is taken up in dichloromethane (50 mL) and slowly added to a solution of (S)-1-aminoindane (4.46 mL, 34.8 mmol) and triethylamine (16.1 mL, 116 mmol) in dichloromethane (100 mL). The reaction mixture is stirred at 25° C. for 16 hours. The reaction mixture is concentrated then purified via chromatography using a gradient of hexanes to EtOAc to afford (S)-8-Bromo-N-(2,3-dihydro-1H-inden-1-yl)pyrazolo[1,5-a][1,3,5]triazin-4-amine (2.70 g, 35%) as a yellow solid. LCMS (m/z): M+H=330.3, 332.3, t_{R}=2.36 min; TLC R_{f}=0.42 (1:4 EtOAc:hexanes); ¹H NMR (DMSO-d₆, 500 MHz) δ 9.34 (d, J=2.5 Hz, 1H), 8.26 (s, 1H), 8.19 (s, 1H), 7.18 (m, 3H), 7.08 (m, 1H), 5.80 (m, 1H), 2.98 (m, 1H), 2.80 (m, 1H), 2.41 (m, 1H), 2.21 (m, 1H).

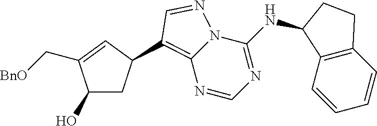

### (1R,4S)-2-(benzyloxymethyl)-4-(4-((S)-2,3-dihydro-1H-inden-1-ylamino)pyrazolo[1,5-a][1,3,5]triazin-8-yl)cyclopent-2-enol

To a solution of (S)-8-Bromo-N-(2,3-dihydro-1H-inden-1-yl)pyrazolo[1,5-a][1,3,5]triazin-4-amine (1.00 g, 3.02 mmol), (1R,2S)-2-(benzyloxymethyl)cyclopent-3-enol (0.925 g, 4.53 mmol), and (1.29 mL, 6.04 mmol) N,N-dicyclohexylmethylamine in toluene (50 mL) is added Pd(tBu₃P)₂ (0.385 g, 0.755 mmol) and the mixture stirred at 65° C. for 48 hours. The reaction mixture is concentrated to dryness and purified via chromatography using a gradient of hexanes to EtOAc to afford the title compound (0.350 g, 25.5%) as a yellow solid. LCMS (m/z): M+H=454.5; t_{R}=8.10 min with the long method; TLC R_{f}=0.30 (1:1 EtOAc:hexanes).

### Example 22

### Preparation of ((1R,2S,4R)-4-(4-((S)-2,3-dihydro-1H-inden-1-ylamino)pyrazolo[1,5-a][1,3,5]triazin-8-yl)-2-hydroxycyclopentyl)methyl sulfamate (Compound 15)

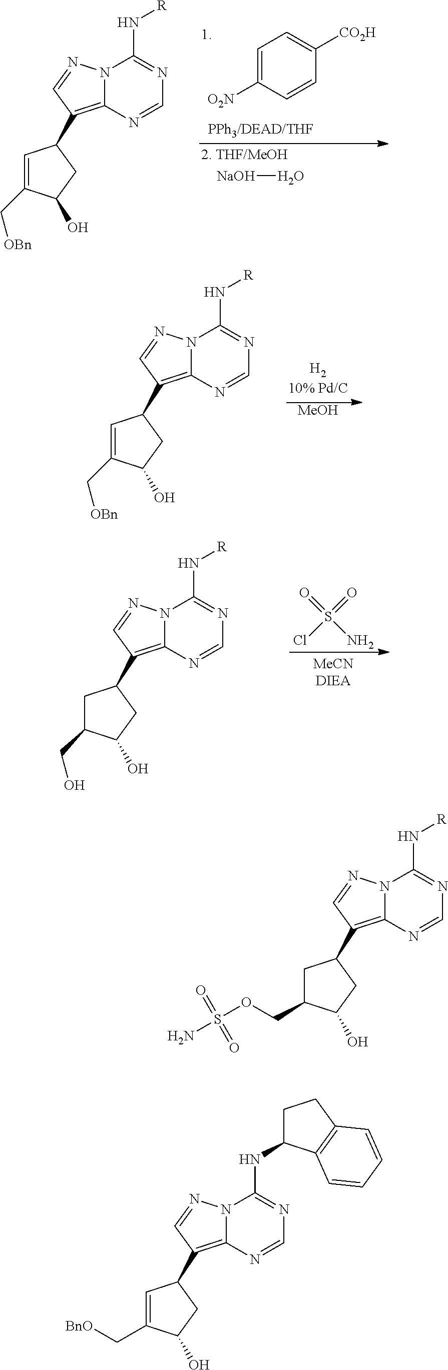

### (1S,4S)-2-(benzyloxymethyl)-4-(4-((S)-2,3-dihydro-1H-inden-1-ylamino)pyrazolo[1,5-a][1,3,5]triazin-8-yl)cyclopent-2-enol

To a solution of (1R,4S)-2-(benzyloxymethyl)-4-(4-((S)-2,3-dihydro-1H-inden-1-ylamino)pyrazolo[1,5-a][1,3,5]triazin-8-yl)cyclopent-2-enol (0.300 g, 0.661 mmol), 4-nitrobenzoic acid (0.132 g, 0.793 mmol), and triphenyphosphine (0.207 g, 0.793 mmol) in THF (5 mL) is added diethyl azodicarboxylate (0.361 mL, 0.793 mmol) and the reaction mixture stirred at 25° C. for 2 hours. The mixture is concentrated to dryness and the intermediate purified via chromatography using a gradient of hexanes to EtOAc. The solid is taken up in THF/MeOH/H₂O (8:3:3) and sodium hydroxide (0.052 g, 1.32 mmol) is added to the solution. The mixture is stirred at 25° C. for 2 hours, concentrated, and purified via chromatography using a gradient of hexanes to EtOAc to afford (1S,4S)-2-(benzyloxymethyl)-4-(4-((S)-2,3-dihydro-1H-inden-1-ylamino)pyrazolo[1,5-a][1,3,5]triazin-8-yl)cyclopent-2-enol (0.148 g, 49.3%) as a yellow solid. LCMS (m/z): M+H=454.5; t_{R}=7.69 min with the long method; TLC R_{f}=0.23 (EtOAc).

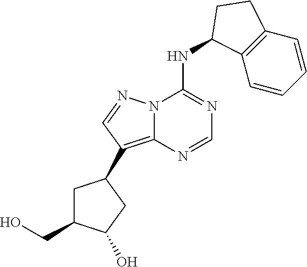

### (1S,2R,4R)-4-(4-((S)-2,3-dihydro-1H-inden-1-ylamino)pyrazolo[1,5-a][1,3,5]triazin-8-yl)-2-(hydroxymethyl)cyclopentanol

A solution of (1S,4S)-2-(benzyloxymethyl)-4-(4-((S)-2,3-dihydro-1H-inden-1-ylamino)pyrazolo[1,5-a][1,3,5]triazin-8-yl)cyclopent-2-enol (0.148 g, 0.326 mmol) in methanol (25 mL) containing 10% Pd on carbon (0.200 g) is hydrogenated at 60 psi in a PARR apparatus for 48 hours. The reaction mixture is filtered through celite, concentrated to dryness and purified via chromatography using a gradient of 0-10% MeOH in dichloromethane to afford (0.030 g, 25.2%) of (1S,2R,4R)-4-(4-((S)-2,3-dihydro-1H-inden-1-ylamino)pyrazolo[1,5-a][1,3,5]triazin-8-yl)-2-(hydroxymethyl)cyclopentanol as a white solid. LCMS (m/z): M+H=366.4; t_{R}=6.12 min with the long method; TLC R_{f}=0.20 (19:1 DCM:MeOH); ¹H NMR (DMSO-d₆, 500 MHz) δ 9.05 (d, J=8.6 Hz, 1H), 8.13 (s, 1H), 8.06 (s, 1H), 7.27 (m, 1H), 7.22 (m, 2H), 7.14 (m, 1H), 5.85 (m, 1H), 4.53 (m, 2H), 4.00 (m, 1H), 3.46 (m, 2H), 3.35 (m, 1H), 3.04 (ddd, J=3.0, 8.9, 15.5 Hz, 1H), 2.86 (m, 1H), 2.47 (m, 1H), 2.27 (m, 1H), 2.18 (m, 1H) 1.98 (m, 1H), 1.89 (m, 2H), 1.40 (m, 1H); ¹³C NMR (DMSO-d₆, 125 MHz) δ 152.1, 149.1, 145.2, 143.4, 143.1, 143.0, 127.6, 126.3, 124.6, 123.8, 113.2, 73.3, 63.5, 55.2, 51.1, 42.2, 35.7, 31.5, 31.3, 29.8.

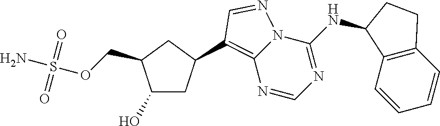

### ((1R,2S,4R)-4-(4-((S)-2,3-dihydro-1H-inden-1-ylamino)pyrazolo[1,5-a][1,3,5]triazin-8-yl)-2-hydroxycyclopentyl)methyl sulfamate (Compound 15)

To a solution of (1S,2R,4R)-4-(4-((S)-2,3-dihydro-1H-inden-1-ylamino)pyrazolo[1,5-a][1,3,5]triazin-8-yl)-2-(hydroxymethyl)cyclopentanol (0.030 g, 0.082 mmol) and N,N-diisopropylethylamine (0.042 mL, 0.246 mmol) in acetonitrile (3.0 mL) cooled at 0° C. is added sulfamoyl chloride (0.010 g, 0.0902 mmol). The reaction mixture is allowed to warm to 25° C. and stirred for 4 hours. The mixture is concentrated, and purified via silica chromatography using dichloromethane/EtOAc/MeOH (1:1:0.1) to afford the title compound (0.005 g, 20.3%) as a white solid. LCMS (m/z): M+H=445.4; t_{R}=7.06 min with the long method; TLC R_{f}=0.30 (1:1:0.1 EtOAc:DCM:MeOH); ¹H NMR (DMSO-d₆, 500 MHz) δ 9.08 (d, J=8.6 Hz, 1H), 8.14 (s, 1H), 8.08 (s, 1H), 7.46 (broad, 2H), 7.28 (m, 1H), 7.23 (m, 2H), 7.14 (m, 1H), 5.85 (m, 1H), 4.83 (d, J=4.3 Hz, 1H), 4.12 (dd, J=6.2, 9.5 Hz, 1H), 4.01 (m, 2H), 3.50 (m, 1H), 3.05 (ddd, J=3.1, 8.9, 15.8 Hz, 1H), 2.87 (m, 1H), 2.47 (m, 1H), 2.27 (m, 2H), 2.21 (m, 1H) 1.96 (m, 2H), 1.48 (m, 1H); ¹³C NMR (DMSO-d₆, 125 MHz) δ 152.2, 149.1, 145.2, 143.4, 143.1, 143.0, 127.6, 126.3, 124.6, 123.8, 112.6, 72.8, 70.8, 55.2, 47.6, 42.0, 35.4, 31.5, 31.2, 29.8.

### Example 23

### Preparation of 7-((3aS,6R,6aR)-6-((tert-butyldiphenylsilyloxy)methyl)-2,2-dimethyltetrahydrofuro[3,4-d][1,3]dioxol-4-yl)-N—((S)-2,3-dihydro-1H-inden-1-yl)-5H-pyrrolo[3,2-d]pyrimidin-4-amine

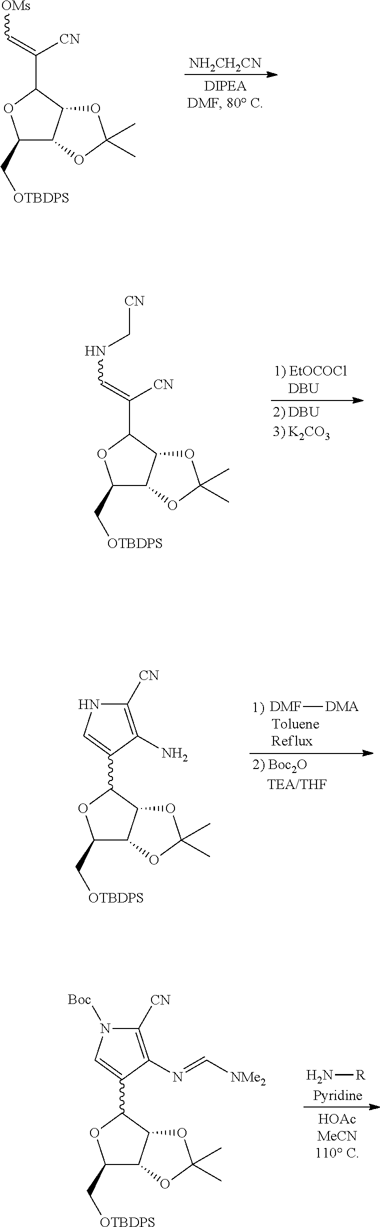

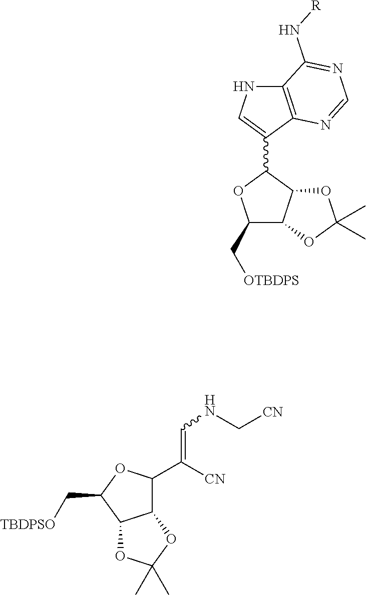

### 2-((3aS,6R,6aR)-6-((tert-butyldiphenylsilyloxy)methyl)-2,2-dimethyltetrahydrofuro[3,4-d][1,3]dioxol-4-yl)-3-(cyanomethylamino)acrylonitrile

2-((3aS,6R,6aR)-6-((tert-butyldiphenylsilyloxy)methyl)-2,2-dimethyltetrahydrofuro[3,4-d][1,3]dioxol-4-yl)-2-cyanovinyl methanesulfonate (1.50 g, 2.69 mmol) is dissolved in anhydrous DMF (20 mL). To the solution is added 2-aminoacetonitrile (0.753 g, 13.45 mmol). The mixture is heated at 80° C. under N₂ for 4 hours, cooled, diluted with EtOAc (50 mL), and washed with water (25 mL×2) and brine (25 mL). The organic layer is separated, dried over Na₂SO₄ and concentrated to dryness. The residue is purified by silica gel column chromatography to give 2-((3aS,6R,6aR)-6-((tert-butyldiphenylsilyloxy)methyl)-2,2-dimethyltetrahydrofuro[3,4-d][1,3]dioxol-4-yl)-3-(cyanomethylamino)acrylonitrile (919 mg, 66%). LCMS (m/z) M+H=518.3; t_{R}=3.42, 3.56 min.

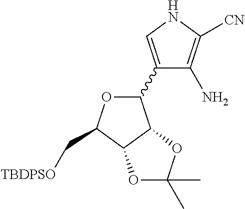

### 3-Amino-4-(3aS,6R,6aR)-6-((tert-butyldiphenylsilyloxy)methyl)-2,2-dimethyltetrahydrofuro[3,4-d][1,3]dioxol-4-yl)-1H-pyrrole-2-carbonitrile

To a solution of 2-((3aS,6R,6aR)-6-((tert-butyldiphenylsilyloxy)methyl)-2,2-dimethyltetrahydrofuro[3,4-d][1,3]dioxol-4-yl)-3-(cyanomethylamino)acrylonitrile (900 mg, 1.74 mmol) in dichloromethane (15 mL) is added ethyl chloroformate (208 mg, 1.92 mmol) and 1,8-diazabicyclo[5.4.0]undec-7-ene (DBU) (292 mg, 1.92 mmol). The mixture is stirred at room temperature for 1 hour. Then more DBU (292 mg, 1.92 mmol) is added, and the mixture stirred for another 2 hours. The mixture is washed with water (15 mL) and dried over Na₂SO₄. After removal of the solvents, the residue is dissolved in EtOH (5 mL). To the solution is added K₂CO₃ (483 mg, 3.5 mmol), and the resulting mixture is stirred at room temperature for 2 hours until LCMS showed the reaction is complete. The mixture is filtered, concentrated, and taken up into a mixture of EtOAc (20 mL) and H₂O (10 mL). The EtOAc layer is separated, dried, and concentrated. The residue is purified by silica gel column chromatography to give 3-amino-4-((3aS,6R,6aR)-6-((tert-butyldiphenylsilyloxy)methyl)-2,2-dimethyltetrahydrofuro[3,4-d][1,3]dioxol-4-yl)-1H-pyrrole-2-carbonitrile (432 mg, 48%). LCMS (m/z) M+H=518.3; t_{R}=3.49, 3.59 min

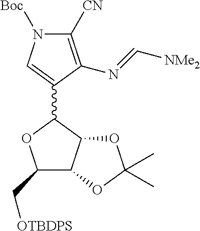

### tert-butyl 4-((3aS,6R,6aR)-6-((tert-butyldiphenylsilyloxy)methyl)-2,2-dimethyltetrahydrofuro[3,4-d][1,3]dioxol-4-yl)-2-cyano-3-((E)-(dimethylamino)methyleneamino)-1H-pyrrole-1-carboxylate

3-amino-4-((3aS,6R,6aR)-6-((tert-butyldiphenylsilyloxy)methyl)-2,2-dimethyltetrahydrofuro[3,4-d][1,3]dioxol-4-yl)-1H-pyrrole-2-carbonitrile (216 mg, 0.417 mmol) is dissolved in anhydrous toluene (6 mL). To the solution is added DMF dimethyl acetal (75 mg, 0.63 mmol). The mixture is heated at 110° C. for 4 hours, concentrated, dried in vacuum to give crude (E)-N′-(4-((3aS,6R,6aR)-6-((tert-butyldiphenylsilyloxy)methyl)-2,2-dimethyltetrahydrofuro[3,4-d][1,3]dioxol-4-yl)-2-cyano-1H-pyrrol-3-yl)-N,N-dimethylformimidamide. LCMS (m/z) M+H=573.5; t_{R}=3.02 min.

To a solution of the crude product from last step in dichloromethane (3 mL) are added Et₃N (64 mg, 0.63 mmol), 4-dimethylaminopyridine (10 mg, 0.08 mmol), and BOC anhydride (109 mg, 0.50 mmol). The mixture is stirred at room temperature overnight. The solvents are evaporated and the residue is purified by column chromatography to give tert-butyl 4-((3aS,6R,6aR)-6-((tert-butyldiphenylsilyloxy)methyl)-2,2-dimethyltetrahydrofuro[3,4-d][1,3]dioxol-4-yl)-2-cyano-3-((E)-(dimethylamino)methyleneamino)-1H-pyrrole-1-carboxylate as a white solid (259 mg, 92%). LCMS (m/z) M+H=673.6; t_{R}=3.79, 3.89 min.

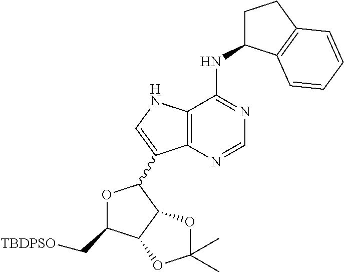

### 7-((3aS,6R,6aR)-6-((tert-butyldiphenylsilyloxy)methyl)-2,2-dimethyltetrahydrofuro[3,4-d][1,3]dioxol-4-yl)-N—((S)-2,3-dihydro-1H-inden-1-yl)-5H-pyrrolo[3,2-d]pyrimidin-4-amine

In a 10 mL microwave reaction vessel are placed tert-butyl 4-((3aS,6R,6aR)-6-((tert-butyldiphenylsilyloxy)methyl)-2,2-dimethyltetrahydrofuro[3,4-d][1,3]dioxol-4-yl)-2-cyano-3-((E)-(dimethylamino)methyleneamino)-1H-pyrrole-1-carboxylate (100 mg, 0.149 mmol), a magnetic stir bar, and anhydrous acetonitrile (1.5 mL). To the solution are added (S)-1-aminoindane (99 mg, 0.75 mmol), pyridine (237 mg) and acetic acid (180 mg). The resulting mixture is heated with microwave to 110° C. for 60 minutes. The reaction mixture is concentrated, re-dissolved in EtOAc (10 mL), and washed with brine (5 mL×2). The organic layer is separated, dried, and concentrated to give crude title compound and is used as such in the next step. LCMS (m/z) M+H=661.5; t_{R}=2.77 min.

### Example 24

### Preparation of ((2R,3S,4R,5S)-5-(4-((S)-2,3-dihydro-1H-inden-1-ylamino)-5H-pyrrolo[3,2-d]pyrimidin-7-yl)-3,4-dihydroxytetrahydrofuran-2-yl)methyl sulfamate (Compound 16)

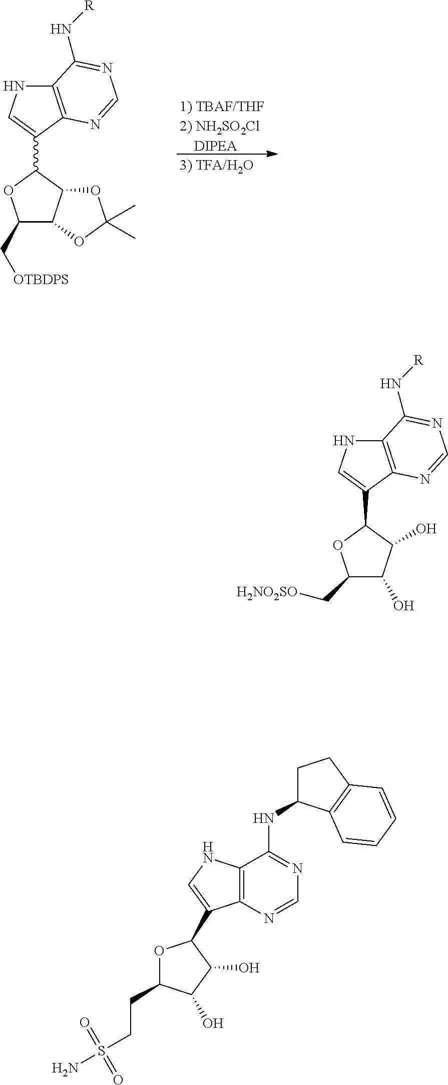

### ((2R,3S,4R,5S)-5-(4-((S)-2,3-dihydro-1H-inden-1-ylamino)-5H-pyrrolo[3,2-d]pyrimidin-7-yl)-3,4-dihydroxytetrahydrofuran-2-yl)methyl sulfamate (Compound 16)

Crude 7-((3aS,6R,6aR)-6-((tert-butyldiphenylsilyloxy)methyl)-2,2-dimethyl-tetrahydrofuro[3,4-d][1,3]dioxol-4-yl)-N—((S)-2,3-dihydro-1H-inden-1-yl)-5H-pyrrolo[3,2-d]pyrimidin-4-amine is dissolved in THF (1 mL). To this is added tetrabutylammonium fluoride (0.5 mL, 1 M solution in THF). The mixture is stirred at room temperature for 2 hours until LC-MS showed the reaction is complete. The solvent is evaporated and the residue is purified by preparative TLC to give ((3aR,4R,6S,6aS)-6-(4-((S)-2,3-dihydro-1H-inden-1-ylamino)-5H-pyrrolo[3,2-d]pyrimidin-7-yl)-2,2-dimethyltetrahydrofuro[3,4-d][1,3]dioxol-4-yl)methanol (14 mg, 22% for 2 steps). LCMS (m/z) M+H=423.4; t_{R}=1.75 min.

The product from last step (14 mg, 0.033 mmol), N,N-diisopropylethylamine (100 μL) are dissolved in anhydrous DMF (1 mL). The mixture is cooled to 0° C., and a solution of 2 M sulfamoyl chloride in acetonitrile (150 μL) is slowly added. The mixture is stirred at 0° C. for 15 min and then graduated warmed to room temperature over 1 hr. The mixture is extracted with EtOAc—H₂O, the EtOAc layer is separated, and concentrated to give crude ((3aR,4R,6S,6aS)-6-(4-((S)-2,3-dihydro-1H-inden-1-ylamino)-5H-pyrrolo[3,2-d]pyrimidin-7-yl)-2,2-dimethyltetrahydrofuro[3,4-d][1,3]dioxol-4-yl)methyl sulfamate. LCMS (m/z) M+H=502.3; t_{R}=1.84 min.

The crude sulfamate is dissolved in a 7:3 TFA-H₂O solution (1.5 mL), and the mixture is stirred at room temperature for 2 hours. The mixture is concentrated and purified by preparative TLC to give the title compound (6 mg, 39% for 2 steps). LCMS (m/z) M+H=462.3; t_{R}=1.52 min.

### Example 25

### Preparation of 2-((3S,4R)-3-(benzyloxymethyl)-4-(tert-butyldiphenylsilyloxy)-cyclopentyl)-3-hydroxyacrylonitrile

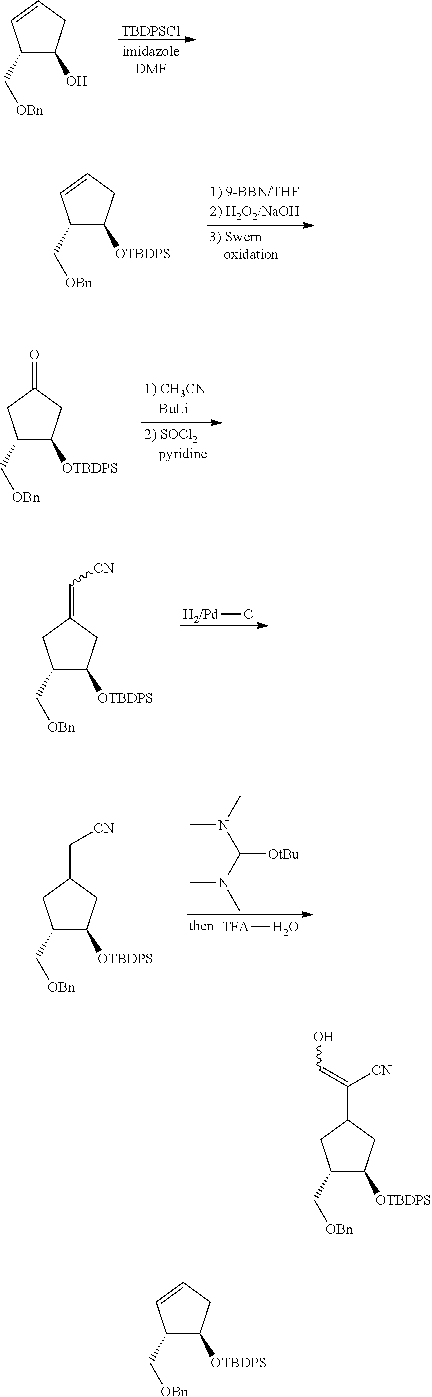

### ((1R,2S)-2-(benzyloxymethyl)cyclopent-3-enyloxy)(tert-butyl)diphenylsilane

A stirred mixture of (1R,2S)-2-(benzyloxymethyl)cyclopent-3-en-1-ol (4 g) and imidazole (2 g) in DMF (20 mL) in an ice-water bath is added TBDPSCl (5.6 mL) dropwise. The resulting mixture is then warmed to rt and stirred for 3 hr until TLC shows most of the starting material disappears. Ethyl acetate (80 mL) and water (50 mL) are added into the mixture, the organic layer is separated and washed twice by water and once by brine, dried over Na₂SO₄ and concentrated. The residue is purified by silica (EtOAc-hexanes 0-10%) to give the title compound as an oil (5.6 g). LCMS (m/z) M+Na=465.6; t_{R}=4.51 min with the long method.

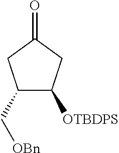

### (3S,4R)-3-(benzyloxymethyl)-4-(tert-butyldiphenylsilyloxy)cyclopentanone

((1R,2S)-2-(benzyloxymethyl)cyclopent-3-enyloxy)(tert-butyl)diphenylsilane (5.6 g) is dissolved in THF (50 mL) and a 9-BBN solution (50 mL, 0.5 M in THF) is added dropwise at 0° C. The mixture is stirred at rt overnight (20 hr), then again is cooled to 0° C. This is added a NaOH solution (12 mL, 3 M) dropwise followed by hydroperoxide (12 mL, 30% in water). The resulting mixture is then stirred at rt for 3 hr. The reaction mixture is added water and extracted by ethyl acetate, the EtOAc layer is separated and washed with water and brine, dried, separated and concentrated. The residue is used in next step directly. LCMS (m/z) M+H=461.6; t_{R}=3.97 min.

Oxalyl chloride (2.16 mL) is dissolved in THF (30 mL) at −78° C. To this is added DMSO (4 mL) dropwise and the resulting mixture is stirred for 30 min. A solution of the crude alcohol (˜6 g) from last step in THF (30 mL) is added dropwise and the mixture is stirred for 2 hr. Triethylamine (8 mL) is added and the resulting reaction mixture is stirred at rt for 30 min. The mixture is added water (100 mL), extracted with ethyl acetate (100 mL). EtOAc layers are separated, washed twice with water and once with brine, dried, concentrated and the residue is purified on silica (EtOAc-hexanes 0-10%) to give the title compound (2.3 g). LCMS (m/z) M+Na=481.4; t_{R}=3.87 min; ¹H NMR (500 MHz, CDCl₃) δ 7.64 (d, J=6.8 Hz, 2H), 7.61 (dd, J=6.8 Hz, 2H), 7.46-7.40 (m, 2H), 7.38-7.33 (m, 4H), 7.32-7.24 (m, 3H), 7.17 (d, J=6.9 Hz, 2H), 4.43-4.37 (m, 1H), 4.36 (d, J=12.6 Hz, 1H), 4.31 (d, J=12.6 Hz, 1H), 3.33 (dd, J=9.4, 4.9 Hz, 1H), 3.30 (dd, J=9.4, 5.2 Hz, 1H), 2.58 (dd, J=18.3, 8.2 Hz, 1H), 2.53-2.47 (m, 1H), 2.34 (dd, J=18.4, 5.9 Hz, 1H), 2.20 (dd, J=18.4, 4.1 Hz, 1H), 2.10 (dd, J=18.3, 5.6 Hz, 1H), 1.05 (s, 9H).

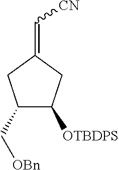

### 2-((3S,4R)-3-(benzyloxymethyl)-4-(tert-butyldiphenylsilyloxy)cyclopentylidene)acetonitrile

To a stirred solution of anhydrous acetonitrile (0.3 mL) in THF (3 mL) at −78° C. is added dropwise butyl lithium (1.6 mL, 2.5 M in THF) and the resulting mixture is stirred for 10 min. A solution of (3S,4R)-3-(benzyloxymethyl)-4-(tert-butyldiphenylsilyloxy)cyclopentanone (0.916 g) in THF (2 mL) is added dropwise, and the resulting mixture is stirred for 50 min at −78° C. TLC shows most of the ketone disappears. Saturated ammonium chloride aqueous solution (5 mL) and EtOAc (20 mL) are added. The organic layer is separated, washed with water and brine, dried, concentrated and the residue is purified on silica (EtOAc-hexanes 0-20%) to give 2-((3S,4R)-3-(benzyloxymethyl)-1,4-dihydroxycyclopentyl)acetonitrile (0.88 g) as an oil. LCMS (m/z) M+H=262.3; t_{R}=3.95 min.

The product (0.88 g) is dissolved in pyridine (10 mL) at 0° C. and thionyl chloride (1 mL) is added dropwise. The mixture is stirred at rt for 2 hr, concentrated and the residue dissolves in EtOAc (20 mL). The mixtue is washed twice with 2 N HCl, once with NaHCO₃ solution and once with brine, dried, concentrated and the residue is purified on silica (EtOAc-hexanes 0-10%) to give the title compound (0.5 g) as an oil.

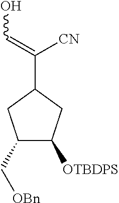

### 2-((3S,4R)-3-(benzyloxymethyl)-4-(tert-butyldiphenylsilyloxy)cyclopentyl)-3-hydroxyacrylonitrile

A mixture of 10% Pd/C (50 mg) and 2-((3S,4R)-3-(benzyloxymethyl)-4-(tert-butyldiphenylsilyloxy)cyclopentylidene)acetonitrile (0.5 g) in methanol (3 mL) is stirred under H₂ for 3 hr until TLC shows no starting material is left. The mixture is filtered and the filtrate concentrated to give 2-((3S,4R)-3-(benzyloxymethyl)-4-(tert-butyldiphenylsilyloxy)-cyclopentyl)acetonitrile as an oil (0.5 g). LCMS (m/z) M+Na=506.5; t_{R}=4.19 min.

A mixture of that product (1.1 g) and 1-tert-butoxy-N,N,N′,N′-tetramethylmethanediamine (3 mL) in DMF (6 mL) is stirred at 95° C. for 20 hr. The mixture is quenched with water and added EtOAc. EtOAc layer is seperated and washed with water and brine, and concentrated. The residue is dissolved in chloroform (25 mL), water (12 mL) and TFA (0.4 mL) is added and the mixture is stirred at rt for 2 hr. Chloroform layer is separated and washed with water twice, dried, concentrated, the residue is purified on silica (EtOAc-hexanes 0-30%) to give the title compound as an oil (0.58 g). LCMS (m/z) M+CH₃OH+H=544.5; t_{R}=3.95 min.

### Example 26

### Preparation of ((1S,2S,4R)-4-(4-((S)-2,3-dihydro-1H-inden-1-ylamino)furo[3,2-d]pyrimidin-7-yl)-2-hydroxycyclopentyl)methyl sulfamate (Compound 17)

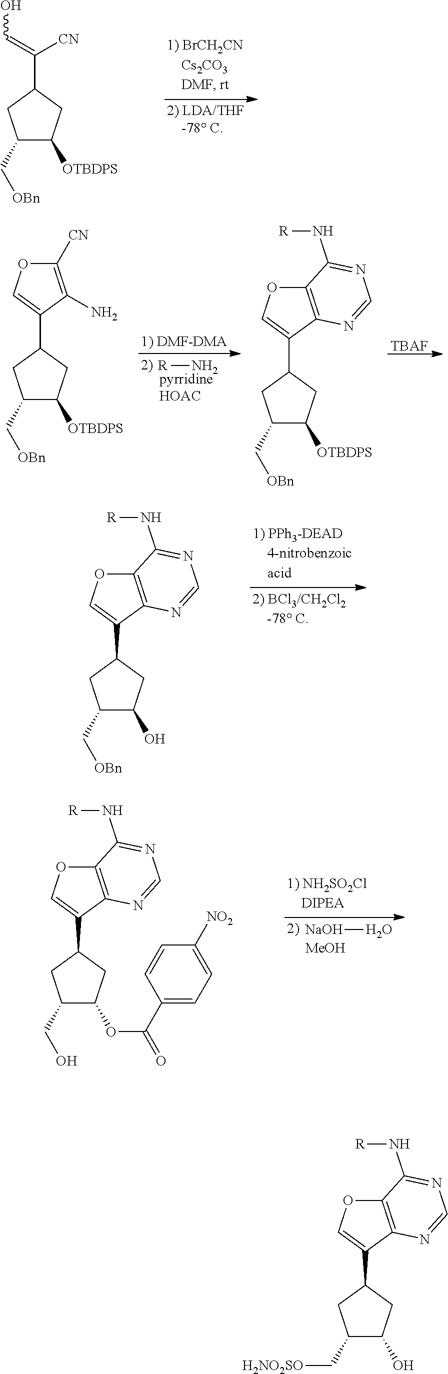

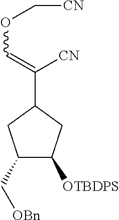

### 2-((3S,4R)-3-(benzyloxymethyl)-4-(tert-butyldiphenylsilyloxy)cyclopentyl)-3-(cyanomethoxy)acrylonitrile

2-((3S,4R)-3-(benzyloxymethyl)-4-(tert-butyldiphenylsilyloxy)cyclopentyl)-3-hydroxyacrylonitrile (0.59 g), bromoacetanitrile (0.2 g) and Cs₂CO₃ (0.567 g) are added to DMF (5 mL) and the mixture is stirred at room temperature for 2 hr. The mixture is quenched with water and extracted with EtOAc. The organic layer is separated, washed with water and brine, dried, concentrated. The residue is purified on silica (0-30% EtOAc-hexanes) to give 0.45 g of the tile compound. LCMS (m/z) M+Na=573.5; t_{R}=4.14 min; ¹H NMR (500 MHz, DMSO-d₆) δ 7.62-7.55 (m, 4H), 7.48-7.36 (m, 6H), 7.35-7.24 (m, 4H), 7.22-7.14 (m, 2H), 5.09 (s, 0.8H), 5.02 (s, 1.2H), 4.22 (s, 0.8H), 4.32-4.25 (m, 3.2H), 4.20-4.14 (m, 0.4H), 4.07-4.00 (m, 0.6H), 3.22-3.06 (m, 1.6H), 2.89-2.81 (m, 0.4H), 2.30-2.17 (m, 0.8H), 2.10-2.03 (m, 0.4H), 1.96-1.89 (m, 0.6H), 1.83-1.73 (m, 0.6H), 1.70-1.62 (m, 0.8H), 1.55-1.45 (m, 0.4H), 1.43-1.37 (m, 0.4H), 1.01 (s, 9H).

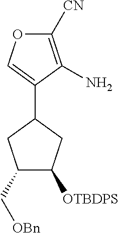

### 3-Amino-4-((3S,4R)-3-(benzyloxymethyl)-4-(tert-butyldiphenylsilyloxy)cyclopentyl)furan-2-carbonitrile

2-((3S,4R)-3-(benzyloxymethyl)-4-(tert-butyldiphenylsilyloxy)cyclopentyl)-3-(cyanomethoxy)acrylonitrile (0.45 g) is dissolved in THF (5 mL) and the solution is added dropwise lithium diisopropylamide (0.8 mL, 2 M in THF) at −78° C. The mixture is stirred for 30 min and added water. The mixture is extracted with EtOAc, the organic layer is separated, washed with water and brine, dried and concentrated. The residue is purified on silica (0-20%, EtOAc-hexanes) to give the tilte compound (50 mg). LCMS (m/z) M+H=551.6; t_{R}=4.22 min.

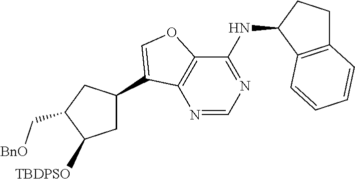

### 7-((1R,3S,4R)-3-(benzyloxymethyl)-4-(tert-butyldiphenylsilyloxy)cyclopentyl)-N—((S)-2,3-dihydro-1H-inden-1-yl)furo[3,2-d]pyrimidin-4-amine

A mixture of dimethylformamide dimethyl acetal (0.3 ml) and 3-amino-4-((3S,4R)-3-(benzyloxymethyl)-4-(tert-butyldiphenylsilyloxy)cyclopentyl)furan-2-carbonitrile (57 mg) in toluene (3 mL) is heated to 110° C. for 2 hours. The mixture is concentrated to give N′-(4-((3S,4R)-3-(benzyloxymethyl)-4-(tert-butyldiphenylsilyloxy)cyclopentyl)-2-cyanofuran-3-yl)-N,N-dimethylformimidamide as a residue: LCMS (m/z) M+H=606.7; t_{R}=4.54 min with the long method. A stirred mixture of the residue (60 mg), (S)-1-aminoindane (70 mg), pyridine (240 mg) and acetic acid (180 mg) in acetonitrile (3 mL) is heated to 110° C. with microwave for 90 min. The reaction mixture is concentrated, added water, extracted with EtOAc. The organic layer is separated, washed with water and brine, dried, concentrated and the residue purified on TLC (hexane-EtOAc 5:1) to give the title compound (16 mg). LCMS (m/z) M+H=694.7; t_{R}=4.56 min with the long method.

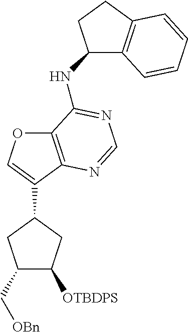

### 7-((1S,3S,4R)-3-(benzyloxymethyl)-4-(tert-butyldiphenylsilyloxy)cyclopentyl)-N-((S)-2,3-dihydro-1H-inden-1-yl)furo[3,2-c]pyrimidin-4-amine

The title compound (18 mg) is also isolated as the diastereoisomer from the last procedure. LCMS (m/z) M+H=694.7; t_{R}=4.42 min with the long method.

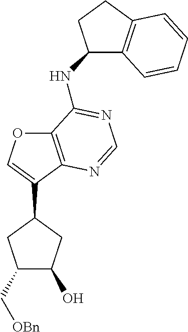

### (1R,2S,4R)-2-(benzyloxymethyl)-4-(4-((S)-2,3-dihydro-1H-inden-1-ylamino)furo[3,2-d]pyrimidin-7-yl)cyclopentanol

7-((1R,3S,4R)-3-(benzyloxymethyl)-4-(tert-butyldiphenylsilyloxy)cyclopentyl)-N—((S)-2,3-dihydro-1H-inden-1-yl)furo[3,2-d]pyrimidin-4-amine (30 mg) and tetrabutylammonium fluoride (0.5 mL, 1 M in THF) is added to THF (0.5 ml) and the mixture is stirred at rt for 6 hours. The mixture is purified on TLC (hexane-EtOAc 2:1) to give the title compound (15 mg). LCMS (m/z) M+H=456.5; t_{R}=2.40 min.

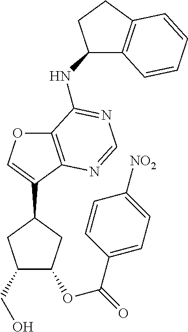

### (1S,2S,4R)-4-(4-((S)-2,3-dihydro-1H-inden-1-ylamino)furo[3,2-d]pyrimidin-7-yl)-2-(hydroxymethyl)cyclopentyl 4-nitrobenzoate

A mixture of (1R,2S,4R)-2-(benzyloxymethyl)-4-(4-((S)-2,3-dihydro-1H-inden-1-ylamino)furo[3,2-c]pyrimidin-7-yl)cyclopentanol (15 mg), 4-nitrobenzoic acide (30 mg) and triphenyl phosphine (20 mg) in THF (2 mL) is added diethyl azodicarboxylate (0.06 mL, 40% in toluene) and the mixture is stirred at rt overnight. The mixture is then purified on TLC (hexanes-EtOAc 2:1) to give the product (1S,2S,4R)-2-(benzyloxymethyl)-4-(4-((S)-2,3-dihydro-1H-inden-1-ylamino)furo[3,2-d]pyrimidin-7-yl)cyclopentyl 4-nitrobenzoate (14 mg). LCMS (m/z) M+H=605.6; t_{R}=3.22 min.

A solution of that product (14 mg) in dichloromethane (1 ml) at −78° C. is added borane trichloride (0.1 ml) dropwise. The resulting mixture is stirred for 2 hours, concentrated and purified on TLC (dichloromethane-methanol 20:1) to give the title compound (8 mg). LCMS (m/z) M+H=515.6; t_{R}=2.57 min.

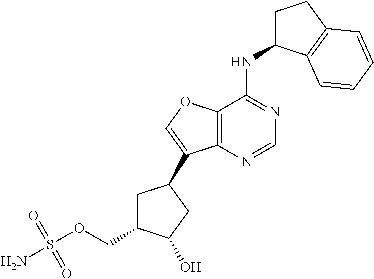

### ((1S,2S,4R)-4-(4-((S)-2,3-dihydro-1H-inden-1-ylamino)furo[3,2-d]pyrimidin-7-yl)-2-hydroxycyclopentyl)methyl sulfamate (Compound 17)

A mixture of (1S,2S,4R)-4-(4-((S)-2,3-dihydro-1H-inden-1-ylamino)furo[3,2-d]pyrimidin-7-yl)-2-(hydroxymethyl)cyclopentyl 4-nitrobenzoate (4 mg) and N,N-diisopropylethylamine (0.1 mL) in DMF (1 mL) at 0° C. is added sulfamoyl chloride (0.1 mL, 1 M in acetonitrile) dropwise. The mixture is stirred at rt for 4 hours, quenched with water and extracted with EtOAc. The organic layer is separated, washed with water and brine, dried, concentrated to afford (1S,2S,4R)-4-(4-((S)-2,3-dihydro-1H-inden-1-ylamino)furo[3,2-d]pyrimidin-7-yl)-2-(sulfamoyloxymethyl)cyclopentyl 4-nitrobenzoate as a residue. LCMS (m/z) M+H=594.6; t_{R}=2.74 min.

The residue is dissolved in methanol (2 ml) and LiOH is added, and the mixture is stirred at rt for 1 hour. AcOH is added to the mixture to adjust pH to 6-7. The mixture is concentrated and residue purified on TLC (dichloromethane-methanol 20:1) to give the title compound (1.1 mg). LCMS (m/z) M+H=445.4; t_{R}=1.72 min.

### Example 27

### Preparation of [(1S,2S,4R)-4-[4-(cyclohexylmethylamino)thieno[3,2-d]pyrimidin-7-yl]-2-hydroxy-cyclopentyl]methyl sulfamate (Compound 18)

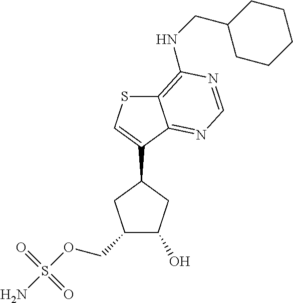

### 1. 3-(4,4,5,5-tetramethyl-1,3,2-dioxaborolan-2-yl)cyclopent-2-en-1-one

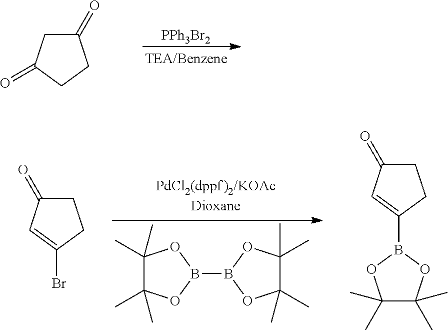

Cyclopentane-1,3-dione (5.28 g, 53.8 mmol) and triethylamine (8.25 mL, 59.2 mmol) are added to a stirred suspension of dibromo-triphenyl phosphine (25.08 g, 59.2 mmol) in benzene (100 mL) at room temperature. The resulting mixture is stirred at room temperature for 18 hr, then concentrated in vacuo. The resulting residue is filtered through a pad of silica gel and rinsed with ether. The ether is collected and removed in vacuo and the crude product is purified by chromatograph to give 3-bromocyclopent-2-en-1-one (8.10 g, 94%) as colorless oil.

PdCl₂(dppf)₂ (0.32 g, 0.44 mmol) is added to a degassed mixture of 3-bromocyclopent-2-en-1-one (1.00 g, 6.21 mmol), bis(pinacolato)diboron (1.73 g, 6.83 mmol), KOAc (1.22 g, 12.40 mmol) in 1,4-dioxane (15 mL) at room temperature. The resulting mixture is heated to 100° C. for 18 hr under nitrogen atmosphere. The reaction mixture is concentrated in vacuo and the crude product is purified by chromatograph to give 3-(4,4,5,5-tetramethyl-1,3,2-dioxaborolan-2-yl)cyclopent-2-en-1-one as a solid (1.00 g, 78%).

### 2. 3-(4-(tert-butylsulfonyl)thieno[3,2-d]ovrimidin-7-yl)-5-((methoxymethoxy)methyl)cyclopent-2-enone

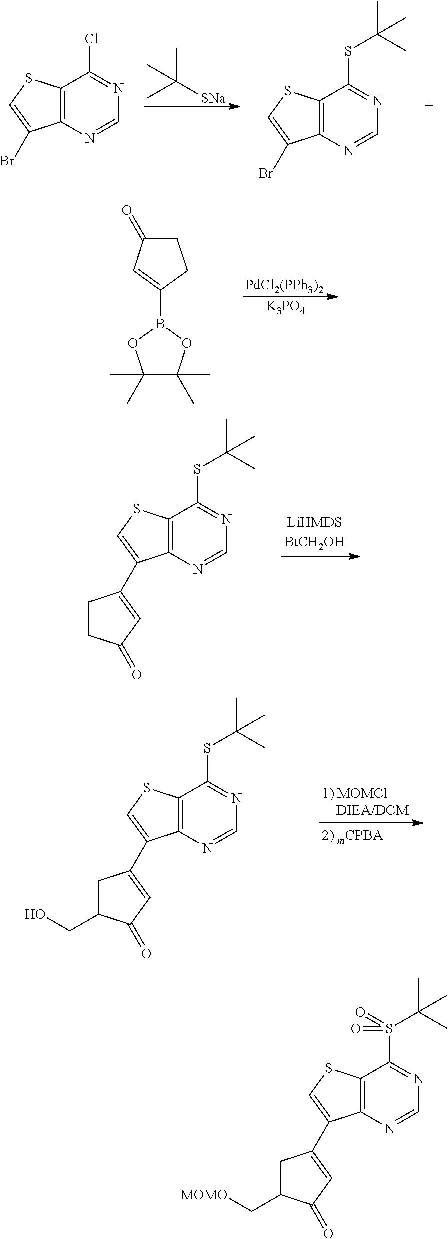

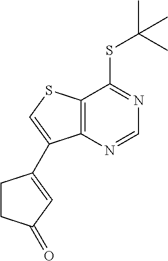

### 3-(4-tert-butylsulfanylthieno[3,2-d]pyrimidin-7-yl)cyclopent-2-en-1-one

Sodium 2-methyl-2-propanethiolate (5.60 g, 50 mmol) is added to a solution of 7-bromo-4-chloro-thieno[3,2-d]pyrimidine (10.0 g, 40 mmol) in DMSO (150 mL) at 0° C. The resulting mixture is warmed up to room temperature and stirred for 4 hr, poured into ice (1500 g) and stirred until all ice melt during which a precipitate formed. The precipitate is collected by filtration, washed with water (500 mL) and dried over lyophilization to give 7-bromo-4-tert-butylsulfanyl-thieno[3,2-d]pyrimidine (10.38 g, 86%). LCMS (m/z) M+H=303.2 and 305.1; t_{R}=3.51 min.

PdCl₂(PPh₃)₂ (1.65 g, 2.35 mmol) is added to a solution of 3-(4,4,5,5-tetramethyl-1,3,2-dioxaborolan-2-yl)cyclopent-2-en-1-one (8.10 g, 38.9 mmol), 7-bromo-4-tert-butylsulfanyl-thieno[3,2-d]pyrimidine (10.30 g, 33.9 mmol) and K₃PO₄ (21.52 g, 101 mmol) in a mixture of 1,4-dioxane (250 mL) and water (50 mL). The resulting mixture is heated to 80° C. for 12 hr and concentrated in vacuo. The crude residue is purified by chromatograph to give desired title product as a solid (7.65 g, 73%). LCMS (m/z) M+H=305.4; t_{R}=3.27 min.

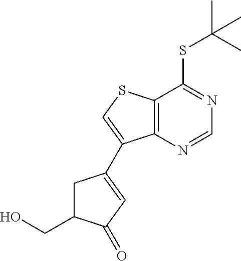

### 3. 3-(4-tert-butylsulfanvIthieno[3,2-d]pyrimidin-7-yl)-5-(hydroxymethyl)cyclopent-2-en-1-one

Lithium bis(trimethylsilyl)amide (1.0 M in THF, 0.66 mL, 0.66 mmol) is added to a solution of 3-(4-tert-butylsulfanylthieno[3,2-d]pyrimidin-7-yl)cyclopent-2-en-1-one (0.10 g, 0.33 mmol) in THF (2.0 mL) at −78° C. and the resulting mixture is stirred at this temperature over 1 hr. To this mixture, a solution of 1-hydroxymethylbenzotriazole (0.10 g, 0.66 mmol) in a mixture of THF (1.0 mL) and hexamethylphosphoramide (HMPA, 0.10 mL) is added. The resulting mixture is stirred at −78° C. for 2 hr then quenched with saturated aqueous NH₄Cl (1.0 mL). THF is removed in vacuo and the resulting residue is taken up in dichloromethane, dried with Na₂SO₄, filtered and concentrated in vacuo. The crude is purified by chromatograph to give desired title compound as a solid (0.08 g, 69%). LCMS (m/z) M+H=335.4; t_{R}=2.89 min.

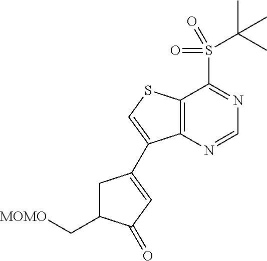

### 3-(4-tert-butylsulfonylthieno[3,2-d]pyrimidin-7-yl)-5-(methoxymethoxymethyl)cyclopent-2-en-1-one

Methyl chloromethyl ether (0.17 mL, 2.23 mmol) is added to a mixture of 3-(4-tert-butylsulfanylthieno[3,2-d]pyrimidin-7-yl)-5-(hydroxymethyl)cyclopent-2-en-1-one (0.18 g, 1.49 mmol) and N,N-diisopropylethylamine (0.78 mL, 4.47 mmol) in dichloromethane (20 mL) at 0° C. The resulting mixture is stirred at room temperature for 16 hr, concentrated in vacuo, the crude residue is purified by chromatograph to afford 3-(4-tert-butylsulfanylthieno[3,2-d]pyrimidin-7-yl)-5-(methoxymethoxymethyl)cyclopent-2-en-1-one as a solid (0.33 g, 58%). LCMS (m/z) M+H=379.4; t_{R}=3.37 min.

m-Chloroperoxybenzoic acid (2.66 g, 11.87 mmol) is added to a solution of 3-(4-tert-butylsulfanylthieno[3,2-d]pyrimidin-7-yl)-5-(methoxymethoxymethyl)cyclopent-2-en-1-one (2.00 g, 5.28 mmol) in dichloromethane (50 mL) at 0° C. The resulting mixture is stirred at room temperature for 16 hr and concentrated in vacuo. The crude residue is purified by chromatograph to give the title compound (1.82 g, 84%). LCMS (m/z) M+H=411.4; t_{R}=2.52 min.

### 4. (1S,5R)-3-(4-ted-butylsulfonylthieno[3,2-d]oyrimidin-7-yl)-5-(methoxymethoxymethyl)cyclopent-2-en-1-ol and (1S,5S)-3-(4-tert-butylsulfonylthieno[3,2-d]pyrimidin-7-yl)-5-(methoxymethoxymethyl)cyclopent-2-en-1-ol

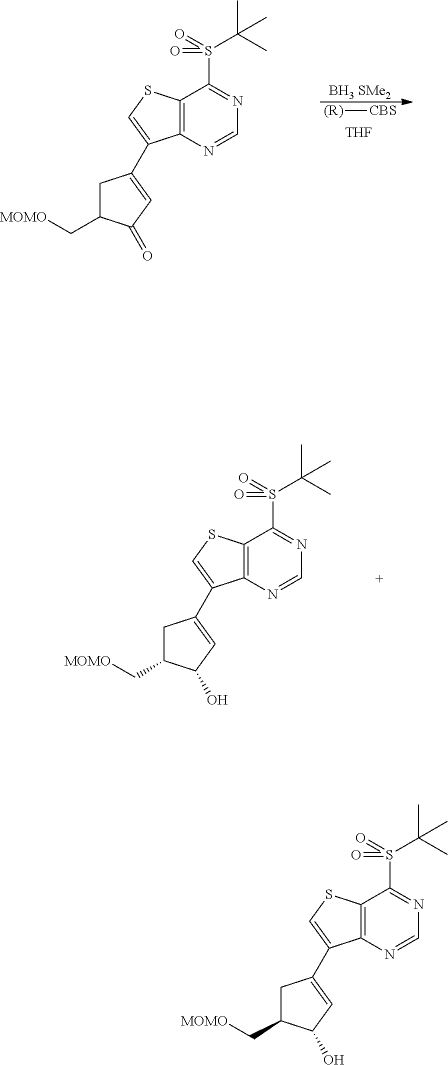

Borane dimethyl sulfide (0.28 mL, 2.91 mmol) is added to a solution of 3-(4-ted-butyl sulfonylthieno[3,2-d]pyrimidin-7-yl)-5-(methoxymethoxymethyl)cyclopent-2-en-1-one (1.0 g, 2.43 mmol) and (R)-CBS (1 M in THF, 0.24 mL, 0.24 mmol) in anhydrous THF (20 mL) at 0° C. The resulting mixture is stirred at 0° C. for 1 hr and quenched with saturated aqueous NH₄Cl followed by extraction with EtOAc. The combined organic layer is separated, dried with Na₂SO₄, filtered and concentrated in vacuo. The crude is purified by chromatograph to give (1S,5R)-3-(4-tert-butylsulfonylth ieno[3,2-d]pyrimidin-7-yl)-5-(methoxymethoxymethyl)cyclopent-2-en-1-ol (0.45 g, 45% yield), LCMS (m/z) M+H=435.4; t_{R}=2.40 min and (1S,5S)-3-(4-tert-butylsulfonylthieno[3,2-d]pyrimidin-7-yl)-5-(methoxymethoxymethyl)cyclopent-2-en-1-ol (0.48 g, 48%), LCMS (m/z) M+H=435.4; t_{R}=2.46 min.

### 5. tert-butyl-[(1S,2S)-(4S)-4-(4-tert-butylsulfonylthieno[3,2-d]oyrimidin-7-yl)-2-(methoxymethoxymethyl)cyclopentoxy]-dimethyl-silane and ted-butyl-[(1S,2S)-(4R)-4-(4-ted-butylsulfonylthieno[3,2-d]oyrimidin-7-yl)-2-(methoxymethoxymethyl)cyclopentoxy]-dimethyl-silane

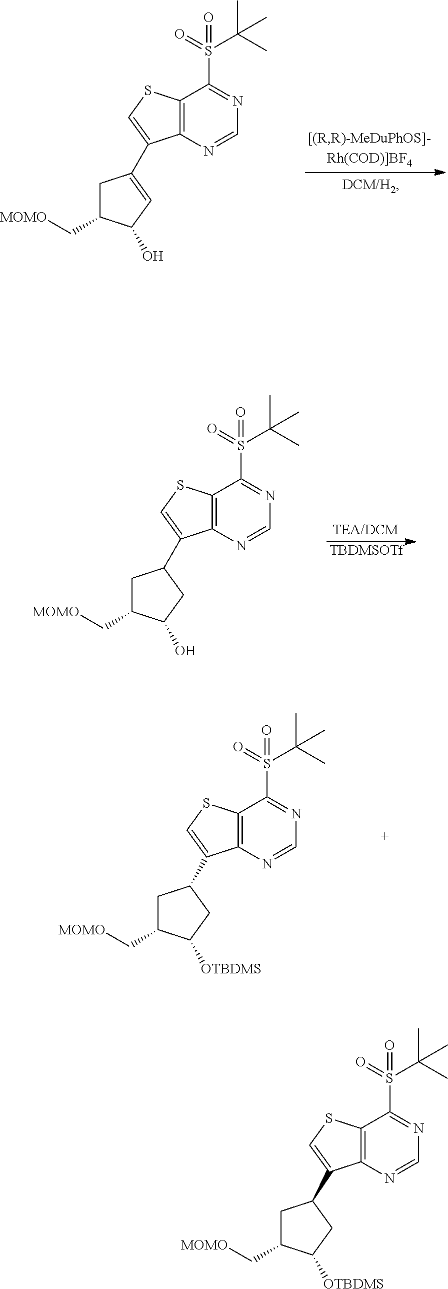

To a stirred solution of (1S,5S)-3-(4-tert-butylsulfonylthieno[3,2-d]pyrimidin-7-yl)-5-(methoxymethoxymethyl)cyclopent-2-en-1-ol (0.05 g, 0.12 mmol) in dichloromethane (10 mL) is added {[(R,R)-MeDuPhos]Rh(COD)}BF₄ (0.007 g, 0.012 mmol). The resulting mixture is stirred under hydrogen balloon for 16 hr and concentrated in vacuo. The crude reside is purified by chromatograph to give (1S,2S)-4-(4-tert-butylsulfonylthieno[3,2-d]pyrimidin-7-yl)-2-(methoxymethoxymethyl)cyclopentanol (0.039 g, 78%) containing two stereoiosmers that are not separable and is used as a mixture for next step. LCMS (m/z) M+H=415.4.

To the mixture of (1S,2S)-4-(4-tert-butylsulfonylthieno[3,2-d]pyrimidin-7-yl)-2-(methoxymethoxymethyl)cyclopentanol (0.078 g, 0.19 mmol) in anhydrous dichloromethane (10 mL) is added tert-butyldimethylsilyltrifluoromethanesulfonate (0.054 mL, 0.24 mmol) and triethylamine (0.079 mL, 0.56 mmol) at 0° C. The resulting mixture is stirred at room temperature for 2 hr and concentrated in vacuo. The crude residue is purified by chromatograph to give tert-butyl-[(1S,2S)-(4S)-4-(4-tert-butylsulfonylthieno[3,2-d]pyrimidin-7-yl)-2-(methoxymethoxymethyl)cyclopentoxy]-dimethyl-silane (0.022 g, 22%), LCMS (m/z) M+H=529.5; t_{R}=4.14 min and tert-butyl-[(1S,2S)-(4R)-4-(4-tert-butylsulfonylth ieno[3,2-d]pyrimidin-7-yl)-2-(methoxymethoxymethyl)cyclopentoxy]-dimethyl-silane (0.073 g, 74%), LCMS (m/z) M+H=529.5; t_{R}=4.27 min.

### 6. [(1S,2S,4R)-4-[4-(cyclohexylmethylamino)thieno[3,2-d]pyrimidin-7-yl]-2-hydroxy-cyclopentyl]methyl sulfamate (Compound 18)

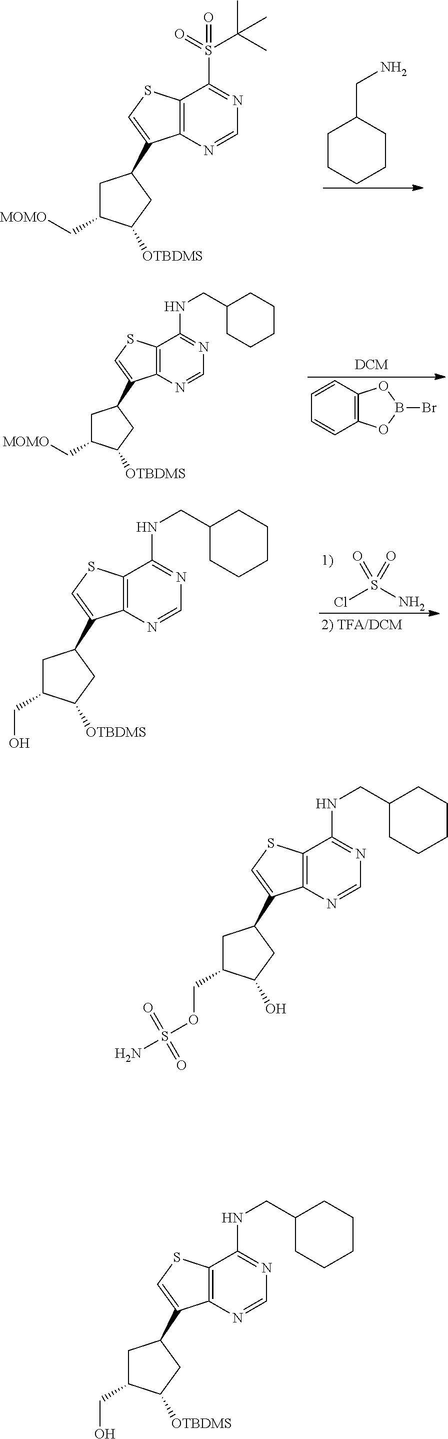

### [(1S,2S,4R)-2-[tert-butyl(dimethyl)silyl]oxy-4-[4-(cyclohexylmethylamino)thieno[3,2-d]pyrimidin-7-yl]cyclopentyl]methanol

A mixture of cyclohexanemethylamine (0.12 mL, 0.95 mmol), tert-butyl-[(1S,2S)-(4R)-4-(4-tert-butylsulfonylthieno[3,2-d]pyrimidin-7-yl)-2-(methoxymethoxymethyl)cyclopentoxy]-dimethyl-silane (0.05 g, 0.095 mmol) in dimethoxyethane (2 mL) is heated to 150° C. by microwave for 3 hr. The reaction mixture is concentrated in vacuo and the crude residue is purified by chromatograph to give 7-[(1R,3S,4S)-3-[tert-butyl(dimethyl)silyl]oxy-4-(methoxymethoxymethyl)cyclopentyl]-N-(cyclohexylmethyl)thieno[3,2-d]pyrimidin-4-amine (0.049 g, 100%). LCMS (m/z) M+H=520.7; t_{R}=3.95 min.

To a solution of this compound (0.049 g, 0.094 mmol) in dichloromethane (10 mL) is added a solution of 2-bromo-1,3,2-benzodioxaborole (0.093 g, 0.47 mmol) in dichloromethane (1 mL) at −78° C. and the mixture is stirred at the same temperature for 1 hr. The reaction mixture is quenched with a mixture of THF (3 mL) and saturated aqueous NaHCO₃ (3 mL) followed by extraction with EtOAc. The EtOAc portion is separated, dried with Na₂SO₄, filtered and concentrated in vauco. The crude residue is purified by chromatograph to give the desired [(1S,2S,4R)-2-[tert-butyl(dimethyl)silyl]oxy-4-[4-(cyclohexylmethylamino)thieno[3,2-d]pyrimidin-7-yl]cyclopentyl]methanol as a solid (0.035 g, 80%). LCMS (m/z) M+H=476.6; t_{R}=3.31 min.

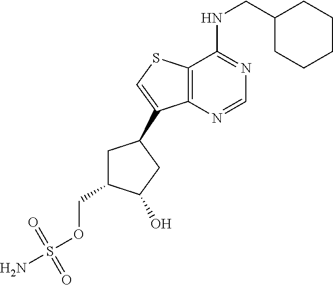

### [(1S,2S,4R)-4-[4-(cyclohexylmethylamino)thieno[3,2-d]pyrimidin-7-yl]-2-hydroxy-cyclopentyl]methyl sulfamate (Compound 18)

Sulfamoyl chloride (2.0 M in CH₃CN) (0.15 ml, 0.29 mmol) is added to a mixture of [(1S,2S,4R)-2-[tert-butyl(dimethyl)silyl]oxy-4-[4-(cyclohexylmethylamino)thieno[3,2-d]pyrimidin-7-yl]cyclopentyl]methanol (0.035 g, 0.074 mmol) and triethylamine (0.10 mL, 0.74 mmol) in CH₃CN (5 mL) at room temperature and the resulting mixture is stirred at room temperature for 6 hr. The reaction mixture is quenched with MeOH and concentrated in vacuo. The crude residue is purified by preparative TLC to give desired [(1S,2S,4R)-2-[tert-butyl(dimethyl)silyl]oxy-4-[4-(cyclohexylmethylamino)thieno[3,2-d]pyrimidin-7-yl]cyclopentyl]methyl sulfamate (10 mg, 59%). LCMS (m/z) M+H=555.5; t_{R}=3.29 min.

A mixture of [(1S,2S,4R)-2-[tert-butyl(dimethyl)silyl]oxy-4-[4-(cyclohexylmethylamino)thieno[3,2-d]pyrimidin-7-yl]cyclopentyl]methyl sulfamate (0.01 g, 0.02 mmol) in a solution of TFA (70%) in dichloromethane (5.0 mL) is stirred at room temperature for 3 hr. The reaction mixture is concentrated in vacuo, the resulting crude is purified by preparative TLC to afford [(1S,2S,4R)-4-[4-(cyclohexylmethylamino)thieno[3,2-d]pyrimidin-7-yl]-2-hydroxy-cyclopentyl]methyl sulfamate (Compound 18) (7 mg, 68%). LCMS (m/z) M+H=441.4; t_{R}=2.17 min.

### Example 28

The following compounds are prepared essentially according to the procedures set forth above in Examples 1-27.

| | | LCMS |
|---|---|---|
| Compd. | | (m/z) |
| No. | Structure Chemical Name | M + H |
| 19 | 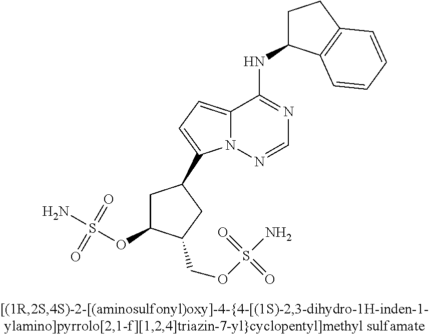 | 523.4 |
| 20 | 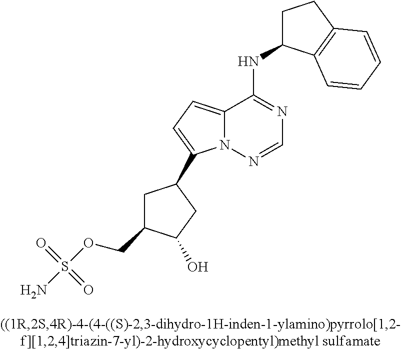 | 444.5 |
| 21 | 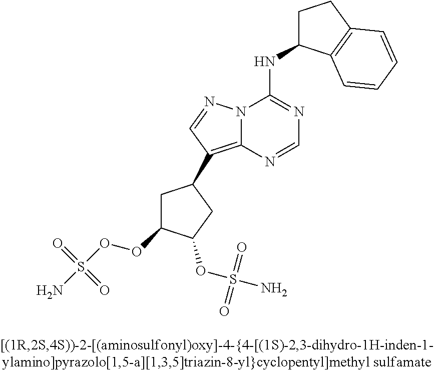 | 524.3 |
| 22 | 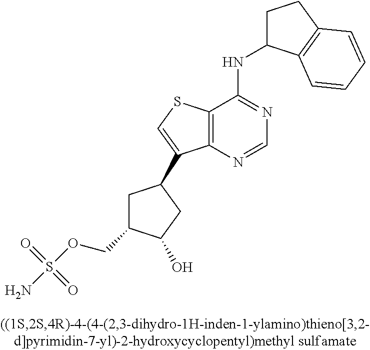 | 461.4 |
| 23 | 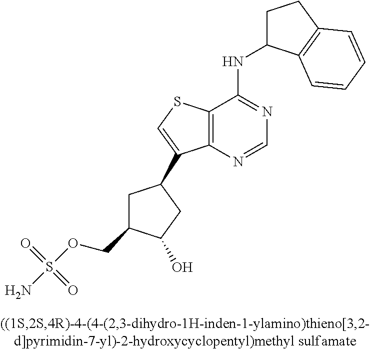 | 461.4 |
| 24 | 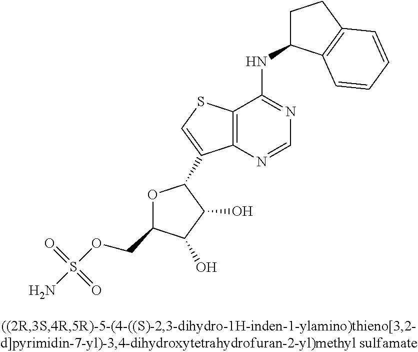 | 479.3 |
| 25 | 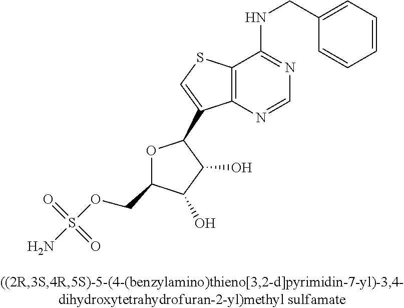 | 453.3 |
| 26 | 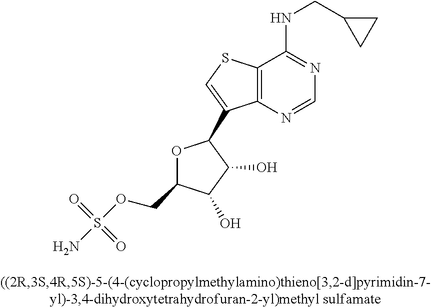 | 417.3 |
| 27 | 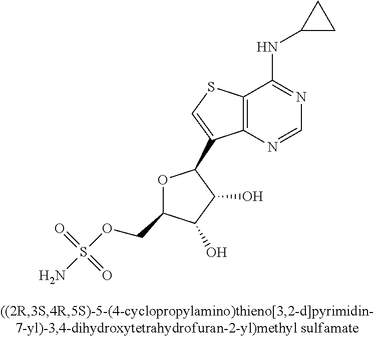 | 403.3 |
| 28 | 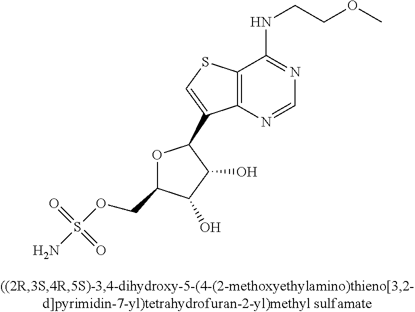 | 421.3 |
| 29 | 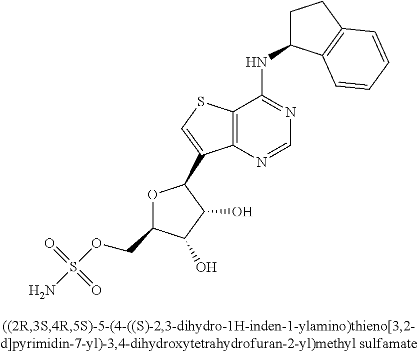 | 479.3 |
| 30 | 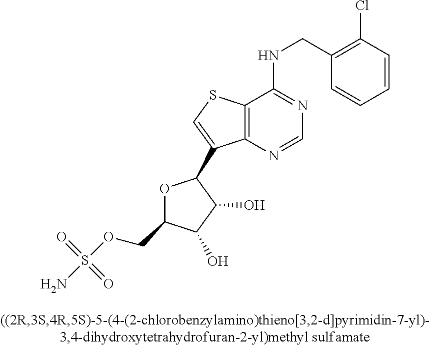 | 487.3 |
| 31 | 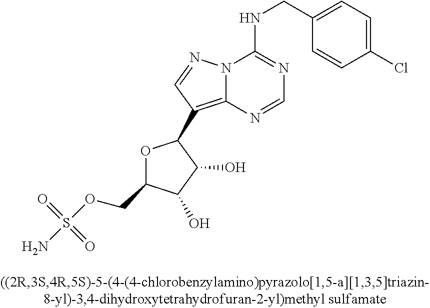 | 471.3 |
| 32 | 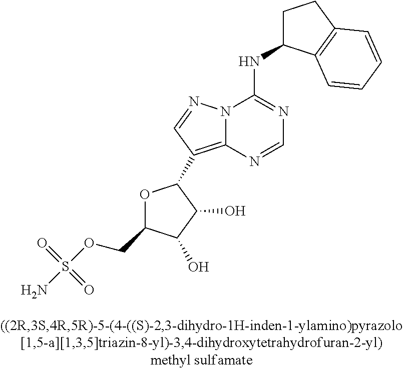 | 463.4 |
| 33 | 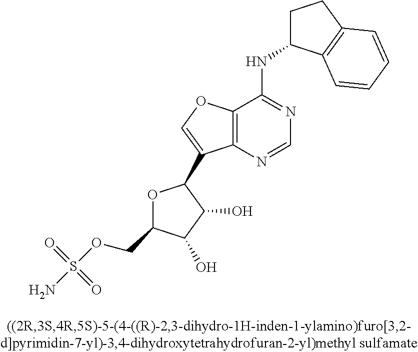 | 463.4 |
| 34 | 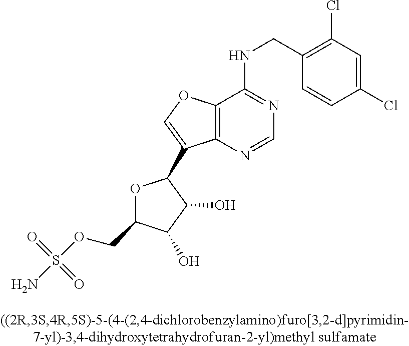 | 505.3 |
| 35 | 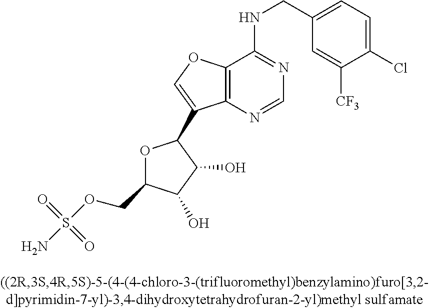 | 539.3 |
| 36 | 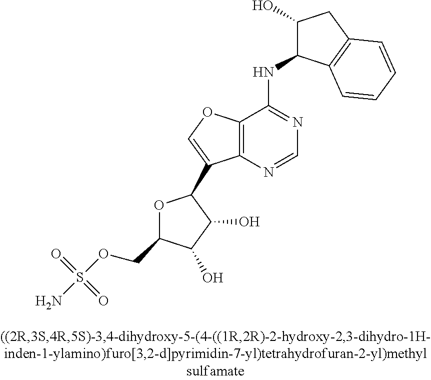 | 479.3 |
| 37 | 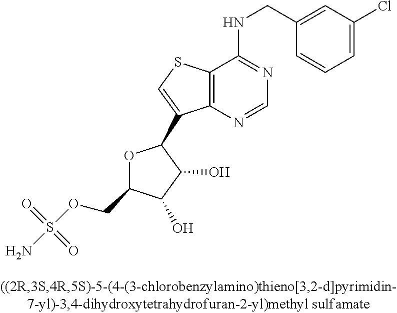 | 487.3 |
| 38 | 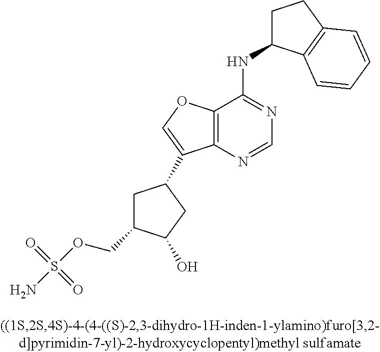 | 445.4 |
| 39 | 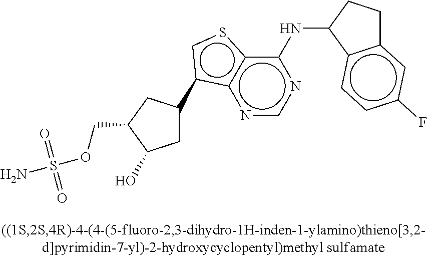 | 479.4 |
| 40 | 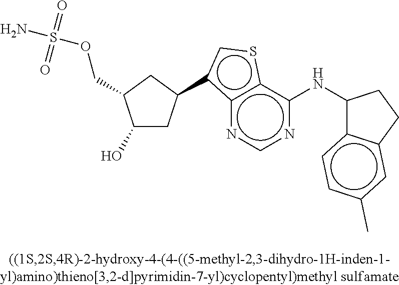 | 475.4 |
| 41 | 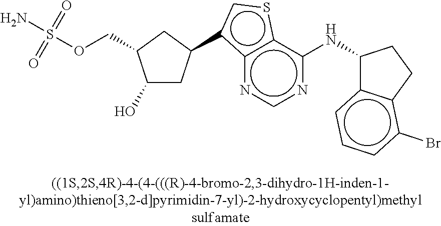 | 539.3 |
| 42 | 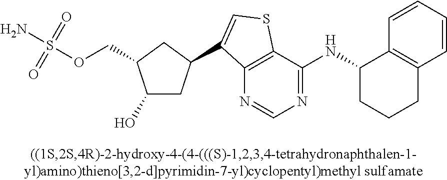 | 475.4 |
| 43 | 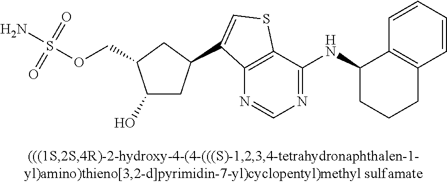 | 475.4 |
| 44 | 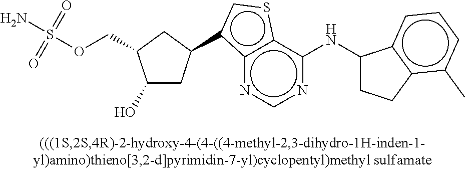 | 475.4 |
| 45 | 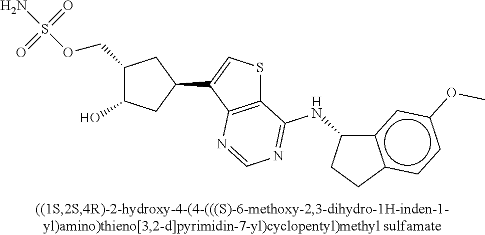 | 491.4 |
| 46 | 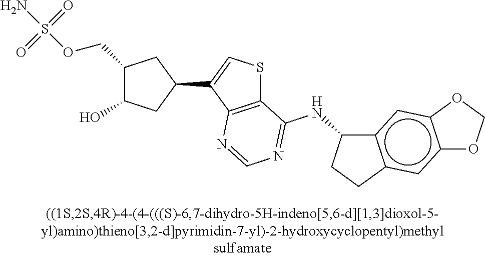 | 505.4 |
| 47 | 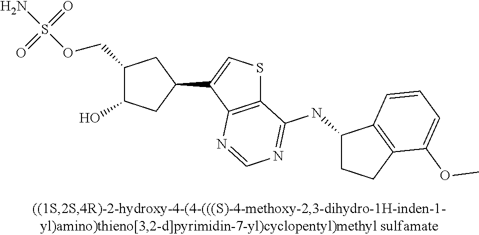 | 491.4 |
| 48 | 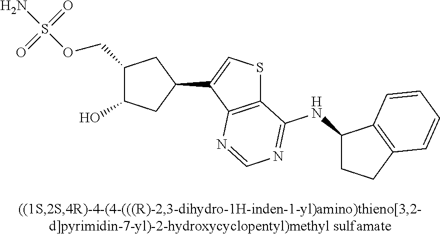 | 461.4 |
| 49 | 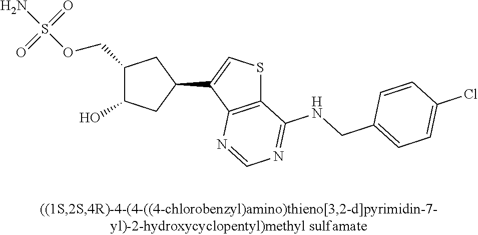 | 469.3 |
| 50 | 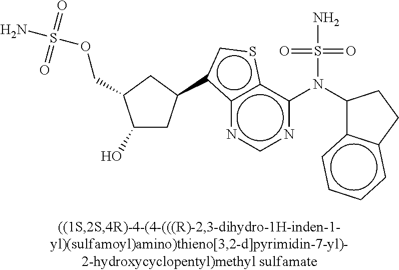 | 540.4 |
| 51 | 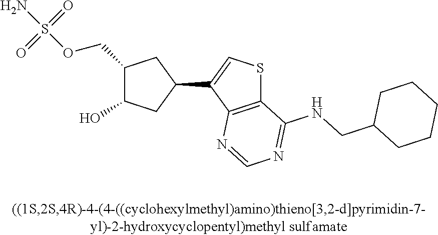 | 441.4 |
| 52 | 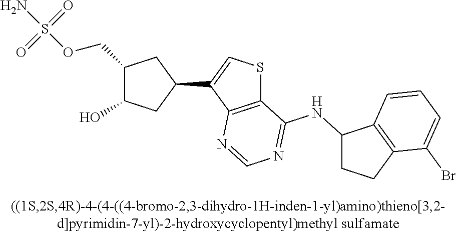 | 539.3 |
| 53 | 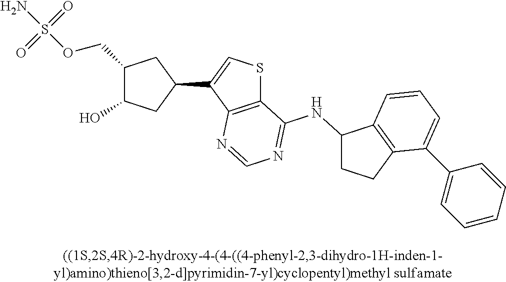 | 537.4 |
| 54 | 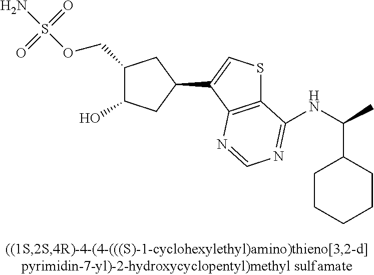 | 537.4 |
| 55 | 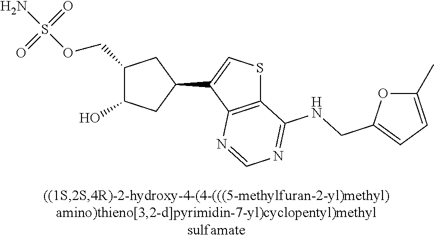 | 439.4 |
| 56 | 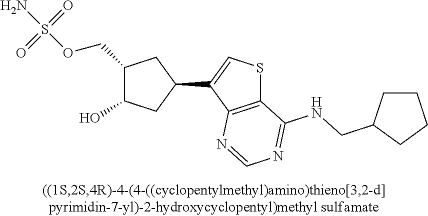 | 427.4 |
| 57 | 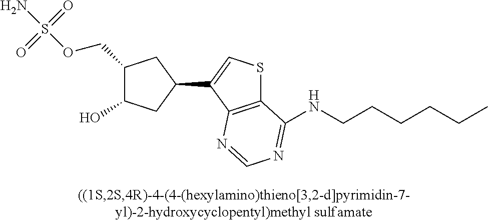 | 429.4 |
| 58 | 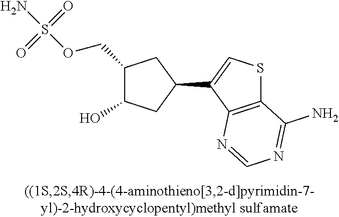 | 345.3 |

### Biological Evaluation

### Example 29

### Cell Proliferation Assays

A panel of cancer cell lines is obtained from ATCC (Rockville, Md.). Cell cultures are maintained in Hyclone DMEM/F12 medium (Logan, Utah) supplemented with 10% fetal bovine serum and 15 mM HEPES buffer, final pH 7.2, at 37° C. with a 5% CO₂ atmosphere. Cultures are maintained at sub-confluent densities.

For proliferation assays, cells are seeded into 96 well plates at 1,000-5,000 cells per well, depending on the cell line, and are incubated overnight. The following day, test compound, DMSO solution (negative control), or Actinomycin D (positive control) is added to the appropriate wells as 10× concentrated stocks prepared in phosphate buffered saline. The cell plates are then incubated for an additional 2-6 days, depending on the cell line, to allow proliferation to occur. To measure cell growth in adherent cultures, DNA amounts are measured in each well using the CyQuant DNA determination kit (Invitrogen, Carlsbad, Calif.) following the recommended protocol. Measurement of cell growth in suspension cultures is done by use of Celltiter Reagent (Promega, Madison, Wis.) at the end of treatment, following the recommended protocol. The percentage of cell growth is determined by comparing the cell growth in the presence of test compounds to the cells treated with DMSO vehicle (control, 100% growth) and cells treated with Actinomycin D (10 mM, 0% growth).

Several exemplary compounds useful in the methods of the disclosure are listed below. The range of their inhibitory activity against HCT-116 cell proliferation is demonstrated, where +++ stands for an IC₅₀ value that is less than 0.5 μM, ++ between 0.5 and 5 μM, + between 5 and 50 μM.

| Compound No. | Activity |
|---|---|
| 4 | +++ |
| 19 | + |
| 1 | + |
| 26 | + |
| 6 | ++ |
| 11 | +++ |
| 3 | ++ |
| 10 | ++ |
| 25 | ++ |
| 28 | + |
| 13 | + |
| 34 | + |

### Example 30

### E1-Activating Enzyme Assays

A time-resolved fluorescence energy transfer assay format is used to measure the in vitro activity of NEDD8 Activating Enzyme (NAE). The enzymatic reaction is performed in a total volume of 50 μL, containing 50 mM HEPES, pH 7.5, 0.05% BSA, 5 mM MgCl₂, 100 μM ATP, 10 nM Ubc12-GST, 75 nM NEDD8-Flag and 0.5 nM recombinant human NAE enzyme. The reaction mixture is incubated with and without compound inhibitor at 24° C. for 30 min in a 96 half-well plate. The reaction is then terminated with 25 μL of stop/detection buffer (0.1 M HEPES, pH 7.5, 0.05% Tween20, 20 mM EDTA, 410 mM KF, 81.8 ng/mL Europium-Cryptate-labelled monoclonal Flag-M2-specific antibody (CisBio International) and 8.125 μg/mL PHYCOLINK allophycocyanin (XL-APC)-labelled GST-specific antibody (Prozyme)). After incubation for 2 hours at 24° C., the plate is read on a Tecan Infinite M1000 instrument using a time-resolved fluorescence method. A similar assay protocol is used to measure other E1 enzymes. For the Sumo Activating Enzyme (SAE) assay everything is conducted as described for NAE except that Ubc12-GST and NEDD8-Flag are replaced with 10 nM Ubc9-GST and 125 nM Sumo-Flag respectively, and recombinant human SAE (0.6 nM) replaced NAE. The Ubiquitin Activating Enzyme (UAE) assay is performed as described for NAE except Ubc12-GST, NEDD8-Flag, and NAE are replaced with 5 nM Ubc2-GST, 125 nM UBB-Flag, and 1.5 nM UAE respectively.

Compounds of the disclosure are inhibitors of NAE. Several exemplary compounds useful in the methods of the disclosure are listed below. The range of their inhibitory activity on NAE is demonstrated, where +++ stands for an IC₅₀ value that is less than 0.5 μM, ++ between 0.5 and 5 μM, + between 5 and 50 μM.

| Compound No. | Activity |
|---|---|
| 4 | +++ |
| 19 | + |
| 1 | + |
| 37 | ++ |
| 11 | ++ |
| 3 | ++ |
| 10 | ++ |
| 30 | ++ |
| 6 | +++ |
| 35 | ++ |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference in their entirety for all purposes.

What is claimed is:

1. A compound according to formula: 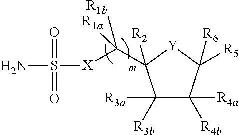
or a pharmaceutically acceptable salt thereof, wherein
m is 1, 2, or 3;
X is —CH₂—, —CH(halogen)-, —C(halogen)₂-, —O—, —NH—, or —N(C₁-C₆ alkyl)-;
Y is —O—, —S—, or —CR₁₀R₁₁—;
R₁ₐ and R_{1b} are each independently hydrogen, halogen, C₁-C₄ aliphatic, or C₁-C₄ haloaliphatic, or R₁ₐ and R_{1b}, taken together with the carbon to which they are attached, form a C₃-C₈ cycloalkyl ring, or one of R₁ₐ and R_{1b}, taken together with R₂ and the intervening carbon atoms, forms a C₃-C₈ cycloalkyl ring, or R₁ₐ and R_{1b} together form ═O;
R₂ is hydrogen, or C₁-C₄ aliphatic, or R₂ together with one R₁ₐ or R_{1b} and the carbon atoms to which they are attached forms C₃-C₈ cycloalkyl;
R₃ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —C(O)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of −OR₁₃, —N(R₁₄)(R₁₄), —CO₂R₁₃, and —C(O)N(R₁₄)(R₁₄), or R₃ₐ and R_{3b} together form ═O, or R₃ₐ and R₄ₐ together form a bond;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CONH₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of −OR₁₃, —N(R₁₄)(R₁₄), CO₂R₁₃, and —C(O)N(R₁₄)(R₁₄); or R₄ₐ and R_{4b} together form ═O, ═CH₂, ═CHF, or ═CF₂, or R₄ₐ and R₁ₐ together form a bond;
R_{4b} is selected from the group consisting of hydrogen, halogen, C₁-C₄ aliphatic, and C₁-C₄ haloaliphatic, or R₄ₐ and R_{4b} together form ═O, ═CH₂, ═CHF, or ═CF₂;
R₅ is a ring selected from the group consisting of: 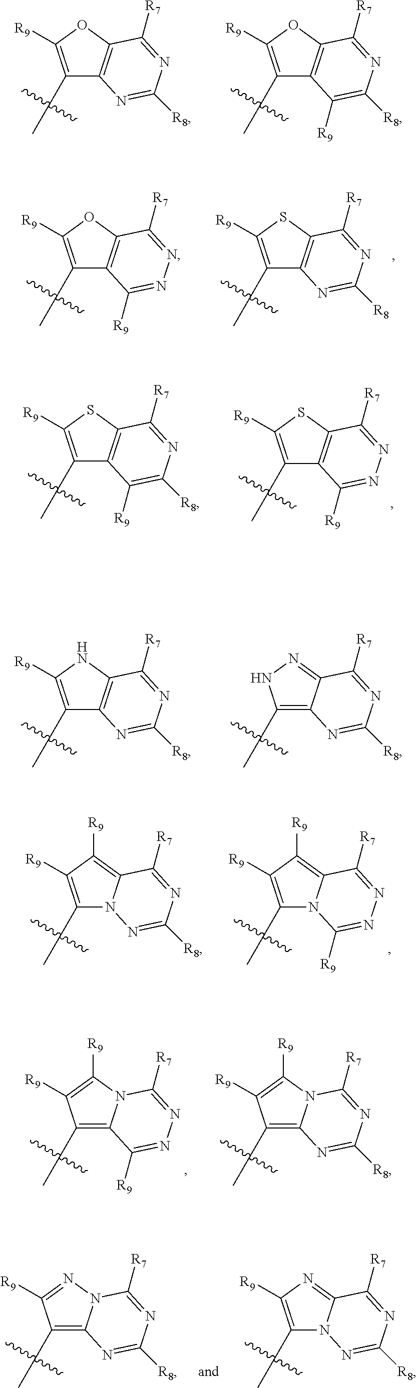
wherein one ring nitrogen atom is optionally oxidized;
R₆ is hydrogen, or C₁-C₄ aliphatic;
wherein R₇ is independently selected from hydrogen, halogen, —CN, —OH, —OR₁₂, —SH, —SR₁₂, —NH₂, —NHR₁₂, —N(R₁₂)₂, and —R₁₅;
each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
each R₁₀ is are independently hydrogen, halogen, —OH, optionally substituted C₁-C₄ aliphatic, or C₁-C₄ haloaliphatic;
each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
R₁₆ₐ is optionally substituted aryl, optionally substituted heteroaryl, optionally substituted heterocycyl, or optionally substituted cycloaliphatic group;
R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
R₁₇ is independently hydrogen, optionally substituted C₁-C₆ aliphatic, optionally substituted aryl, optionally substituted heteroaryl, or optionally substituted heterocyclyl;
R₁₈ is independently hydrogen, optionally substituted C₁-C₆ aliphatic, optionally substituted aryl, optionally substituted heteroaryl, or optionally substituted heterocyclyl;
R₁₉ is independently optionally substituted C₁-C₆ aliphatic, optionally substituted aryl, or optionally substituted heteroaryl; and
R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

2. A compound according to claim 1, where:
m is 1, or 2;
X is —CH₂—, —CH(halogen)-, —O—, —NH—, or —N(C₁-C₆ alkyl)-;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₃ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CONH₂, —CON(H)R₁₂, —OC(O)N(H)R₁₂, —C(O)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄), or R₃ₐ and R_{3b} together form ═O, or R₃ₐ and R₄ₐ together form a bond;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄); or R₄ₐ and R_{4b} together form ═O, ═CH₂, ═CHF, or ═CF₂, or R₄ₐ and R₃ₐ together form a bond;
R_{4b} is selected from the group consisting of hydrogen, halogen, C₁-C₄ aliphatic, and C₁-C₄ haloaliphatic, or R₄ₐ and R_{4b} together form ═O, ═CH₂, ═CHF, or ═CF₂;
R₅ is a ring selected from the group consisting of: 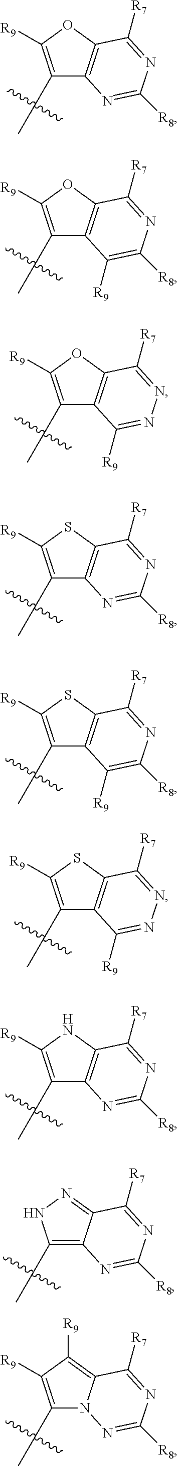 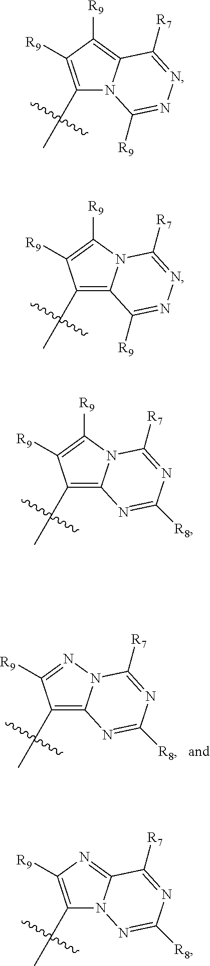
wherein one ring nitrogen atom is optionally oxidized;
R₆ is hydrogen, or C₁-C₄ aliphatic;
wherein R₇ is independently selected from hydrogen, halogen, —CN, —OH, —OR₁₂, —SH, —SR₁₂, —NH₂, —NHR₁₂, —N(R₁₂)₂, and —R₁₅;
each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
each R₁₀ is are independently hydrogen, halogen, —OH, optionally substituted C₁-C₄ aliphatic, or C₁-C₄ haloaliphatic;
each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
R₁₆ₐ is optionally substituted aryl, optionally substituted heteroaryl, optionally substituted heterocycyl, or optionally substituted cycloaliphatic group;
R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
R₁₇ is independently hydrogen, optionally substituted C₁-C₆ aliphatic, optionally substituted aryl, optionally substituted heteroaryl, or optionally substituted heterocyclyl;
R₁₈ is independently hydrogen, optionally substituted C₁-C₆ aliphatic, optionally substituted aryl, optionally substituted heteroaryl, or optionally substituted heterocyclyl;
R₁₉ is independently optionally substituted C₁-C₆ aliphatic, optionally substituted aryl, or optionally substituted heteroaryl; and
R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

3. A compound according to claim 1, where:
m is 1, or 2;
X is —CH₂—, —CH(halogen)-, —O—, —NH—, or —N(C₁-C₆ alkyl)-;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₃ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄), or R₃ₐ and R_{3b} together form ═O;
R_{3b} is selected from the group consisting of hydrogen, or R₃ₐ and R_{3b} together form ═O;
R₄ₐ is selected from the group consisting of hydrogen, halogen, —CN, —N₃, —OH, —OR₁₂, —NH₂, —NH(R₁₂), —N(H)CO₂R₁₂, —N(H)C(O)R₁₂, —CON(H)R₁₂, —OC(O)N(H)R₁₂, —OC(O)R₁₂, —OC(O)OR₁₂, —OS(O)₂NH₂, C₁-C₄ haloaliphatic, and C₁-C₄ aliphatic optionally substituted with one or two substituents independently selected from the group consisting of —OR₁₃, —N(R₁₄)(R₁₄), —CO₂R₁₃, and —C(O)N(R₁₄)(R₁₄);
R_{ob} is selected from the group consisting of hydrogen, halogen, C₁-C₄ aliphatic, and C₁-C₄ haloaliphatic;
R₅ is a ring selected from the group consisting of: 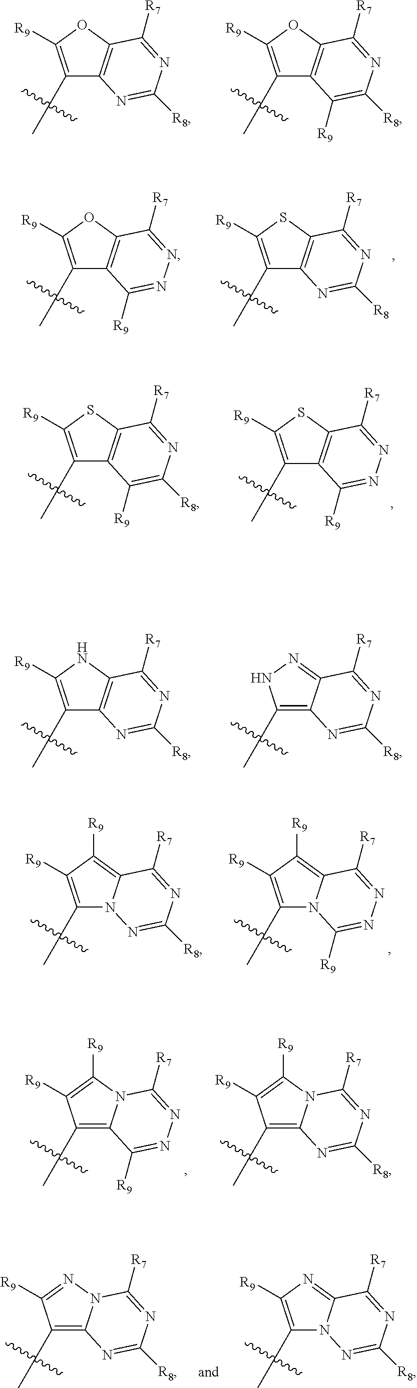
wherein one ring nitrogen atom is optionally oxidized;
R₆ is hydrogen, or C₁-C₄ aliphatic;
wherein R₇ is independently selected from hydrogen, halogen, —CN, —OH, —OR₁₂, —SH, —SR₁₂, —NH₂, —NHR₁₂, —N(R₁₂)₂, and —R₁₅;
each R₈ is independently hydrogen, halogen, —CN, —OH, —OR₁₀, —SH, —SR₁₀, —NH₂, —NHR₁₀, —N(R₁₀)₂, or optionally substituted C₁-C₄ aliphatic group;
each R₉ is independently selected from hydrogen, halogen, —CN, —OR₁₀, —SR₁₀, —N(R₁₀)(R₁₁), optionally substituted C₁-C₆ aliphatic, optionally substituted aryl, optionally substituted heteroaryl, and optionally substituted heterocyclyl;
each R₁₀ is are independently hydrogen, halogen, —OH, optionally substituted C₁-C₄ aliphatic, or C₁-C₄ haloaliphatic;
each R₁₁ is independently hydrogen, halogen, —OH, optionally substituted C₁-C₄ aliphatic, or C₁-C₄ haloaliphatic; or R₁₀ and R₁₁ together form ═O, ═CH₂, ═CHF, ═CF₂, ═CH(C₁-C₆ alkyl), or ═C(C₁-C₆ alkyl)₂;
each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
each R₁₃ is independently selected from hydrogen, optionally substituted C₁-C₄ aliphatic, C₁-C₄ haloaliphatic, optionally substituted aryl, and optionally substituted aryl(C₁-C₄ alkyl);
each R₁₄ is independently selected from hydrogen, C₁-C₆ aliphatic, C₁-C₆ haloaliphatic, optionally substituted aryl, optionally substituted aryl(C₁-C₆ alkyl), optionally substituted heteroaryl, and optionally substituted heterocyclyl, or two R¹⁴ taken together with the nitrogen atom to which they are attached form an optionally substituted heterocyclyl ring having, in addition to the nitrogen atom, 0-2 ring heteroatoms independently selected from N, O, and S;
R₁₅ is optionally substituted C₁-C₁₀ aliphatic, optionally substituted aryl, optionally substituted heteroaryl, optionally substituted heterocyclyl, -V-Z-R₁₆ₐ, -V-Z-R_{16b}, or —R_{16c};
wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—, —N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
R₁₆ₐ is optionally substituted aryl, optionally substituted heteroaryl, optionally substituted heterocycyl, or optionally substituted cycloaliphatic group;
R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—C(O)OR₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
R₁₇ is independently hydrogen, optionally substituted C₁-C₆ aliphatic, optionally substituted aryl, optionally substituted heteroaryl, or optionally substituted heterocyclyl;
R₁₈ is independently hydrogen, optionally substituted C₁-C₆ aliphatic, optionally substituted aryl, optionally substituted heteroaryl, or optionally substituted heterocyclyl;
R₁₉ is independently optionally substituted C₁-C₆ aliphatic, optionally substituted aryl, or optionally substituted heteroaryl; and
R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

4. A compound according to claim 1, where:
m is 1, or 2;
X is —CH₂—, —O—, or —NH—;
Y is —O—, or —CR₁₀R₁₁—;
R₁ₐ and R_{1b} are each independently hydrogen, halogen, C₁-C₄ aliphatic, or C₁-C₄ haloaliphatic;
R₂ is hydrogen, or C₁-C₄ aliphatic;
R₃ₐ is selected from the group consisting of hydrogen, halogen, —CN, —OH, —OR₁₂, —NH₂, —NH(R₁₂), —N(H)CO₂R₁₂, —N(H)C(O)R₁₂, —OC(O)N(H)R₁₂, —OC(O)R₁₂, —OC(O)OR₁₂, —OS(O)₂NH₂, optionally substituted C₁-C₄ haloaliphatic, and C₁-C₄ aliphatic;
R_{3b} is selected from the group consisting of hydrogen, halogen, C₁-C₄ aliphatic, and C₁-C₄ haloaliphatic, or R₃ₐ and R_{3b} together form ═O;
R₄ₐ is selected from the group consisting of hydrogen, halogen, —CN, —OH, —OR₁₂, —NH₂, —NH(R₁₂), —N(H)CO₂R₁₂, —N(H)C(O)R₁₂, —OC(O)N(H)R₁₂, —OC(O)R₁₂, —OC(O)OR₁₂, optionally substituted C₁-C₄ haloaliphatic, and C₁-C₄ aliphatic;
R_{4b} is selected from the group consisting of hydrogen, halogen, C₁-C₄ aliphatic, and C₁-C₄ haloaliphatic, or R₄ₐ and R_{4b} together form ═O or ═CH₂;
R₅ is a ring selected from the group consisting of: 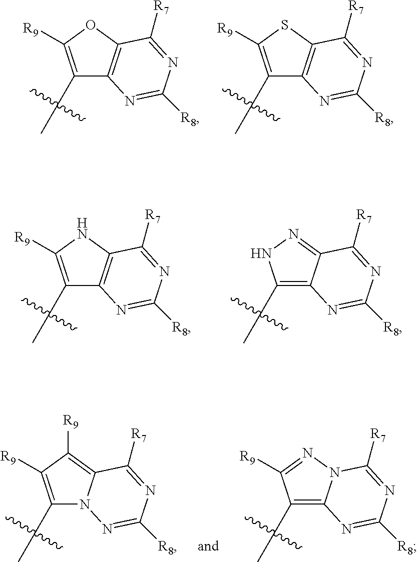
R₆ is hydrogen, or C₁-C₄ aliphatic;
wherein R₇ is independently selected from halogen, —OH, —OR₁₂, —SR₁₂, —NH₂, —NHR₁₂, —N(R₁₂)₂, and —R₁₅;
each R₈ is independently hydrogen, halogen, —CN, —OH, —OR₁₂, —SR₁₀, —NH₂, —NHR₁₀, —N(R₁₀)₂, or optionally substituted C₁-C₄ aliphatic group;
each R₉ is independently selected from hydrogen, halogen, —CN, —OR₁₀, —SR₁₀, —N(R₁₀)(R₁₁), optionally substituted C₁-C₆ aliphatic;
each R₁₀ is are independently hydrogen, halogen, —OH, optionally substituted C₁-C₄ aliphatic, or C₁-C₄ haloaliphatic;
each R₁₁ is independently hydrogen, halogen, —OH, optionally substituted C₁-C₄ aliphatic, or C₁-C₄ haloaliphatic;
each R₁₂ is independently an optionally substituted C₁-C₁₀ aliphatic, C₁-C₁₀ haloaliphatic, optionally substituted C₃-C₁₄ cycloaliphatic, optionally substituted aryl, optionally substituted heteroaryl, or optionally substituted heterocyclyl group;
R₁₅ is -V-Z-R₁₆ₐ, -V-Z-R_{16b}, —R_{16c}, or an optionally substituted aliphatic, aryl, heteroaryl, or heterocyclyl group;
wherein V is —N(R₁₇)—, —S(O)₂—, —S(O)—, —C(O)O—, —C(O)—, —C(NR₁₇)═N—, —C(═N(R₁₇))—N(R₁₇)—, —C(OR₁₇)═N—, —CON(R₁₇)—/—N(R₁₇)C(O)—, —N(R₁₇)C(O)N(R₁₇)—, —N(R₁₇)S(O)₂—, —N(R₁₇)SO₂—N(R₁₇)—, —N(R₁₇)CO₂—, —SO₂N(R₁₇)—, —OC(O)—, —OC(O)O—, —OC(O)N(R₁₇)—, or —N(R₁₇)—N(R₁₇)—;
Z is an optionally substituted C₁-C₆ alkylene chain, wherein the alkylene chain is optionally interrupted by —C(R₁₇)═C(R₁₇)—, —C≡C—, —O—, —S—, —N(R₁₇)—, —N(R₁₇)CO—, —N(R₁₇)CO₂—, —C(O)N(R₁₇)—, —C(O)—, —C(O)—C(O)—, —CO₂—, —OC(O)—, —OC(O)O—, —N(R₁₇)C(O)N(R₁₇)—, —N(R₁₇)N(R₁₇)—, —OC(O)N(R₁₇)—, —S(O)—, —S(O)₂—, —N(R₁₇)S(O)₂—, or —S(O)₂N(R₁₇);
R₁₆ₐ is optionally substituted aryl, optionally substituted heteroaryl, optionally substituted heterocycyl, or optionally substituted cycloaliphatic group;
R_{16b} is halogen, —NO₂, —CN, —OR₁₈, —SR₁₉, —N(R₂₀)₂, —N(R₂₀)C(O)R₁₉, —N(R₂₀)C(O)N(R₂₀)₂, —N(R₂₀)CO₂R₁₈, —O—CO₂—R₁₈, —OC(O)N(R₂₀)₂, —OC(O)R₁₈, —N(R₂₀)—N(R₂₀)₂, —N(R₂₀)—OR₁₉, —N(R₂₀)S(O)₂R₁₉, —N(R₂₀)SO₂—N(R₂₀)₂—C(R₁₈)═C(R₁₈)₂, —C≡C—R₁₈, —S(O)R₁₉, —SO₂R₁₉, —SO₂—N(R₂₀), —C(R₁₈)═N—OR₁₈, —CO₂R₁₈, —C(O)—C(O)R₁₈, —C(O)R₁₈, —C(O)N(R₂₀)₂, —C(═NR₂₀)—N(R₂₀)₂, or —C(═NR₂₀)—OR₁₈;
R_{16c} is —NO₂, —CN, —S(O)R₁₉, —SO₂R₁₉, —SO₂—N(R₂₀), —C(R₁₈)═N—OR₁₈, —N(R₂₀)C(O)R₁₉, —N(R₂₀)C(O)N(R₂₀)₂, —O—CO₂—R₁₈, —OC(O)N(R₂₀)₂, —OC(O)R₁₈, —CO₂R₁₈, —C(O)—C(O)R₁₈, —C(O)R₁₈, —C(O)N(R₂₀)₂, —C(═NR₂₀)—N(R₂₀)₂, —C(═NR₂₀)—OR₁₈, —N(R₂₀)—N(R₂₀)₂, —N(R₂₀)—OR₁₉, —N(R₂₀)S(O)₂R₁₉, or —N(R₂₀)SO₂—N(R₂₀)₂;
R₁₇ is independently hydrogen, optionally substituted C₁-C₆ aliphatic, optionally substituted aryl, optionally substituted heteroaryl, or optionally substituted heterocyclyl;
R₁₈ is independently hydrogen, optionally substituted C₁-C₆ aliphatic, optionally substituted aryl, optionally substituted heteroaryl, or optionally substituted heterocyclyl;
R₁₉ is independently optionally substituted C₁-C₆ aliphatic, optionally substituted aryl, or optionally substituted heteroaryl; and
R₂₀ is independently optionally substituted C₁-C₆ aliphatic, optionally substituted aryl, optionally substituted heteroaryl, or optionally substituted heterocyclyl, or two R₂₀ taken together with the nitrogen atom to which they are attached form an optionally substituted heterocyclyl ring having, in addition to the nitrogen atom, 0-2 ring heteroatoms independently selected from N, O, and S.

5. A compound according to claim 1, wherein m is 1.

6. A compound according to claim 5, wherein X is —O—, —CH₂—, or —NH—.

7. A compound according to claim 6, wherein X is —O—.

8. A compound according to claim 1, wherein Y is —O—, or —CR₁₀R₁₁—.

9. A compound according to claim 8, wherein Y is —O—.

10. A compound according to claim 8, wherein Y is —CR₁₀R₁₁—.

11. A compound according to claim 10, wherein R₁₀ and R₁₁ are independently hydrogen, halogen, —OH, or optionally substituted C₁-C₄ aliphatic.

12. A compound according to claim 11, wherein R₁₀ and R₁₁ are independently hydrogen.

13. A compound according to claim 1, wherein R₂ and R₆ are independently hydrogen.

14. A compound according to claim 1, wherein R₃ₐ is selected from the group consisting of hydrogen, —OH, and —OS(O)₂NH₂, or R₃ₐ and R_{ab} together form ═O.

15. A compound according to claim 14, wherein R₃ₐ is —OH.

16. A compound according to claim 1, wherein R_{3b} is hydrogen.

17. A compound according to claim 1, wherein R₃ₐ and R_{3b} together form ═O.

18. A compound according to claim 1, wherein R₄ₐ is selected from the group consisting of hydrogen, halogen, —OH, —OR₁₂, and optionally substituted C₁-C₄ aliphatic.

19. A compound according to claim 18, wherein R₄ₐ is —OH.

20. A compound according to claim 18, wherein R₄ₐ is hydrogen.

21. A compound according to claim 1, wherein R_{4b} is hydrogen.

22. A compound according to claim 1, wherein R₃ₐ is —OH, and R₄ₐ is hydrogen.

23. A compound according to claim 1, wherein R₃ₐ is —OH, and R₄ₐ is —OH.

24. A compound according to claim 22, wherein R_{3b} is hydrogen, and R_{4b} is hydrogen.

25. A compound according to claim 1, wherein R₅ is selected from the group consisting of:

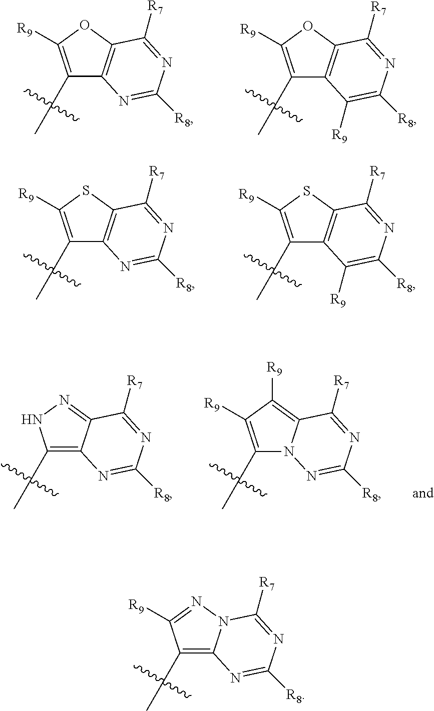

26. A compound according to claim 25, wherein R₅ is selected from the group consisting of:

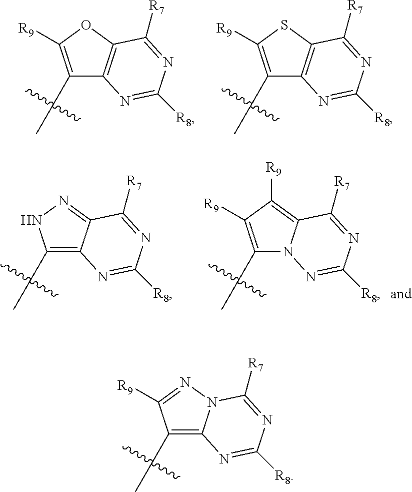

27. A compound according to claim 1, wherein R₇ is —NH₂, —NHR₁₂, or —R₁₅; each R₁₂ is independently an optionally substituted C₁-C₁₀ aliphatic, optionally substituted aryl, optionally substituted C₃-C₁₄ cycloaliphatic, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

28. A compound according to claim 27, wherein R₇ is —NH₂, or —NHR₁₂; and each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group.

29. A compound according to claim 28, wherein R₁₂ is optionally substituted aryl or optionally substituted C₃-C₁₄ cycloaliphatic.

30. A compound according to claim 29, wherein R₁₂ is optionally substituted 2,3-dihydro-1H-inden-1-yl.

31. A compound according to claim 27, wherein R₇ is —R₁₅; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

32. A compound according to claim 31, wherein R₇ is —R₁₅; and
R₁₅ is -V-Z-R_{16d};
where V is —NH—,
Z is an optionally substituted C₁-C₆ alkylene chain, and
R_{16d} is optionally substituted aryl, optionally substituted heteroaryl, optionally substituted heterocyclyl, or optionally substituted cycloaliphatic group.

33. A compound according to claim 32, wherein R₇ is —NH—CH₂—R_{16d}, where R_{16d} is phenyl optionally substituted with one or more of halogen, —NO₂, —CN, —OH, —NH₂, —NH(C₁-C₆ alkyl), —N(C₁-C₆ alkyl)₂, C₁-C₆ alkyl, and C₁-C₆ haloalkyl.

34. A compound according to claim 1, wherein R₅ is: 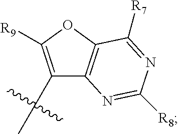
and
R₇ is —NH₂, —NHR₁₂, or —R₁₅;
each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

35. A compound according to claim 1, wherein R₅ is: 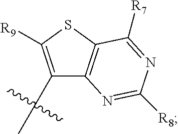
and
R₇ is —NH₂, —NHR₁₂, or —R₁₅;
each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

36. A compound according to claim 1, wherein R₅ is: 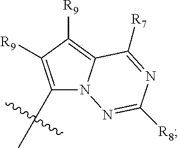
and
R₇ is —NH₂, —NHR₁₂, or —R₁₅;
each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

37. A compound according to claim 1, wherein R₅ is: 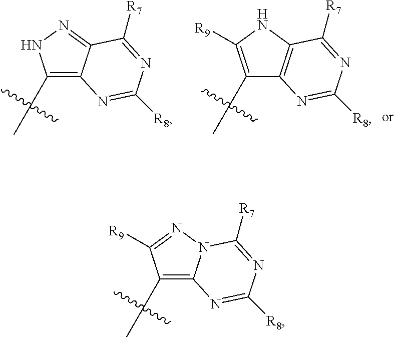
and
R₇ is —NH₂, —NHR₁₂, or —R₁₅;
each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

38. A compound according to claim 1, wherein R₅ is: 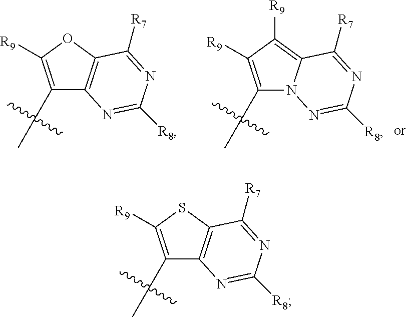
R₇ is —NH₂, —NHR₁₂, or —R₁₅;
each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

39. A compound according to claim 34, wherein R₇ is —NH₂, —NHR₁₂, or —R₁₅; each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

40. A compound according to claim 39, wherein R₇ is —NH₂, or —NHR₁₂; and each R₁₂ is independently an optionally substituted C₁-C₁₀ aliphatic, optionally substituted C₃-C₁₄ cycloaliphatic, optionally substituted aryl, optionally substituted heteroaryl, or optionally substituted heterocyclyl group.

41. A compound according to claim 40, wherein R₁₂ is optionally substituted 2,3-dihydro-1H-inden-1-yl.

42. A compound according to claim 39, wherein R₇ is —R₁₅; and
R₁₅ is -V-Z-R_{16d};
where V is —N(R₁₇)—,
Z is an optionally substituted C₁-C₆ alkylene chain, and
R_{16d} is halogen, —NO₂, —CN, —OR₁₈, —SR₁₉, —N(R₂₀)₂, optionally substituted aryl, optionally substituted heteroaryl, optionally substituted heterocyclyl, or optionally substituted cycloaliphatic group.

43. A compound according to claim 42, wherein R₇ is —R₁₅; and
R₁₅ is -V-Z-R_{16d};
where V is —NH—,
Z is an optionally substituted C₁-C₆ alkylene chain, and
R_{16d} is optionally substituted aryl, optionally substituted heteroaryl, optionally substituted heterocycyl, or optionally substituted cycloaliphatic group.

44. A compound according to claim 43, wherein R₇ is —NH—CH₂—R_{16d}, where R_{16d} is phenyl optionally substituted with one or more of halogen, —NO₂, —CN, —OH, —NH₂, —NH(C₁-C₆ alkyl), —N(C₁-C₆ alkyl)₂, C₁-C₆ alkyl, and C₁-C₆ haloalkyl.

45. A compound according to claim 1, wherein R₂, R₃ₐ, R_{3b}, R₄ₐ, R₄ₐ, and R₅ have the following configuration when Y is —O—:

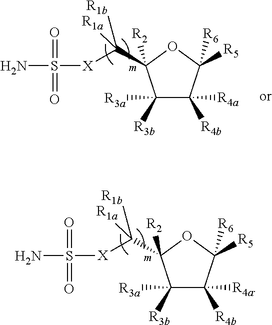

46. A compound according to claim 45, wherein the configuration is:

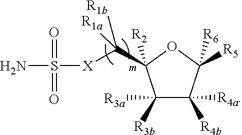

47. A compound according to claim 1, wherein R₂, R₃ₐ, R_{3b}, R₄ₐ, R₄ₐ, and R₅ have the following configuration when Y is —CH₂—:

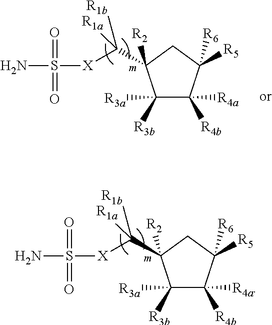

48. A compound according to claim 47, wherein the configuration is:

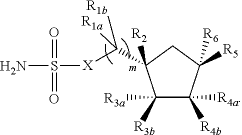

49. A compound according to claim 1, which is:
[(2R,5R)-5-[4-[[(1S)-indan-1-yl]amino]pyrrolo[2,1-f][1,2,4]triazin-7-yl]-3-oxo-tetrahydrofuran-2-yl]methyl sulfamate;
[(2R,3S,5R)-3-hydroxy-5-[4-[[(1S)-indan-1-yl]amino]pyrrolo[2,1-f][1,2,4]triazin-7-yl]tetrahydrofuran-2-yl]methyl sulfamate;
[(2R,3S,4R,5S)-5-(7-amino-1H-pyrazolo[4,3-d]pyrimidin-3-yl)-3,4-dihydroxy-tetrahydrofuran-2-yl]methyl sulfamate;
[(2R,3S,4R,5S)-5-(4-aminothieno[3,2-d]pyrimidin-7-yl)-3,4-dihydroxy-tetrahydrofuran-2-yl]methyl sulfamate;
((2R,3S,4R,5S)-5-(4-((S)-2,3-dihydro-1H-inden-1-ylamino)thieno[3,2-d]pyrimidin-7-yl)-3,4-dihydroxytetrahydrofuran-2-yl)methyl sulfamate;
((2R,3S,4R,5S)-5-(4-(4-chlorobenzylamino)thieno[3,2-d]pyrimidin-7-yl)-3,4-dihydroxytetrahydrofuran-2-yl)methyl sulfamate;
[(1R,2S,4R)-2-hydroxy-4-[4-[[(1S)-indan-1-yl]amino]pyrrolo[2,1-f][1,2,4]triazin-7-yl]cyclopentyl]methyl sulfamate;
[(1S,2S,4R)-2-hydroxy-4-[4-[[(1S)-indan-1-yl]amino]pyrrolo[2,1-f][1,2,4]triazin-7-yl]cyclopentyl]methyl sulfamate;
[(1S,2S,4R)-2-hydroxy-4-[4-[[(1S)-indan-1-yl]amino]thieno[3,2-d]pyrimidin-7-yl]cyclopentyl]methyl sulfamate;
((2R,3S,5R)-5-(4-((S)-2,3-dihydro-1H-inden-1-ylamino)thieno[3,2-d]pyrimidin-7-yl)-3-hydroxytetrahydrofuran-2-yl)methyl sulfamate;
((2R,3S,4R,5S)-5-(4-aminofuro[3,2-d]pyrimidin-7-yl)-3,4-dihydroxytetrahydrofuran-2-yl)methyl sulfamate;
((2R,3S,4R,5S)-5-(4-((S)-2,3-dihydro-1H-inden-1-ylamino)furo[3,2-d]pyrimidin-7-yl)-3,4-dihydroxytetrahydrofuran-2-yl)methyl sulfamate;
((2R,3S,4R,5S)-5-(4-aminopyrazolo[1,5-a][1,3,5]triazin-8-yl)-3,4-dihydroxytetrahydrofuran-2-yl)methyl sulfamate;
((2R,3S,4R,5S)-5-(4-((S)-2,3-dihydro-1H-inden-1-ylamino)pyrazolo[1,5-a][1,3,5]triazin-8-yl)-3,4-dihydroxytetrahydrofuran-2-yl)methyl sulfamate;
((1R,2S,4R)-4-(4-((S)-2,3-dihydro-1H-inden-1-ylamino)pyrazolo[1,5-a][1,3,5]triazin-8-yl)-2-hydroxycyclopentyl)methyl sulfamate;
((2R,3S,4R,5S)-5-(4-((S)-2,3-dihydro-1H-inden-1-ylamino)-5H-pyrrolo[3,2-d]pyrimidin-7-yl)-3,4-dihydroxytetrahydrofuran-2-yl)methyl sulfamate;
[(1R,2S,4S)-2-[(aminosulfonyl)oxy]-4-{4-[(1S)-2,3-dihydro-1H-inden-1-ylamino]pyrrolo[2,1-f][1,2,4]triazin-7-yl}cyclopentyl]methyl sulfamate;
((1R,2S,4R)-4-(4-((S)-2,3-dihydro-1H-inden-1-ylamino)pyrrolo[1,2-f][1,2,4]triazin-7-yl)-2-hydroxycyclopentyl)methyl sulfamate;
[(1R,2S,4S)-2-[(aminosulfonyl)oxy]-4-{4-[(1S)-2,3-dihydro-1H-inden-1-ylamino]pyrazolo[1,5-a][1,3,5]triazin-8-yl}cyclopentyl]methyl sulfamate;
((1S,2S,4R)-4-(4-(2,3-dihydro-1H-inden-1-ylamino)thieno[3,2-d]pyrimidin-7-yl)-2-hydroxycyclopentyl)methyl sulfamate;
((1R,2S,4R)-4-(4-(2,3-dihydro-1H-inden-1-ylamino)thieno[3,2-d]pyrimidin-7-yl)-2-hydroxycyclopentyl)methyl sulfamate;
((2R,3S,4R,5R)-5-(4-((S)-2,3-dihydro-1H-inden-1-ylamino)thieno[3,2-d]pyrimidin-7-yl)-3,4-dihydroxytetrahydrofuran-2-yl)methyl sulfamate;
((2R,3S,4R,5S)-5-(4-(benzylamino)thieno[3,2-d]pyrimidin-7-yl)-3,4-dihydroxytetrahydrofuran-2-yl)methyl sulfamate;
((2R,3S,4R,5S)-5-(4-(cyclopropylmethylamino)thieno[3,2-d]pyrimidin-7-yl)-3,4-dihydroxytetrahydrofuran-2-yl)methyl sulfamate;
((2R,3S,4R,5S)-5-(4-(cyclopropylamino)thieno[3,2-d]pyrimidin-7-yl)-3,4-dihydroxytetrahydrofuran-2-yl)methyl sulfamate;
((2R,3S,4R,5S)-3,4-dihydroxy-5-(4-(2-methoxyethylamino)thieno[3,2-d]pyrimidin-7-yl)tetrahydrofuran-2-yl)methyl sulfamate;
((2R,3S,4R,5S)-5-(4-((S)-2,3-dihydro-1H-inden-1-ylamino)thieno[3,2-d]pyrimidin-7-yl)-3,4-dihydroxytetrahydrofuran-2-yl)methyl sulfamate;
((2R,3S,4R,5S)-5-(4-(2-chlorobenzylamino)thieno[3,2-d]pyrimidin-7-yl)-3,4-dihydroxytetrahydrofuran-2-yl)methyl sulfamate;
((2R,3S,4R,5S)-5-(4-(4-chlorobenzylamino)pyrazolo[1,5-a][1,3,5]triazin-8-yl)-3,4-dihydroxytetrahydrofuran-2-yl)methyl sulfamate;
((2R,3S,4R,5R)-5-(4-((S)-2,3-dihydro-1H-inden-1-ylamino)pyrazolo[1,5-a][1,3,5]triazin-8-yl)-3,4-dihydroxytetrahydrofuran-2-yl)methyl sulfamate;
((2R,3S,4R,5S)-5-(4-((R)-2,3-dihydro-1H-inden-1-ylamino)furo[3,2-d]pyrimidin-7-yl)-3,4-dihydroxytetrahydrofuran-2-yl)methyl sulfamate;
((2R,3S,4R,5S)-5-(4-(2,4-dichlorobenzylamino)furo[3,2-d]pyrimidin-7-yl)-3,4-dihydroxytetrahydrofuran-2-yl)methyl sulfamate;
((2R,3S,4R,5S)-5-(4-(4-chloro-3-(trifluoromethyl)benzylamino)furo[3,2-d]pyrimidin-7-yl)-3,4-dihydroxytetrahydrofuran-2-yl)methyl sulfamate; or
((2R,3S,4R,5S)-3,4-dihydroxy-5-(4-((1R,2R)-2-hydroxy-2,3-dihydro-1H-inden-1-ylamino)furo[3,2-d]pyrimidin-7-yl)tetrahydrofuran-2-yl)methyl sulfamate;
((1S,2S,4R)-4-(4-((S)-2,3-dihydro-1H-inden-1-ylamino)furo[3,2-d]pyrimidin-7-yl)-2-hydroxycyclopentyl)methyl sulfamate;
((2R,3S,4R,5S)-5-(4-(3-chlorobenzylamino)thieno[3,2-d]pyrimidin-7-yl)-3,4-dihydroxytetrahydrofuran-2-yl)methyl sulfamate;
((1S,2S,4S)-4-(4-((S)-2,3-dihydro-1H-inden-1-ylamino)furo[3,2-d]pyrimidin-7-yl)-2-hydroxycyclopentyl)methyl sulfamate;
((1S,2S,4R)-4-(4-(5-fluoro-2,3-dihydro-1H-inden-1-ylamino)thieno[3,2-d]pyrimidin-7-yl)-2-hydroxycyclopentyl)methyl sulfamate;
((1S,2S,4R)-2-hydroxy-4-(4-((5-methyl-2,3-dihydro-1H-inden-1-yl)amino)thieno[3,2-d]pyrimidin-7-yl)cyclopentyl)methyl sulfamate;
((1S,2S,4R)-4-(4-(((R)-4-bromo-2,3-dihydro-1H-inden-1-yl)amino)thieno[3,2-d]pyrimidin-7-yl)-2-hydroxycyclopentyl)methyl sulfamate;
((1S,2S,4R)-2-hydroxy-4-(4-(((S)-1,2,3,4-tetrahydronaphthalen-1-yl)amino)thieno[3,2-d]pyrimidin-7-yl)cyclopentyl)methyl sulfamate;
(((1S,2S,4R)-2-hydroxy-4-(4-(((S)-1,2,3,4-tetrahydronaphthalen-1-yl)amino)thieno[3,2-d]pyrimidin-7-yl)cyclopentyl)methyl sulfamate;
((1S,2S,4R)-2-hydroxy-4-(4-((4-methyl-2,3-dihydro-1H-inden-1-yl)amino)thieno[3,2-d]pyrimidin-7-yl)cyclopentyl)methyl sulfamate;
((1S,2S,4R)-2-hydroxy-4-(4-(((S)-6-methoxy-2,3-dihydro-1H-inden-1-yl)amino)thieno[3,2-d]pyrimidin-7-yl)cyclopentyl)methyl sulfamate;
((1S,2S,4R)-4-(4-(((S)-6,7-dihydro-5H-indeno[5,6-d][1,3]dioxol-5-yl)amino)thieno[3,2-d]pyrimidin-7-yl)-2-hydroxycyclopentyl)methyl sulfamate;
((1S,2S,4R)-2-hydroxy-4-(4-(((S)-4-methoxy-2,3-dihydro-1H-inden-1-yl)amino)thieno[3,2-d]pyrimidin-7-yl)cyclopentyl)methyl sulfamate;
((1S,2S,4R)-4-(4-(((R)-2,3-dihydro-1H-inden-1-yl)amino)thieno[3,2-d]pyrimidin-7-yl)-2-hydroxycyclopentyl)methyl sulfamate;
((1S,2S,4R)-4-(4-((4-chlorobenzyl)amino)thieno[3,2-d]pyrimidin-7-yl)-2-hydroxycyclopentyl)methyl sulfamate;
((1S,2S,4R)-4-(4-(((R)-2,3-dihydro-1H-inden-1-yl)(sulfamoyl)amino)thieno[3,2-d]pyrimidin-7-yl)-2-hydroxycyclopentyl)methyl sulfamate;
((1S,2S,4R)-4-(4-((cyclohexylmethyl)amino)thieno[3,2-d]pyrimidin-7-yl)-2-hydroxycyclopentyl)methyl sulfamate;
((1S,2S,4R)-4-(4-((4-bromo-2,3-dihydro-1H-inden-1-yl)amino)thieno[3,2-d]pyrimidin-7-yl)-2-hydroxycyclopentyl)methyl sulfamate;
((1S,2S,4R)-2-hydroxy-4-(4-((4-phenyl-2,3-dihydro-1H-inden-1-yl)amino)thieno[3,2-d]pyrimidin-7-yl)cyclopentyl)methyl sulfamate;
((1S,2S,4R)-4-(4-(((S)-1-cyclohexylethyl)amino)thieno[3,2-d]pyrimidin-7-yl)-2-hydroxycyclopentyl)methyl sulfamate;
((1S,2S,4R)-2-hydroxy-4-(4-(((5-methylfuran-2-yl)methyl)amino)thieno[3,2-d]pyrimidin-7-yl)cyclopentyl)methyl sulfamate;
((1S,2S,4R)-4-(4-((cyclopentylmethyl)amino)thieno[3,2-d]pyrimidin-7-yl)-2-hydroxycyclopentyl)methyl sulfamate;
((1S,2S,4R)-4-(4-(hexylamino)thieno[3,2-d]pyrimidin-7-yl)-2-hydroxycyclopentyl)methyl sulfamate;
((1S,2S,4R)-4-(4-aminothieno[3,2-d]pyrimidin-7-yl)-2-hydroxycyclopentyl)methyl sulfamate;
or a pharmaceutically acceptable salt of any of the above compounds.

50. A pharmaceutical composition comprising a compound according to claim 1 and a pharmaceutically acceptable carrier, solvent, adjuvant or diluent.

51. A method of decreasing an E1 enzyme activity in a sample, comprising contacting a sample with one or more compounds according to claim 1.

52. A method of claim 51, wherein the E1 enzyme is selected from the group consisting of NAE, UAE, and SAE.

53. A method of claim 52, wherein the E1 enzyme is NAE.

54. A method of treating a subject suffering from a disease or disorder selected from cancer, an inflammatory disorder, a neurodegenerative disorder, inflammation associated with infection, and cachexia disorder, the method comprising administering to a subject in need of such treatment an effective amount of one or more compounds according to claim 1.

55. A method of claim 54, wherein the disorder is cancer.

56. A method of claim 55, wherein the cancer is lung cancer, colorectal cancer, ovarian cancer, or a hematological cancer.

57. A method of claim 54, wherein the disorder is an immune response or vascular cell proliferation disorder.
